SEQUENCE LISTING (91 sequences)

SEQ ID NO: 1 - protein, 5 aa, Artificial Sequence
  RGS-peptide derivative
GGRGS

SEQ ID NO: 2 - protein, 5 aa, Artificial Sequence
  RGS-peptide derivative
DDRGS

SEQ ID NO: 3 - protein, 8 aa, Artificial Sequence
  reporter peptide (8-amino acid ligand RGS- peptide)
RGSIDTWV

SEQ ID NO: 4 - protein, 10 aa, Artificial Sequence
  N-terminally extended reporter peptide
GGRGSIDTWV

SEQ ID NO: 5 - protein, 10 aa, Artificial Sequence
  N-terminally extended reporter peptide
DDRGSIDTWV

SEQ ID NO: 6 - protein, 10 aa, Artificial Sequence
  fluorescently quenched TVMV substrate peptide
  MOD_RES (1)..(1): Xaa is 5-Amino-2-nitrobenzoic acid
  MOD_RES (10)..(10): Xaa is 7-Methoxycoumarin-4-yl
XETVRFQSKX

SEQ ID NO: 7 - DNA, 67 bp, Artificial Sequence
  “kissing loop” coding sequence with linker
ggtagtgaggtagttagcagatacctcactaccaacacacacacaacacacacacaacacacaagct

SEQ ID NO: 8 - DNA, 27 bp, Artificial Sequence
  OligoDNA1-Asp-amine/thiol/biotin
gacaggcaggtattctaaccgactgaa

SEQ ID NO: 9 - DNA, 25 bp, Artificial Sequence
  OligoDNA1-Asn-amine
ttaacagtccgccgttctaccgact

SEQ ID NO: 10 - DNA, 26 bp, Artificial Sequence
  OligoDNA1-Glu-amine/biotin
gtgaaagggcggtgtcctgggcctct

SEQ ID NO: 11 - DNA, 26 bp, Artificial Sequence
  OligoDNA1-Ile-amine
ttatcaggggtgcgctctaaccacct

SEQ ID NO: 12 - DNA, 30 bp, Artificial Sequence
  OligoDNA2-Asn-amine
ctcctctgactggactcgaaccagtgacat

SEQ ID NO: 13 - DNA, 843 bp, Artificial Sequence
  Temple A (eGFP template with GenBank number of KJ541667.1, based on pLTE vector)
  misc_feature (61)..(63): n is a, c, g, or t
atgacagtaatgtataaagtctgtaaagacattaaacacgtaagtgaaaccatggagatcnnntcgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctatacaaggagcagaagctgatctcggaggaggatctgcaagcttgtcgacctctagaggatccccggggctaa

SEQ ID NO: 14 - DNA, 816 bp, Artificial Sequence
  Template B (sGFP template, based on pOPINE vector with GenBank number of EF372397.1).)
  misc_feature (37)..(39): n is a, c, g, or t
  misc_feature (487)..(489): n is a, c, g, or t
atgacagaacagaagttgatctcggaagaagatttgnnntcgaagggagaagaattgtttacaggagtggtgccaatcttggtggaattggacggagatgtgaacggacacaagttctcggtgcgtggagaaggagaaggagacgcgacaaacggaaagttgacattgaagttcatctgcacaacaggaaagttgccagtgccatggccaacattggtgacaacattgacatacggagtgcagtgcttttcgcgttacccagatcacatgaagcgtcatgacttcttcaagtcggcgatgccagaaggatacgtgcaggaacgtacaatctcgttcaaggacgacggaacatacaagacacgtgcggaagtgaagttcgaaggagacacattggtgaatcgtattgaattgaagggaatcgacttcaaggaggatggaaacattttgggacacaagttggagtacaacttcaactcgcacaacgtgnnnatcacagcggacaagcagaagaacggaatcaaggcgaacttcaagattcgtcacaacgtggaggacggatcggtgcagttggcggatcactaccagcagaatacaccaattggagatggaccagtgttgttgccagacaatcactacttgtcgacacagtcggtgttgtcgaaggacccaaacgaaaagcgtgaccacatggtgttgttggaatttgtgacagcggcgggaattacacatggaatggacgaattgtacaagggaggattggaggtgttgttccagggaccaggacgtggatcgatcgacacatgggtgtgatgatga

SEQ ID NO: 15 - DNA, 714 bp, Artificial Sequence
  sGFP_T2
atgtcgaagggagaagaattgttcacaggagtggtgccaatcttggtggaattggacggagatgtgaacggacacaagttctcggtgcggggagaaggagaaggagacgcgacaaacggaaagttgacattgaagttcatctgcacaacaggaaagttgccagtgccatggccaacattggtgacaacattgacatacggagtgcagtgcttttcgcggtacccagatcacatgaagcggcatgacttcttcaagtcggcgatgccagaaggatacgtgcaggaacggacaatctcgttcaaggacgacggaacatacaagacaagggcggaagtgaagttcgaaggagacacattggtgaatcggattgaattgaagggaatcgacttcaaggaggatggaaacattttgggacacaagttggagtacaacttcaacagccacaacgtgtacatcacagcggacaagcagaagaacggaatcaaggcgaacttcaagattcggcacaacgtggaggacggatcggtgcagttggcggatcactaccagcagaatacaccaattggagatggaccagtgttgttgccagacaatcactacttgtcgacacagtcggtgttgtcgaaggacccaaacgaaaagcgggaccacatggtgctattggaatttgtgacagcggcgggaattacacatggaatggacgaattgtacaag

SEQ ID NO: 16 - DNA, 546 bp, Artificial Sequence
  CaM
atggaacagaagctgatttccgaagaggatctgggataggatcagctgacggaagagcagatcgcggaattcaaggaagcgttctccctgttcgacaaggacggagacggaacgattacgacgaaggaactgggaacggtgatgcgttccctgggacagaatccgacggaagcggagctgcaggacatgatcaacgaagtggatgcggacggaaacggaacgattgacttcccggaattcctgacgatgatggcgcgtaagatgaaggatacggactccgaagaggaaatccgtgaagcgtttcgtgtgttcgacaaggatggaaatggatacatttccgcggcggagctgcgtcacgtgatgacgaacctgggagagaagctgacggacgaagaggtggacgaaatgattcgtgaagcggacattgacggagacggacaggtgaattacgaagagttcgtgcagatgatgacggcgaggggaggactggaggtgctgttccagggaccgggacgtggatccatcgacacgtgggtgtgatgatga

SEQ ID NO: 17 - DNA, 20 bp, Artificial Sequence
  T7
gtaatacgactcactatagg

SEQ ID NO: 18 - DNA, 60 bp, Artificial Sequence
  MjY1 F1
gtaatacgactcactatagggagactaaggcgggctgatgagtccgtgaggacgaaacgg

SEQ ID NO: 19 - DNA, 60 bp, Artificial Sequence
  MjY1 F2
ccgtgaggacgaaacggtacccggtaccgtccccgccttagttcagagggcagaacggcg

SEQ ID NO: 20 - DNA, 42 bp, Artificial Sequence
  MjY1 R1
gaacccgtgccatgcggatttagagtccgccgttctgccctc

SEQ ID NO: 21 - DNA, 38 bp, Artificial Sequence
  MjY1 R2
tggtcccgcctacgggatttgaacccgtgccatgcgga

SEQ ID NO: 22 - DNA, 20 bp, Artificial Sequence
  MjY1 R3
tggtcccgcctacgggattt

SEQ ID NO: 23 - DNA, 60 bp, Artificial Sequence
  MjY2 F1
gtaatacgactcactatagggagactacggccggctgatgagtccgtgaggacgaaacgg

SEQ ID NO: 24 - DNA, 60 bp, Artificial Sequence
  MjY2 F2
ccgtgaggacgaaacggtacccggtaccgtcccggccgtagttcagcagggcagaacggc

SEQ ID NO: 25 - DNA, 43 bp, Artificial Sequence
  MjY2 R1
gaacccatgccatgcggatttagagtccgccgttctgccctgc

SEQ ID NO: 26 - DNA, 38 bp, Artificial Sequence
  MjY2 R2
tggtccggccgatgggatttgaacccatgccatgcgga

SEQ ID NO: 27 - DNA, 20 bp, Artificial Sequence
  MjY2 R3
tggtccggccgatgggattt

SEQ ID NO: 28 - DNA, 60 bp, Artificial Sequence
  MjY3 F1
gtaatacgactcactatagggagactagggcgggctgatgagtccgtgaggacgaaacgg

SEQ ID NO: 29 - DNA, 60 bp, Artificial Sequence
  MjY3 F2
ccgtgaggacgaaacggtacccggtaccgtccccgccctagttcagaggcagaacggcgg

SEQ ID NO: 30 - DNA, 41 bp, Artificial Sequence
  MjY3 R1
gaacccgagccatgcggatttagagtccgccgttctgcctc

SEQ ID NO: 31 - DNA, 38 bp, Artificial Sequence
  MjY3 R2
tggtcccgccctcgggatttgaacccgagccatgcgga

SEQ ID NO: 32 - DNA, 20 bp, Artificial Sequence
  MjY3 R3
tggtcccgccctcgggattt

SEQ ID NO: 33 - DNA, 60 bp, Artificial Sequence
  MjY4 F1
gtaatacgactcactatagggagactaccgccggctgatgagtccgtgaggacgaaacgg

SEQ ID NO: 34 - DNA, 60 bp, Artificial Sequence
  MjY4 F2
ccgtgaggacgaaacggtacccggtaccgtcccggcggtagttcagcagggcagaacggc

SEQ ID NO: 35 - DNA, 43 bp, Artificial Sequence
  MjY4 R1
gaacccctgccatgcggatttagagtccgccgttctgccctgc

SEQ ID NO: 36 - DNA, 38 bp, Artificial Sequence
  MjY4 R2
tggtccggcggaggggatttgaacccctgccatgcgga

SEQ ID NO: 37 - DNA, 20 bp, Artificial Sequence
  MjY4 R3
tggtccggcggaggggattt

SEQ ID NO: 38 - DNA, 47 bp, Artificial Sequence
  PylT(u25c) F1
ctaatacgactcactataggaaacctgatcatgtagatcgaacggac

SEQ ID NO: 39 - DNA, 41 bp, Artificial Sequence
  PylT(u25c) R1
taacccggctgaacggatttagagtccgttcgatctacatg

SEQ ID NO: 40 - DNA, 36 bp, Artificial Sequence
  PylT(u25c) R2
tggcggaaaccccgggaatctaacccggctgaacgg

SEQ ID NO: 41 - DNA, 20 bp, Artificial Sequence
  PylT(u25c) R3
tggcggaaaccccgggaatc

SEQ ID NO: 42 - DNA, 50 bp, Artificial Sequence
  tRNATyr(cua) F1
gtaatacgactcactataggtggggttcccgagcggccaaagggagcaga

SEQ ID NO: 43 - DNA, 53 bp, Artificial Sequence
  tRNATyr(cua) R1
gaaccttcgaagtcgatgacggcagatttagagtctgctccctttggccgctc

SEQ ID NO: 44 - DNA, 46 bp, Artificial Sequence
  tRNATyr(cua) R2
tggtggtgggggaaggattcgaaccttcgaagtcgatgacggcaga

SEQ ID NO: 45 - DNA, 20 bp, Artificial Sequence
  tRNATyr(cua) R3
tggtggtgggggaaggattc

SEQ ID NO: 46 - DNA, 48 bp, Artificial Sequence
  RNACys(ccu) F1
gtaatacgactcactataggcgcgttaacaaagcggttatgtagcgga

SEQ ID NO: 47 - DNA, 42 bp, Artificial Sequence
  RNACys(ccu) R1_ccu
gaaccggactagacggattaggaatccgctacataaccgctt

SEQ ID NO: 48 - DNA, 37 bp, Artificial Sequence
  RNACys(ccu) R2
tggaggcgcgttccggagtcgaaccggactagacgga

SEQ ID NO: 49 - DNA, 20 bp, Artificial Sequence
  RNACys(ccu) R3
tggaggcgcgttccggagtc

SEQ ID NO: 50 - DNA, 42 bp, Artificial Sequence
  tRNACys(cua) R1_cua
gaaccggactagacggatttagaatccgctacataaccgctt

SEQ ID NO: 51 - DNA, 30 bp, Artificial Sequence
  FortRNAArg(CCU)depletion
tgtcccctgcaggaatcgaacctgcaatta

SEQ ID NO: 52 - DNA, 30 bp, Artificial Sequence
  FortRNAArg(UCU)depletion
agaaggtcgttgctctatccaactgagcta

SEQ ID NO: 53 - RNA, 27 nt, Artificial Sequence
  Figure 2 - RGS1
augcggggcuccaucgacaccuggguc

SEQ ID NO: 54 - RNA, 36 nt, Artificial Sequence
  Figure 2 - RGS2
  misc_feature (10)..(12): n is a, c, g, or u
auggaugaunnncggggcuccaucgacaccuggguc

SEQ ID NO: 55 - protein, 9 aa, Artificial Sequence
  Figure 2 - RGS1
MRGSIDTWV

SEQ ID NO: 56 - protein, 12 aa, Artificial Sequence
  Figure 2 - RGS2
  MOD_RES (4)..(4): Xaa is any natural or non-natural amino acid
MDDXRGSIDTWV

SEQ ID NO: 57 - RNA, 50 nt, Artificial Sequence
  Figure 8B tDNA starting from A/T
  misc_feature (7)..(10): n is a, c, g, or u
gggagannnncugaugaguccgugaggacgaaacgguacccgguaccguc

SEQ ID NO: 58 - protein, 11 aa, Artificial Sequence
  Figure 11 for 12 amino acids
PAMFYHQNKEC

SEQ ID NO: 59 - DNA, 87 bp, Artificial Sequence
  Figure 16A tRNASer(GCU) isoacceptor
  modified_base (17)..(17): gm
  modified_base (19)..(20): d
  modified_base (41)..(41): p
  modified_base (66)..(66): p
ggugagguguccgagugguugaaggagcacgccugcuaaguguguauacggcaacguaugggggtucgaaucccccccucaccgcca

SEQ ID NO: 60 - RNA, 21 nt, Artificial Sequence
  Figure 16 - kissing loop
gggguaguuagcagauaccuc

SEQ ID NO: 61 - RNA, 17 nt, Artificial Sequence
  Figure 16 - tRNA(GCU) anticodon loop
cacgccugcuaagugug

SEQ ID NO: 62 - RNA, 17 nt, Artificial Sequence
  Figure 16 - tRNA(CGA) anticodon loop
ccggucucgaaaaccgg

SEQ ID NO: 63 - RNA, 17 nt, Artificial Sequence
  Figure 16 - tRNA(UGA) anticodon loop
ccggucuugaaaaccgg

SEQ ID NO: 64 - RNA, 17 nt, Artificial Sequence
  Figure 16 - tRNA(GGA) anticodon loop
cuccccuggaaagggag

SEQ ID NO: 65 - protein, 251 aa, Artificial Sequence
  Figure 17 peptide sequence
MEISSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEQKLISEEDL

SEQ ID NO: 66 - RNA, 74 nt, Artificial Sequence
  Figure 19 - tRNA(Cys)CCU
ggcgcguuaacaaagcgguuauguagcggauuccuaauccgucuaguccgguucgacuccggaacgcgccucca

SEQ ID NO: 67 - RNA, 74 nt, Artificial Sequence
  Figure 19 - wt-tRNA(Cys)GCA
ggcgcguuaacaaagcgguuauguagcggauugcaaauccgucuaguccgguucgacuccggaacgcgccucca

SEQ ID NO: 68 - RNA, 74 nt, Artificial Sequence
  Figure 19 - tRNA(Cys)GCU
ggcgcguuaacaaagcgguuauguagcggauugcuaauccgucuaguccgguucgacuccggaacgcgccucca

SEQ ID NO: 69 - DNA, 76 bp, Artificial Sequence
  MjY1_tRNA
cccgccttagttcagagggcagaacggcggactctaaatccgcatggcacgggttcaaatcccgtaggcgggacca

SEQ ID NO: 70 - DNA, 77 bp, Artificial Sequence
  MjY2_tRNA
ccggccgtagttcagcagggcagaacggcggactctaaatccgcatggcatgggttcaaatcccatcggccggacca

SEQ ID NO: 71 - DNA, 75 bp, Artificial Sequence
  MjY3_Trna
cccgccctagttcagaggcagaacggcggactctaaatccgcatggctcgggttcaaatcccgagggcgggacca

SEQ ID NO: 72 - DNA, 77 bp, Artificial Sequence
  MjY4_tRNA
ccggcggtagttcagcagggcagaacggcggactctaaatccgcatggcaggggttcaaatcccctccgccggacca

SEQ ID NO: 73 - protein, 16 aa, Artificial Sequence
  Figure 31 - peptide 1
  MOD_RES (11)..(11): Xaa is AzF (p-azidophenylalanine)
LEYNYNSHNVXIMADK

SEQ ID NO: 74 - protein, 16 aa, Artificial Sequence
  Figure 31 - peptide 2
  MOD_RES (11)..(11): Xaa is AzF (p-azidophenylalanine)
  MOD_RES (13)..(13): Oxidised (Oxi) methionine
LEYNYNSHNVXIMADK

SEQ ID NO: 75 - protein, 16 aa, Artificial Sequence
  Figure 31 - peptide 3
LEYNYNSHNVYIMADK

SEQ ID NO: 76 - protein, 11 aa, Artificial Sequence
  Figure 31 - peptide 4
LEYNYNSHNVR

SEQ ID NO: 77 - protein, 12 aa, Artificial Sequence
  Figure 31 - peptide 5
  MOD_RES (9)..(9): Xaa is AzF (p-azidophenylalanine)
HVSETMEIXSSK

SEQ ID NO: 78 - protein, 9 aa, Artificial Sequence
  Figure 31 - peptide 6
HVSETMEIR

SEQ ID NO: 79 - protein, 177 aa, Artificial Sequence
  Figure 36 - peptide
  MOD_RES (11)..(11): Xaa is a non-natural amino acid
  MOD_RES (158)..(158): Xaa is a non-natural amino acid
EQKLISEEDLXDQLTEEQIAEFKEAFSLFDKDGDGTITTKELGTVMRSLGQNPTEAELQDMINEVDADGNGTIDFPEFLTMMARKMKDTDSEEEIREAFRVFDKDGNGYISAAELRHVMTNLGEKLTDEEVDEMIREADIDGDGQVNYEEFVQMMTAXGGLEVLFQGPGRGSIDTWV

SEQ ID NO: 80 - DNA, 27 bp, Artificial Sequence
  OligoDNA1-Asp-amine 3′-amine
  modified_base (27)..(27): 3′-amine
gacaggcaggtattctaaccgactgaa

SEQ ID NO: 81 - DNA, 25 bp, Artificial Sequence
  OligoDNA1-Asn-amine
  modified_base (25)..(25): 3′-amine
ttaacagtccgccgttctaccgact

SEQ ID NO: 82 - DNA, 26 bp, Artificial Sequence
  OligoDNA1-Glu-amine
  modified_base (26)..(26): 3′-amine
gtgaaagggcggtgtcctgggcctct

SEQ ID NO: 83 - DNA, 26 bp, Artificial Sequence
  OligoDNA1-Ile-amine
  modified_base (26)..(26): 3′-amine
ttatcaggggtgcgctctaaccacct

SEQ ID NO: 84 - DNA, 30 bp, Artificial Sequence; identical to SEQ ID NO: 12
  OligoDNA2-Asn-amine
  modified_base (30)..(30): 3′-amine

SEQ ID NO: 85 - DNA, 27 bp, Artificial Sequence
  OligoDNA1-Asp-biotin
  modified_base (27)..(27): 3′-biotin-triethylene glycol (TEG)
gacaggcaggtattctaaccgactgaa

SEQ ID NO: 86 - DNA, 27 bp, Artificial Sequence
  OligoDNA1-Asp-thiol
  modified_base (27)..(27): 3′-thiol
gacaggcaggtattctaaccgactgaa

SEQ ID NO: 87 - DNA, 26 bp, Artificial Sequence
  OligoDNA1-Glu-biotin
  modified_base (26)..(26): 3′-biotin-triethylene glycol (TEG)
gtgaaagggcggtgtcctgggcctct

SEQ ID NO: 88 - DNA, 30 bp, Artificial Sequence; identical to SEQ ID NO: 51
  FortRNAArg(CCU)depletion 3′-amine
  modified_base (30)..(30): 3′-amine

SEQ ID NO: 89 - DNA, 30 bp, Artificial Sequence; identical to SEQ ID NO: 52
  FortRNAArg(UCU)depletion
  modified_base (30)..(30): 3′-amine

SEQ ID NO: 90 - RNA, 19 nt, Artificial Sequence
  Figure 16 tRNA(GCU) KL stable quasi-continuous helix 5′ arm
gggguagcugcuaagugug

SEQ ID NO: 91 - RNA, 19 nt, Artificial Sequence
  Figure 16 tRNA(GCU) KL stable quasi-continuous helix 3′ arm
cacgcuuagcagauaccuc

(19) United States
(12) Patent Application Publication
(10) Pub. No.: US 2018/0171321 A1
(43) Pub. Date: Jun. 21, 2018

(54) PLATFORM FOR A NON-NATURAL AMINO ACID INCORPORATION INTO PROTEINS

(71) Applicant: THE UNIVERSITY OF QUEENSLAND, Queensland (AU)

(72) Inventors: Sergey MUREEV, Queensland (AU); Zhenling CUI, St Lucia (AU); Kirill ALEXANDROV, Queensland (AU)

(73) Assignee: THE UNIVERSITY OF QUEENSLAND

(21) Appl. No.: 15/561,867

(22) PCT Filed: Mar. 29, 2016
(86) PCT No.: PCT/AU2016/050239

§ 371 Date: Sep. 26, 2017

(30) Foreign Application Priority Data
Mar. 27, 2015 (AU) 2015901121

(51) Int. Cl. C12N 9/00; C12N 15/70; C12N 15/52; C40B 40/10; C07K 7/64

(52) U.S. Cl. CPC C12N 9/93; C12N 15/70; C12N 15/52; C12Y 603/04019; C07K 7/64; C12N 2310/10; C12N 2310/332; C40B 40/10

(57) ABSTRACT

A complement to tRNAs and a protein translation system are provided that enable the incorporation of non-natural moieties such as non-natural amino acids without compromising the ability to incorporate all of the twenty natural amino acids into the protein. This is achieved by reassigning one of the tRNA anticodons for amino acids that are normally decoded by at least two different tRNA anticodons to a non-natural moiety, wherein at least one codon can be uniquely recognized by the reassigned anticodon and at least one another codon from the same codon box cannot be recognized by the reassigned anticodon. Accordingly, an mRNA for translation is engineered to comprise one or more specific codons corresponding to the reassigned tRNA anticodons so that the non-natural moiety is incorporated into the translated protein at a selected position.

### CROSS REFERENCE TO RELATED APPLICATIONS

This application is a national stage application under 35 U.S.C. 371 and claims the benefit of PCT Application No. PCT/AU2016/050239 having an international filing date of 29 Mar. 2016, which designated the United States, which PCT application claimed the benefit of Australian Patent Application No. 2015901121 filed 27 Mar. 2015, the disclosure of each of which are incorporated herein by reference.

### REFERENCE TO SEQUENCE LISTING

This application contains a Sequence Listing submitted as an electronic text file named “Sequence_Listing_N409878WO_JRN_EGH.txt”, having a size in bytes of 28179 bytes, and created on Nov. 17, 2017. The information contained in this electronic file is hereby incorporated by reference in its entirety pursuant to 37 CFR § 1.52(e)(5).

### TECHNICAL FIELD

THIS INVENTION relates to protein engineering. More particularly, this invention relates to producing recombinant proteins that comprise one or more non-natural moieties (e.g non-natural amino acids) as well as any of all of the twenty (20) natural amino acids.

### BACKGROUND

Proteins are the central functional constituents in all living organisms. They are formed by natural biosynthetic pathways using genetically determined sequences of the twenty (20) genetically encoded “natural” amino acids that form the structural units of such proteins. To date, advances in understanding protein structure, function and diversity have largely focussed on natural structure, function and diversity given the relative simplicity of producing proteins that comprise natural amino acids. While the human genome encodes about 30,000 different proteins, there is potentially even more enormous diversity if proteins can be made that include structural moieties other than natural amino acids (e.g non-natural amino acids, chemical derivatives of natural amino acids, chemically-reactive moieties and the like). These “non-natural” proteins could be used to create synthetic protein libraries, protein-based bioactives such as pharmaceuticals, biotherapeutics (e.g antibodies, peptide hormones, immunomodulators), detection or diagnostic reagents having unprecedented structures and activities not found in naturally-occurring proteins. However, the recombinant production of such “non-natural” proteins has proven to be a difficult proposition.

Generally, approaches to producing recombinant “non-natural” proteins have relied upon manipulating protein translation (particularly in cell-free expression systems) by subverting the normal function of orthogonal tRNAs to provide a specific, natural amino acid for each step of protein chain elongation. It is possible to aminoacylate orthogonal tRNA with non-natural amino acids, either enzymatically or chemically. Enzymatic aminoacetylation requires the availability of orthogonal aminoacyl tRNA synthetase (aaRS). Typically, multiple rounds of directed evolution are required to generate orthogonal tRNA/aaRS pairs that can accept a non-natural amino acid as its substrate. While hundreds of non-natural amino acids have been incorporated into proteins using these approaches, the majority of them fall into a narrow structural category, defined by the specificity of their “parental” aaRS.

A noticeable departure from this concept is the use of an evolved tRNA acylation ribozyme (Flexizyme) that can charge non-natural amino acids onto tRNAs in vitro. This is achieved by the formation of a transient complex with the 3′-end of a tRNA, where the 3′-OH of the terminal ribose engages in a nucleophilic attack on the activated carboxyl carbon of the non-natural amino acid.

Protocols have also been developed for chemical acylation tRNAs. This enables the incorporation of chemical functionalities that are quite distinct from native amino acids and thereby difficult to charge via the aaRS route. While several synthesis routes have been developed, they are typically low-yield and are not widely used.

Other approaches have focussed on the incorporation of non-natural amino acids during protein translation by way of assigning tRNAs having orthogonal anticodons to encode non-natural amino acids. This also requires that a translated mRNA includes corresponding “reassigned” codons at positions where non-natural amino acids are to be incorporated. The most commonly used approach for the generation of orthogonal codons is “nonsense suppression”, which makes use of three stop codons in the genetic code: the amber, opal and ochre codons. In some organisms, stop codons can be read through and encode non-natural amino acids, such as the UGA codon for selenocysteine and the UAG codon for pyrrolysine. The main disadvantage of this system is the inherent reduction in protein expression yield resulting from the competition of release factors with orthogonal suppressor tRNAs.

Another approach is “frameshift suppression”. A natural frameshift suppressor tRNA containing an extended anticodon loop can read as a four-base codon, such as UAGN and ACCN, and suppress the +1 frame shift. However, encoding a non-natural amino acid via four-base codons is a far less efficient approach than the use of nonsense suppression, whereby the yield of a target protein is significantly lowered by false-reading of the quadruplet-codon as a triplet. A more recent strategy for amino acid incorporation is the exclusion of certain amino acids from the genetic code (such as Phe), thereby generating “free” codons that are assignable to a non-natural amino acid. However, this leads to a reduction of the amino acid vocabulary.

### SUMMARY

The present invention broadly provides a method whereby tRNA anticodons can be “reassigned” from natural amino acids to non-natural moieties, such as non-natural amino acids, to enable the production of recombinant proteins that include one or a plurality of non-natural moieties, without compromising the ability to incorporate natural amino acids into the recombinant protein.

In a first aspect, the invention provides method of producing a complement of tRNAs suitable for translation of a protein comprising at least one non-natural moiety, said method including the step of substituting at least one tRNA that comprises an anticodon for a natural amino acid with at least one tRNA comprising the same anticodon reassigned to a non-natural moiety, wherein the complement of tRNAs is operable to facilitate translation of an RNA which comprises a codon corresponding to the anticodon that has been reassigned to the non-natural moiety whereby the translated protein may comprise any or all of the twenty (20) natural amino acids.

Suitably, the reassigned anticodon is one of a plurality of different anticodons for the same natural amino acid.

In a second aspect, the invention provides a composition comprising a complement of tRNAs suitable for translation of a protein comprising at least one non-natural moiety, said complement comprising at least one tRNA that comprises an anticodon for a non-natural moiety reassigned from an anticodon for a natural amino acid, wherein the complement of tRNAs is operable to facilitate translation of an RNA which comprises a codon corresponding to the anticodon that has been reassigned to the non-natural moiety whereby the translated protein may comprise any or all of the twenty (20) natural amino acids.

Suitably, the reassigned anticodon is one of a plurality of different anticodons for the same natural amino acid.

In a third aspect, the invention provides a method of producing a translation system suitable for translation of a protein comprising at least one non-natural moiety, said method including producing a complement of tRNAs comprising at least one tRNA that comprises an anti-codon for a natural amino acid that has been reassigned to a non-natural moiety; and producing a transcribable RNA which comprises a codon corresponding to the anticodon that has been reassigned to the non-natural moiety, wherein the mRNA may be transcribed to produce a translated protein that may comprise any or all of the twenty (20) natural amino acids.

In a fourth aspect, the invention provides a translation system suitable for translation of a protein comprising at least one non-natural moiety, said system comprising: a complement of tRNAs comprising at least one tRNA that comprises an anticodon for a natural amino acid that has been reassigned to a non-natural moiety; and a translatable mRNA which comprises a codon corresponding to the anticodon that has been reassigned to a non-natural moiety, wherein the mRNA may be transcribed to produce a translated protein that may comprise any or all of the twenty (20) natural amino acids.

In a fifth aspect, the invention provides a method of producing a recombinant protein comprising at least one non-natural moiety, said method including the step of translating an mRNA which comprises a codon corresponding to an anticodon of a tRNA that has been reassigned to a non-natural moiety in a complement of tRNAs comprising at least one tRNA that comprises an anti-codon for a natural amino acid that has been reassigned to a non-natural moiety, wherein the translated protein may comprise any or all of the twenty (20) natural amino acids.

Suitably, according to the method, composition or system according to any of the aforementioned aspects, the reassigned anticodon is fourfold or six-fold degenerate.

Suitably, according to the method, composition or system according to any of the aforementioned aspects, the reassigned anticodon is an anticodon for Ile, Ala, Gly, Pro, Thr, Val, Arg, Leu or Ser.

In particular embodiments of the method, composition or system according to any of the aforementioned aspects, the translated protein may comprise one or a plurality of the same or different non-natural moieties.

In a sixth aspect, the invention provides a recombinant protein produced by the method of the fifth aspect.

It will be appreciated that isolated proteins may comprise one or a plurality of same or different non-natural moieties that facilitate PEGylation, conjugation of small molecules, labelling, immobilisation, intermolecular and/or intramolecular cross-linking or other interactions, formation of higher order structures and/or one or more catalytic activities, although without limitation thereto.

In an embodiment, the recombinant protein comprises two or more of the same or different non-natural moieties that are capable of intramolecular covalent bonding. In a particular embodiment, the recombinant protein is a macrocyclic protein.

Also provided is an mRNA molecule that encodes the recombinant protein of the sixth aspect.

It will be appreciated that the indefinite articles “a” and “an” are not to be read as singular indefinite articles or as otherwise excluding more than one or more than a single subject to which the indefinite article refers. For example, “a” protein includes one protein, one or more proteins or a plurality of proteins.

As used herein, unless the context requires otherwise, the words “comprise”, “comprises” and “comprising” will be understood to mean the inclusion of a stated integer or group of integers but not the exclusion of any other integer or group of integers.

### BRIEF DESCRIPTION OF THE FIGURES

FIG. 1. Denaturing PAGE analysis of 48 in vitro synthesized t7tRNA species. The t7tRNAs are denoted by the respective single letter amino acid code (uppercase) and the 5′-3′ anticodon triplet (lowercase). Polymorphic tRNA variants are indicated by the respective numerical indeces. tRNAs for Ile and Trp (Igau and Wcca) were generated through auto-cleavage of the HHRz-containing RNA precursor. The asterisks above and below the corresponding tRNA-bands denote the precursor and the excised HHRz, respectively. Initiator and elongator tRNAs(Met) are prefixed with “i” and “e”, respectively.

FIG. 2. Principle and calibration of the affinity clamp peptide biosensor. (A) Schematic representation of experimental procedure. The RNA sequences at the top represent coding frames for RGS-peptide and its derivatives, RGS1 and RGS2, the latter comprising the “insulator” codons (green) followed by the test-codon triplet (XXX, red). The resulting peptide containing the constant eight-amino-acid C-terminus and variable N-terminus binds to the biosensor composed of an autoinhibited TVMV-protease, with PDZ and FN3 domains forming the affinity clamp. Peptide binding results in a conformational change of the affinity clamp that in turn dislodges the inhibitory peptide from the active site of TVMV leading to protease activation and subsequent cleavage of the quenched reporter substrate. Q and F denote the fluorescence quencher and fluorophore groups, respectively. (B) The calibration curve for RGS-peptide obtained by assaying different concentrations of synthetic peptide in the heat-denatured E. coli extract. The initial rates were plotted against peptide concentration and the averaged data of triplicate experiments were fitted using regression coefficient (R2).

FIG. 3. Cell-free translation of eGFP and RGS-peptide in tRNA-depleted E. coli lysate. (A) Native tRNA-dependent eGFP expression. (B) Analysis of the depletion efficiency of native tRNAs for each codon assessed by withholding individual t7tRNA from the t7tRNA mixture, mediating RGS peptide translation in the depleted lysate. The codon/anticodon pairs corresponding to t7tRNA that were individually excluded are located below the corresponding bars. Elongator t7tRNAs were supplemented to a final concentration of 0.8 μM, with the initiator t7tRNAiMet at 1.6 μM and the native tRNA mixture at 1 μg/μl final concentrations.

FIG. 4. The t7tRNA decoding table. Ser, Arg, and Leu, shaded in blue, are encoded by mixed codon family boxes from which two codons (N1N2N3) belong to a split and the other four to unsplit codon family boxes¹⁹. The 4- and 2-fold degenerate amino acids are shaded in green and gray, respectively. Native tRNAs and t7tRNAs are denoted by their respective anticodons (N34N35N36). The letters other than A/U/G/C in the native tRNA anticodons denote modified nucleosides⁵⁵. The native and t7tRNA decoding patterns are indicated by arrow-lines from the left and right sides of the codon columns, respectively. Anticodons of the tRNAs specific for Lys, Glu, and Ile where modification either in anticodon or another part was essential for aminoacylation are highlighted in pink. Lys t7tRNA with U34 to C34 anticodon replacement based on tRNALys(UUU) is highlighted in red. The arrow-lines connecting t7tRNAs and the respective codons indicate the tRNA/codon combinations tested in the peptide biosensor assay. The dashed gray and black continuous arrow-lines correspond to <10% or ≥10% codon decoding efficiency, respectively. The associated number beside the black arrow-line indicates the calculated decoding efficiency of the t7tRNAs towards the analyzed codon as described in FIG. 11. The N34 modifications include “V”-uridine 5-oxyacetic acid, “[”-5-methyl-aminomethyluridine(mnm⁵U), “$”-5-carboxymethylaminomethyl-2-thiouridine(cmnm⁵ s²U), “S”-5-methylaminomethyl-2-thiouridine (mnm⁵s²U), “)”-5-carboxymethylaminomethyl-2′-O-methyluridine (cmnm⁵Um), “B”-2′-O-methylcytidine (Cm), “M”-N4-acetylcytidine (ac4C), “]”-2-lysidine (k2C), “I”-Inosine, “Q”-queuosine, and “Q*”-glutamyl-queuosine⁵⁵.

FIG. 5. Analysis of the purified native tRNAs for Ile, Glu and Asn. (A) Analysis of purified tRNAs on the denaturing PAGE stained with SYBR green. (B-D) The activities of purified native tRNAGlu (B), tRNAAsn (C) and tRNAIle(GAU) (D) analyzed by the peptide expression assay. The final concentration of native tRNAs in the assay was 1.6 μM.

FIG. 6. sGFP expression in semi-synthetic in vitro translation system. (A) DNA templates for three sGFP ORFs of various codon compositions were expressed in tRNA-depleted lysate programmed with semi-synthetic tRNA complements or native tRNA mixture at different concentrations. (B) Expression of sGFP_T2 template in tRNA-depleted lysate supplemented with semi-synthetic tRNA mixtures lacking the indicated t7tRNAs. Corresponding codons are shown below the tRNAs where y stands for U or C and r for A or G.

FIG. 7. Expression of sGFP_T1 in the PURE in vitro translation system and in the tRNA-depleted S30 lysate. In vitro translation experiments were performed without tRNA(circles), with native tRNA (squares) or with semi-synthetic tRNAs (triangles).

FIG. 8. Construction of DNA templates for in vitro synthesis of 48 E. coli tRNA species. (A) DNA templates for tRNAs starting with G, U or C; (B) DNA templates for tRNAs starting with A or U. These DNA templates contain a HHRz-coding sequence which auto-cleaves from the precursor after transcription releasing the tRNA. The box indicates the HHRz-coding sequence and the arrow in the box indicates the ribozyme cleavage site; (C) A summary of 48 E. coli tRNA species defined on the table from al to d12. The tRNA designations as per FIG. 1 and their respective gene copy number, the first base and size are shown.

FIG. 9. Calibration curves for RGS-peptide derivatives GGRGS (SEQ ID NO:1) and DDRGS (SEQ ID NO:2) in the affinity clamp assay. Here the initial rates of TVMV substrate de-quenching are plotted against the concentration of the peptide and fitted to a linear equation.

FIG. 10. The peptide expression levels from RGS templates prefaced by various codons. Codons were placed (A) immediately downstream the AUG initiator codon or (B) downstream the two consecutive GAU codons following AUG. The prefacing codons are shown below the respective bars.

FIG. 11. Calculation of native tRNA depletion efficiency and t7tRNA decoding activity. Two coding frames harbouring one or two test-codons were used. For the codons corresponding to 8 amino acids of the RGS-peptide, their synonymous variants were tested within the peptide-coding frame (shown in grey). In the context of two-codon template, 2 consecutive test-codons following GAU pair were used in addition to the same codon-variant in the RGS-coding frame. For other 12 amino acids not occurring in RGS-peptide sequence one or two test-codons were placed immediately downstream the pair of consecutive GAU codons. The “xxx” in red denotes the test-codon. In vitro translation reactions with different tRNA/codon combinations were carried out in the tRNA-depleted lysate. The reaction primed with E. coli native tRNAs (1 μg/μl final concentration) served as positive control (Σ native tRNA). The reaction primed with incomplete t7tRNA mixture lacking individual tRNAs (Σt7(−1) tRNA mix) was used to assess the depletion efficiency of native tRNA(s) for the given test-codon using the formula displayed in the box. The difference between the reactions primed with complete (Σ t7tRNA mix) and incomplete t7tRNA mixtures was used to assess the relative activity of test-codon specific t7tRNA as well as to reveal all possible cognate or non-cognate cross-recognition events within the unsplit or split codon family boxes. For one- and two-codon templates, the concentration of the test-codon specific t7tRNAs in the cell-free translation reaction was either 1.6 for both or 6.4 μM respectively. The full spectrum of codon/tRNA combinations for all 20 canonical amino acids was assayed and the peptide yield was determined by the affinity clamp assay. Unless indicated, each reaction was performed in duplicates.

FIG. 12. Evaluation of t7tRNA activities for Ser, Arg and Leu. A: Decoding efficiencies for the four t7tRNASer isoacceptors towards six Ser-codons were tested using one-codon template at 1.6 μM tRNA concentration (upper) or two-codon template at 1.6 μM (middle) and 6.4 μM (bottom) tRNA concentrations. B: The average t7tRNA decoding efficiencies towards the most of Ser, Arg and Leu codons were calculated as the mean value of relative activities provided by one- and two-codon templates at 1.6 μM or 6.4 μM of t7tRNA. The exception are the codons marked with the asterisks (CGU, CGC, CGA and CUG) the native isoacceptors for which were depleted incompletely and therefore masked the signal in one-codon template experiment. Therefore, t7tRNA functionalities towards these 4 codons were tested using two-codon template and their relative decoding activities were calculated as the mean value of activities at 1.6 and 6.4 μM of respective t7tRNA.

FIG. 13. Evaluation of t7tRNA decoding efficiencies for four-fold degenerate amino acids (Val, Pro, Thr, Ala and Gly). The codon-reading activities of Pro t7tRNA isoacceptors were calculated based on one-codon template reactions since the corresponding two-codon templates failed in translation. The average decoding activities for other codons marked with asterisk for which native isoacceptor was depleted only partially were calculated based on activities measured with two-codon template only.

FIG. 14. Analysis of t7tRNA decoding efficiencies for codons of 1-, 2- and 3-fold degenerate amino acids. The best-performing t7tRNA isoacceptors (see FIG. 8 for designation) as well as their relative activities are shown for each codon. The t7tRNA relative activities were calculated as in FIG. 13.

FIG. 15. PAGE-analysis of native tRNAGlu isolated on different oligonucleotide matrixes. Native tRNAGlu isoacceptors were purified by the NHS- and streptavidin-resins functionalized with OligoDNA1-Glu-amine and OligoDNA1-Glu-biotin, respectively, using two buffer systems (containing TMA⁺ or Na⁺)¹¹. The elution fractions (E1 and E2) were assessed on 8% denaturing PAGE for purity. The unbound tRNAs were removed by washing with 10 resin volumes of 10 mM Tris-HCl (pH7.5) buffer at room temperature until the absorbance of the eluate at 260 nm fell below 0.01 A260 units/ml. Target tRNA was eluted twice with 3 resin volumes of 10 mM Tris-HCl (pH7.5) at 68° C.

FIG. 16. Depletion of specific tRNAs from the total tRNA mixture using RNA aptamer (kissing loop) chromatography. (A) schematic representation of the base-pairing mechanism of kissing loop:tRNA complex formation (B) Flow chart for preparation of in vitro translation system depleted for a specific tRNA. C: Measurement of GFP-synthesis following tRNA reconstitution.

FIG. 17. ARS recoiling procedure.

FIG. 18. Results of ARS recoiling procedure on tRNA depletion.

FIG. 19. A: Cysteinylated or selemocysteinylated tRNACys(Secys) harbouring grafted GCU- or CCU-anticodons (Cys-tRNA(Cys)gcu/ccu). B: Effective suppression of single AGG-codon by Cys-tRNA(Cys)ccu in a context of translation reaction reconstituted of both all-tRNA depleted lysate and semi-synthetic tRNA complement lacking t7tRNA(Arg)ccu and primed with eGFP-coding template harbouring single AGG-codon C: Effective suppression of two consecutive AGC-codons in a translation reaction with the lysate directly depleted from endogenous tRNA(Ser)GCU using KL. D: Effective suppression of amber-codon in the eGFP-coding ORF (position 153) by pre-translationally selenocysteinylated tRNACys harbouring grafted CUA anticodon (reaction conditions as in B). E: Purification of Secys-tRNACys (CCU-anticodon) conjugation product with BodipyFL iodoacetamide.

FIG. 20. Macrocyclic peptide design by incorporation of non-natural amino acids. (A) Reassignment of AGG codon at the position 108 of EGFP using BODIPY-tRNA-Cys in reconstituted cell-free system lacking tRNA Arg (CCU). (B) Reassignment of AGC codon at position 4 of EGFP. Left panel is a scan of SDS-PAGE loaded with EGFP purified from E. coli in vitro translation reactions. Lane 1 represents the unmodified cell-free system while lanes 2 and 3 represent the reaction in which GcutRNA was replaced with Pyr GcutRNA charged with cyclooctene Lysine (COC). The COC was labelled with Atto488-tetrazine prior to purification. (C) Expression yields of EGFP with one or two reassigned Ser(AGC) or amber (TAG) codons. (D) Tetrazine ligation reaction (E) Structures of bifunctional tetrazine derivative capable of ligating two cyclooctene containing amino acids. (F) Trifunctional tetrazine derivative (G) SDS-PAGE analysis of in vitro translation mixture expressing COC-containing EGFP protein (B) cross-linked with di-tetrazine (DT). The gel was loaded with unboiled samples and scanned for EGFP fluorescence (H) Example of macrocyclic polypeptides formation using codon reassignment and copper catalysed click reaction.

FIG. 21. Translation efficiency of two GFP-coding templates with either all six (6) or just one arginine codon/s changed to AGG (6AGG or 1AGG on the figure, respectively) in the translation reactions programmed with 3 tRNA mixtures as indicated. Total tRNA and Depl tRNA indicate the total home-isolated tRNA mixture before and after tRNAccu/ucu depletion, respectively. A third mixture contains synthetic t7tRNAccu to added a final concentration at of 5 μM.

FIG. 22. Comparative analysis of amber codon suppression efficiencies in E. coli cell-free translation system with different nnAA/o-tRNA/aaRS combinations. A: The structure of nnAAs used here. B-D: Suppression Amber suppression of A_151X template. The suppression efficiency is defined as a ratio compared to the fluorescence of percentage of wild type eGFP fluorescence wild type eGFP without stop codon inside its ORF. B: Incorporation of four nnAAs by three PylRS variants. The final concentrations of PylTcua, PylRS variants and nnAAs were 20 μM, 20 μM and 1 mM, respectively. C: AzF incorporation by four MjY tRNA variants. MjY1, MjY2, MjY3 and MjY4 are corresponding to the tRNAs pAzPhe1, pAzPhe2, pAzPhe3 and tRNAopt CUA in original publication^{35,37}. The final concentrations of MjYtRNA variants, AzFRS and AzF are 10 μM, 10 μM and 1 mM, respectively. D: Effect of o-tRNA and o-aaRS concentrations on amber codon suppression efficiency of MjY2/AzFRS/AzF and PylT/PylRSAF/PrK. E: The effect of codon contexts on nnAA incorporation efficiency in two orthogonal systems. The concentration of MjY2 and AzFRS were 10 μM for both while PylT and PylRS were used at 20 and 30 μM, respectively. Four templates, named A_1X, A_151X, B_1X and B_151X, were used in the analysis (sequences are shown in SI). A and B denote the template backbones with different codon biases where codon vocabulary in A is optimized for eGFP while in B it is simplified mostly utilizing the unique codon to decode each amino acid type. The numeric indicates the position of amber codon in ORF sequence. The relative activity in individual reactions is given as a percent of fluorescent intensity provided while obtained when using EctRNATyr(cua) as a suppressor.

FIG. 23. AGG reassignment to AzF in the context of four GFP ORFs. The GFP protein was expressed in cell-free system depleted of tRNA species decoding AGG codon with and without addition of MjY suppression system (MjY2/AzFRS). The final concentrations for MjY2, AzFRS, AzF are were 10 μM, 10 μM and 1 mM, respectively. The anticodon in MjY2 was changed to CCU in order to reassign AGG codon to AzF.

FIG. 24. Synthesis of BPFL-tRNA for AGG and UAG-suppression. (A): HPLC purification of BPFL-conjugated tRNAs. The tRNACys bearing CUA or CCU anticodons were charged with Cys by CysRS and reacted with the iodoacetamide group on the BP-FL. Absorbance measurement at 254 nm detected the ribonucleic acid while 490 nm channel was used for BP-FL detection. (B-C): Analysis of labelled protein yields by fluorescence scanning and total protein yield by Western blotting of 12% PAGE gel. The BP-FL incorporation was detected on the gel after quenching eliminating the GFP fluorescence by boiling of the sample. The total protein was detected by Western bolting using anti-GFP antibody. The intensity of each band was calculated by ImagJ scaled to by the intensity of the faintest band that was set to 1. (B) The sGFP_T2 template harboring one AGG codon was expressed with or without addition of Biodipy-charged tRNAccu in the normal lysate or tRNA depleted lysate supplemented with indicated tRNA mixtures. (C) Comparison of BP-FL incorporation mediated through amber or AGG codon suppression. The translation reactions for AGG suppression are programmed by templates with single AGG at the 1st or 151st GFP ORF respectively and reconstituted of tRNA-depleted lysate and total tRNA mixture lacking AGG suppressors with or without BPFL-tRNAccu suppressors. The similar reactions are performed for amber suppression but with total tRNA mixture, template harbouring harboring single amber codon and BPFL-tRNAcua suppressor.

FIG. 25. Site-specific double labelled labelling of CaM for smFRET-based conformational change analysis. (A) Fluorescence scanning scans of mono- and dual-labelled CaM. Dual labelled CaM protein was prepared in the cell-free translation system reconstituted of tRNA-depleted lysate and total tRNA mixture lacking AGG suppressors. Two suppression systems, MjY2(cua)/AzFRS/AzF and BPFL-cys-tRNAccu, were supplemented for recoding UAG and AGG codon at the 1st and 149th position, respectively. AzF incorporated in the protein was then reacted with DIBO-TAMRA through copper-free click chemistry. Single labelled CaM proteins were prepared the same way but using only one suppression system while the other o-codon was decoded either by tRNATyrcua or tRNAArgccu. SDS-PAGE gel loaded with single and double labelled CaM was scanned using two chanelschannels as indicated. (B) Fluorescence emission spectra of single-labelled and double labelled CaM excited at 488 nm. The concentration of TAMRA-labelled protein was adjusted to make sure its emission fluorescence was the same as that of dual labelled protein when excited at 543 nm. (C): Structural representation of CaM in Ca2+-free (PDB: 1CFC) and Ca2+-binding form (PDB: 4CLN). The green and red dots indicate the BP-FL and TAMRA installed in the CaM ORF at the 1st and 149th position, respectively. (D-E) smFRET histograms recorded of dual-labelled CaM under different conditions. (D): in 50 mM Tris-HCl, 150 mM NaCl buffer without Ca2+, (E): 2 mM Ca2+, (F): with 10 mM EDTA. The solid lines represent Gaussian fits to data using Origin software. Each peak indicates an individual conformation.

FIG. 26. Vector map for pOPINE_CaM template.

FIG. 27. Translational performance of depleted commercial tRNA mixture with or without supplementation of t7tRNAccu. Two GFP-coding templates with either all or just one AGG codon, 6AGG or 1AGG indicated in the figure, were tested in the translation reactions programmed with 3 tRNA mixtures. Total tRNA and Depl tRNA indicate the commercial tRNA mixture before and after tRNAccu/ucu depletion. A third mixture contains t7tRNAccu to make a final concentration at 5 μM.

FIG. 28. Alignment of four previously reported MjYtRNA species. MjY1, MjY2, MjY3 and MjY4 are corresponding to the tRNAs named pAzPhe1, pAzPhe2, pAzPhe3³ and tRNAopt CUA in original publications⁴. Unlike the sequence reported in the original paper, the N63 to N67 in the MjY2 tRNA here is “CATCG” instead of “CATCGT” (the “T” in the end appears to be erroneous in the original paper).

FIG. 29. The incorporation efficiency of 5 nnAAs precharged by flexizyme with their respective active group. A: The structure of five activated acid substrates for tRNA acylation by Flexizyme, including L-Propargylglycine 4-chlorobenzyl thioester (Pra-CBT), L-Azidolysine 4-chlorobenzyl thioester (Lys(N3)-CBT), L-Azidohomoalanine 4-chlorobenzyl thioester (Aha-CBT), NE-biotinyl-L-lysine 3,5-dinitrobenzyl ester (Lys(biotin)-DBN) and L-Azidophenylalanine-cyanomethyl ester (AzF-CME). The nnAAs are shown in black while their respective active groups in red. B: The amber suppression efficiency of 5 nnAAs precharged tRNA tested by using A_151X template in the cell free translation system. The precharged tRNA was used to prime the translational reactions at 10, 20 or 30 uM of final tRNA concentration. The flexizyme reaction time and the final concentration of precharged nnAA-tRNAs were indicated. Among the five nnAAs tested here, AzF demonstrated the highest translational activity. This suggested that compared to other nnAAs, the flexizyme could charge AzF very well on the o-tRNAs and this precharged AzF-tRNA was well accepted by EF-Tu and Ribosome.

FIG. 30. Orthogonality of the two suppression systems, MjY2/AzFRS/AzF and PylT/PylRSAF/PrK, in E. coli in vitro translation systems. The reactions lacking either nnAA-substrate, o-tRNAs or enzyme were used to assess the level of non-specific suppression on amber codon in template A_151X. The concentration of MjY2 and AzFRS were 10 μM for both while PylT and PylRS were used at 20 and 40 μM, respectively.

FIG. 31. (A) The TAG codon in Template A_151X was reassigned to AzF. The MS/MS analysis of the identified peptides containing the nnAA incorporation site are shown. (B). The AGG codon in Template A_151R was reassigned to AzF. The MS/MS analysis of the identified peptides containing the nnAA incorporation site are shown. (C) The AGG codon in Template A_1R was reassigned to AzF. The MS/MS analysis of the identified peptides containing the nnAA incorporation site are shown.

FIG. 32. Structure of BPFL-tRNACys(CCU)

FIG. 33. The tertiary complex of tRNA, amino acid and elongation factor. The structure of tRNATrp charged with different amino acids formed tertiary complex with elongation factor EF-Tu. A: Trp-tRNA-EFTu, B: BPFL-cys-tRNA-EFTu.

FIG. 34. The ability of tRNACysccu in supporting AGG translation. Cell free translation reactions were performed in the context of tRNA-depleted lysate supplemented with semisynthetic tRNA mixtures lacking AGG-isoacceptors. The sGFP_T2 template harboring one AGG codon was expressed with or without addition of tRNA species with CCU anticodon as indicated. tRNAArgccu is the synthetic wt AGG tRNA isoacceptor and severs as a positive control.

FIG. 35. Translational performance of commercial tRNA mixture used at 0.25 and 0.5 μg/μl depleted for native AGC- and AGG-codon tRNA supressors (Σ-a5/6,-c11) with or without supplementation of their t7tRNA counterparts: t7tRNAccu (a05) and t7tRNAgcu (c11). Two GFP-coding templates with either just one unique (eGFP ×1AGC) or two consecutive (×2AGC) AGC codons at positions 4 or 4,5 both devoid of any of AGG and one template with both unique AGC (position 4) and AGG (position 151) codons (eGFP ×1AGC/×1AGG) were used in the suppression experiments. For each template the translation reactions was programmed with either total tRNA mixtures lacking native isoacceptors for the desired AGC- and AGG-codons or the same mixture containing the supplemented t7tRNA analogs for both. Schematic representation of the used ORF is shown below each graph.

FIG. 36. Experimental work-flow for double-sense codon labelling. CaM native ORF is shown in black, precision protease cleavage site is denoted by blue circle while affinity clam binding petidic tag is shown as brown hexagonal. ×1 and ×2 indicate single or double-consecutive AGC-codons at the 1^{st} or 1^{st} and 2^{nd} positions of CaM, respectively. Low case indicates the bias for the rest of Ser and Arg codons.

FIG. 37. Site-specific double labelling of CaM. Fluorescence scanning of mono- and dual-labelled CaM. Dual labelled CaM protein was prepared in the cell-free translation system reconstituted of tRNA-depleted lysate and total tRNA mixture lacking suppressors for both AGG/AGA and AGC-codons. Two suppression systems, MjY2(gcu)/AzFRS/AzF and BPFL-cys-tRNAccu, were supplemented for decoding AGC and AGG codon at the 1st and 151st position, respectively. Protein harbouring AzF was either loaded on SDS-PAGE directly or subjected to the subsequent conjugation with DIBO-TAMRA through the copper-free strain-promoted cycloaddition. The two CaM-coding ORFs harbouring 1 (×1AGC) or two (×2) consecutive AGC-codons at position 1 or 1,2-in addition to a unique AGG at the 151 position were used. SDS-PAGE gel was scanned in two indicated wavelengths using two channels.

### DETAILED DESCRIPTION

The present invention provides a protein translation system that allows the incorporation of non-natural moieties (e.g non-natural amino acids) into the translated protein without compromising the ability to incorporate all of the twenty (20) natural amino acids into the protein. This is achieved by reassigning one of the tRNA anticodons for amino acids that are normally decoded by at least two (2) different tRNA anticodons to a non-natural moiety, wherein at least one codon can be uniquely recognized by the reassigned anticodon and at least one another codon (i.e. from the same codon box) cannot be recognized by the reassigned anticodon. Accordingly, an mRNA for translation is engineered to comprise one or more specific codons corresponding to the reassigned tRNA anticodon(s) so that the non-natural moiety is incorporated into the translated protein at an appropriate or desired position.

In an aspect, the invention provides a method of producing a complement of tRNAs suitable for translation of a protein comprising at least one non-natural moiety, said method including the step of substituting at least one tRNA that comprises an anticodon for a natural amino acid with at least one tRNA comprising the same anticodon reassigned to a non-natural moiety, wherein the complement of tRNAs is operable to facilitate translation of an RNA which comprises a codon corresponding to the anticodon that has been reassigned to the non-natural moiety whereby the translated protein may comprise any or all of the twenty (20) natural amino acids.

In another aspect, the invention provides a composition comprising a complement of tRNAs suitable for translation of a protein comprising at least one non-natural moiety, said complement comprising at least one tRNA that comprises an anticodon for a non-natural moiety reassigned from an anticodon for a natural amino acid, wherein the complement of tRNAs is operable to facilitate translation of an RNA which comprises a codon corresponding to the anticodon that has been reassigned to the non-natural moiety whereby the translated protein may comprise any or all of the twenty (20) natural amino acids.

As generally used herein, a protein is a polymer comprising two (2) or more covalently linked moieties which may be natural L-amino acids and/or non-natural moieties such as non-natural amino acids. Typically, a peptide is a protein comprising no more than fifty (50) contiguous moieties. Typically a polypeptide is a protein comprising more than fifty (50) contiguous moieties.

As used herein, a “natural amino acid” is an L-amino acid that can be genetically encoded by a genome of an organism. Typically, a natural amino acid reacts at a peptidyl transferase center at a physiological range of kinetic constants. Preferably, the natural amino acid is selected from the group consisting of: alanine, asparagine, aspartate, cysteine, glycine, lysine, glutamate, glutamine, arginine, histidine, methionine, serine, threonine, valine, leucine, isoleucine, proline, tyrosine, tryptophan, phenylalanine, or a derivative of these which would normally be incorporable into a translated protein via a tRNA having an anticodon for any of these natural amino acids. Derivatives may be naturally-occurring derivatives such as post-translationally modified amino acids (e.g. selenocysteine).

As used herein, a “non-natural moiety” may be any molecule capable of incorporation into a protein translatable from an RNA template via ribosome-mediated chain elongation, with the proviso that it is not a natural amino acid as hereinbefore defined. Generally, such non-natural moieties may include non-natural amino acids, natural or synthetic chemical derivatives of natural amino acids and/or chemically-reactive moieties such as moieties capable of forming intramolecular covalent bonds. In some embodiments, subject to the above proviso, the non-natural amino acid may be any organic compound with an amine (—NH₂) and a carboxylic acid (—COOH) that is capable of peptide bond formation. Non-limiting examples of non-natural moieties include D-amino acids, selenocysteine, pyrrolysine, N-formylmethionine, α-Amino-n-butyric acid, norvaline, norleucine, alloisoleucine, t-leucine, α-Amino-n-heptanoic acid, pipecolic acid, α,β-diaminopropionic acid, α,γ-diaminobutyric acid, ornithine, allothreonine, homocysteine, homoserine, β-alanine, β-amino-n-butyric acid, β-aminoisobutyric acid, γ-aminobutyric acid, α-aminoisobutyric acid, isovaline, sarcosine, N-ethyl glycine, N-propyl glycine, N-isopropyl glycine, N-methyl alanine, N-ethyl alanine, N-methyl β-alanine, N-ethyl β-alanine.

Chemical moieties that may be suitable for the formation of intramolecular covalent bonds may be any which facilitate: incorporation of two identical reactive groups that undergo homo-condensation; incorporation of two identical reactive groups that undergo condensation via bifunctional reactive groups; or incorporation of two different reactive groups that undergo hetero-condensation. Non-limiting examples include amino acid side chains or terminal amines or carboxylic acids modified by moieties such as NHS-esters, maleimidies, haloacetyls, although without limitation thereto. Reference may also be made to Chapters 14 and 15 Of Current Protocols in Protein Science Eds. Coligan et al (2001-2012) for a more detailed discussion of chemical modification of amino acids and proteins.

As generally used herein, a “tRNA” is a transfer RNA or isoacceptor RNA molecule inclusive of native and synthetic tRNA molecules. Typically, a tRNA molecule comprises a nucleotide sequence of about 60-93 nucleotides with regions of internal base pairing that results in four or five double-stranded stems and three or four single-stranded loops formed from the primary structure. The 5′ and 3′ termini are located at the termini of an internally base paired “acceptor stem”. The 3′ terminus comprises the nucleotide sequence CCA, the terminal “A” nucleotide being the site of aminoacylation by amino acid-specific aminoacyl tRNA synthases. The amino acid-specific anticodon is located in Loop II.

As used herein an “anticodon” is a nucleotide sequence of a tRNA molecule which corresponds to a codon in an mRNA, or its DNA precursor, that encodes a natural amino acid or serves as a translation terminator (UAA, UGA, UAG). In many cases the anticodon facilitates aminoacylation of the tRNA by the appropriate aminoacyl tRNA synthase with the correct amino acid encoded by the translatable mRNA codon to which the anticodon corresponds (except for Ser, Ala and in some cases, Leu). In this context, the anticodon may be a 3′-5′ tri-nucleotide sequence that forms Watson-Crick base pairs at least at the first and second positions with the corresponding 5′-3′ mRNA codon sequence. Since the genetic code is degenerate there may be more than one tRNA decoding a codon box for a given amino acid. Such tRNAs decoding the same amino acid and comprising different anticodons are defined as “isoacceptors”. In the case of all codons, except those encoding Met and Trp, the specificity of base-pairing between the anticodon and the mRNA codon is defined by the first two nucleotides (i.e read 3′-5′ in the anticodon and 5′-3′ in the mRNA codon), such that the same anticodon may correspond to two or more degenerate mRNA codons. Each of the single tRNA-isoacceptors for Asp, Asn, Cys, Glu, Gln, His, Lys, Phe and Tyr correspond to two different mRNA codons (i.e “two-fold degenerate”). Ile has two isoacceptors, one of which corresponds to a single mRNA codon (i.e “three-fold degenerate”). There are two isoacceptors for Ala, Gly, Pro, Thr and Val that correspond to respective codons in an RNA template (i.e “four-fold degenerate”). For Arg, Leu and Ser there are four or five isoacceptors that correspond to respective codons (i.e “six-fold degenerate”).

Suitably, the composition disclosed herein includes at least one tRNA that comprises an anticodon reassigned from a natural amino acid to a non-natural moiety. In this context “reassigned” means that the anticodon no longer corresponds to an RNA or DNA codon that encodes its orthogonal, natural amino acid. To achieve this, any RNA translatable using the composition also comprises a corresponding codon at a position where it is intended to incorporate the non-natural moiety into the synthesized protein. By utilizing anticodons that correspond to redundant codons, the composition enables incorporation of non-natural moieties without compromising the ability to include all twenty (20) natural amino acids in a translated protein.

In principle, a reassigned anticodon according to the invention may be any that corresponds to a redundant RNA codon encoding a natural amino acid, wherein the reassigned codon is one of a plurality of different codons for the same amino acid. Suitably, amongst the redundant codons for a given amino acid there is at least one RNA codon which uniquely corresponds to the reassigned anticodon and at least one RNA codon which does not correspond to the reassigned anticodon. As will be understood by persons skilled in the art, the genetic code provides degeneracy whereby all natural amino acids other than tryptophan and methionine are encoded by more than one codon. A preferred object of the invention is to provide a protein translation system where, notwithstanding the incorporation of non-natural moieties into the protein, the ability to incorporate all of the twenty (20) non-natural amino acids is retained. Accordingly, it is preferred that the reassigned anticodons are four-fold degenerate or six-fold degenerate, as hereinbefore described. This allows a “non-reassigned” anticodon to perform its normal role in incorporating a natural amino acid during protein translation. In a particularly preferred form, the anticodons are for Leu, Arg or Ser.

It will also be appreciated from the Examples that within each pool or complement of potentially reassignable anticodons for any particular amino acid, there may be rules or criteria that apply to the selection of appropriate anticodons for reassignment. These may include the effect of “wobble” in base-pairing, the potential for cross-recognition between tRNA anticodons and mRNA codons and/or suitability of preparing synthetic tRNA molecules comprising the reassigned anticodon. A more detailed examination of these factors is provided in the Examples.

In one embodiment, the method of producing the composition includes the steps of: (i) depleting one or more tRNAs from a complement of tRNAs suitable for translation of a protein comprising natural amino acids; and (ii) reconstituting the depleted complement of tRNAs with one or more tRNAs respectively reassigned to non-natural moieties and respectively coupled to the non-natural moieties.

In some embodiments, the complement of tRNAs in (i) is present in a cell-free translation system.

In one embodiment of step (i), substantially all tRNAs for natural amino acids are depleted from the complement of tRNAs. In a particular embodiment, depletion is by way of binding the tRNAs to ethanolamine sepharose.

In another embodiment of step (i), one or more tRNAs for natural amino acids are selectively depleted from the complement of tRNAs. In an embodiment, selective depletion is by selectively binding the one or more tRNAs to respective, specific tRNA depleting agents. Such agents may be a natural or synthetic protein, DNA, PNA etc.

In one embodiment, the tRNA depleting agent is an RNA aptamer. Suitably, each RNA aptamer forms a specific, high affinity complex with a particular tRNA. In an embodiment, the RNA aptamer comprises a nucleotide sequence which forms a high affinity complex by binding to the anticodon-containing nucleotide sequence of the target tRNA. In some embodiments, the RNA aptamer may be referred to as a “kissing loop” RNA.

In yet another embodiment, the tRNA depleting agent comprises one or plurality of single-stranded DNA oligonucleotides having nucleotide sequences specific for respective tRNAs. This embodiment is particularly useful for depletion of specific tRNAs.

In one embodiment of step (ii), the reconstituting tRNAs may comprise synthetic tRNAs, native tRNAs, or mixtures thereof (referred to herein as a “semi-synthetic tRNA complement”). Synthetic tRNAs may be made by any chemical or enzymatic method known in the art inclusive of RNA polymerase-mediated synthesis. Non-limiting examples include, SP6, SP3 and T7-mediated synthesis, although without limitation thereto. As will be described in more detail hereinafter, synthetic tRNAs for asparagine, glutamate and isoleucine are substantially non-functional. Accordingly, for the purposes of reconstitution, tRNAs for Asn, Glu and Ile are suitably native tRNAs, although may be substituted by engineered tRNA mutants.

In some embodiments, specific tRNAs may be obtained and used for reconstitution after selectively binding to an RNA aptamer such as a “kissing loop” RNA as hereinbefore described.

Non-natural moieties may be coupled, charged or loaded onto the reconstituting tRNA by any method known in the art. These may include use of chemical aminocylation or enzymatic aminoacylation. Non-limiting examples of enzymatic aminoacylation include the use of natural, modified aminoacyl tRNA synthases such as PylRS or variants thereof used in pyrrolysine tRNA synthase-mediated aminoacylation, Methanococcus jannaschii tyrosyl-transfer RNA synthetase (Mj TyrRS) or variants thereof, Flexizyme-mediated aminoacylation and/or aminoacylation by a cysteinyl tRNA synthase.

In embodiments relating to aminoacylation of a tRNA with a non-natural moiety by a cysteinyl tRNA synthase, a preferred embodiment provides in vitro charging of a synthetic tRNA having an anticodon reassigned to the non-natural moiety. Mutation of a wild-type cysteine tRNA anticodon reduces the affinity of the cysteinyl-tRNA synthetase for the cysteine tRNA but does not substantially reduce or inhibit the aminoacylation activity of cysteinyl tRNA synthetase in vitro where the high concentrations of reactants compensate for the reduction in affinity. The cysteine tRNA anticodon mutants are unable to be aminoacylated with cysteine in vivo (e.g in a cell-free translation system) due to the typically lower level of cysteinyl-tRNA synthetase and the presence of competing endogenous cysteine tRNAs. Accordingly, a complement of tRNAs may be produced by charging the synthetic tRNA having an anticodon reassigned to the non-natural moiety in vitro and reconstituting the tRNA complement with this synthetic tRNA, or a plurality of different tRNAs each comprising a different non-natural moiety. As will be appreciated from the foregoing, the composition disclosed herein may be suitable for use in a method or system for recombinant protein production.

Accordingly, an aspect of the invention provides a method of producing a translation system suitable for translation of a protein comprising at least one non-natural moiety, said method including producing a complement of tRNAs comprising at least one tRNA that comprises an anti-codon for a natural amino acid that has been reassigned to a non-natural moiety; and producing a transcribable RNA which comprises a codon corresponding to the anticodon that has been reassigned to the non-natural moiety, wherein the mRNA may be transcribed to produce a translated protein that may comprise any or all of the twenty (20) natural amino acids.

Another aspect of the invention provides a translation system suitable for translation of a protein comprising at least one non-natural moiety, said system comprising: a complement of tRNAs comprising at least one tRNA that comprises an anticodon for a natural amino acid that has been reassigned to a non-natural moiety; and a translatable mRNA which comprises a codon corresponding to the anticodon that has been reassigned to a non-natural moiety, wherein the mRNA may be transcribed to produce a translated protein that may comprise any or all of the twenty (20) natural amino acids.

A further aspect of the invention provides a method of producing a recombinant protein comprising at least one non-natural moiety, said method including the step of translating an mRNA which comprises a codon corresponding to an anticodon of a tRNA that has been reassigned to a non-natural moiety in a complement of tRNAs comprising at least one tRNA that comprises an anti-codon for a natural amino acid that has been reassigned to a non-natural moiety, wherein the translated protein that may comprise any or all of the twenty (20) natural amino acids.

Suitably, the system and method of these aspects is for performing protein translation in vitro or an otherwise acellular or cell-free translation system. Non-limiting examples include wheat germ, insect, HeLa lysate, rabbit reticulocyte lysate, E. coli and Leishmania-based systems.

As will be understood from the foregoing, the invention is at least partly predicated on reassigning one or a plurality of redundant codons of the genetic code to encode non-natural moieties. Accordingly, the invention provides a method whereby a translatable RNA may be produced which comprises one or more codons that selectively correspond to anticodons of respective tRNAs that have been reassigned from natural amino acids to non-natural moieties.

A particular advantage of the present invention is that the translatable mRNA may comprise codons for any or all of the twenty (20) natural amino acids in addition to the one or more non-natural moieties, whereby a translated protein may comprise any or all of the twenty (20) natural amino acids in addition to the one or more non-natural moieties.

Accordingly, an aspect of the invention provides a recombinant protein produced by the method disclosed herein.

Also provided is an mRNA molecule that encodes the recombinant protein disclosed herein.

It will be appreciated that isolated proteins may comprise one or a plurality of same or different non-natural moieties that facilitate further derivatisation such as PEGylation, conjugation of small molecules, labelling, tagging, immobilisation, intermolecular and/or intramolecular cross-linking or other interactions, formation of higher order structures and/or one or more catalytic activities, although without limitation thereto.

In a particular embodiment, the translated protein may comprise one or more non-natural moieties that facilitate the formation of one or more intramolecular covalent bonds. In a particular embodiment, the recombinant protein is a macrocyclic protein.

By way of example, cyclic polypeptides may be useful in bioinformatics methods to design focused peptide libraries containing representatives from some or all of the structural fold classes found in nature.

The availability of multiple reassigned codons in the production of proteins comprising cyclizable amino acids may facilitate the construction of libraries of macrocyclic peptides. To form covalent intramolecular bonds, the following approaches may be used: a) incorporation of two identical reactive groups that undergo homo-condensation; b) incorporation of two identical reactive groups and their condensation via bifunctional reactive groups; and c) incorporation of two different reactive groups that undergo hetero-condensation. As described herein, reassignment of tRNA anticodons provides a system for testing these approaches. A non-limiting example of constructing a test mRNA coding for a synthetic 10-mer peptide carrying Ser and Arg codons and a C-terminal affinity clamp tag will be described in more detail in the Examples.

It will be appreciated that the invention disclosed herein may be applicable to the production of any protein or peptide, or library comprising a plurality of different proteins and peptides, that incorporate moieties other than natural amino acids in targeted or selected locations, while allowing for the incorporation of natural amino acids as desired.

So that the invention may be fully understood and put into practical effect, reference is made to the following non-limiting Examples.

### EXAMPLES

### Example 1

### Development of a Semi-Synthetic Protein Translation System

### Materials and Methods

### Peptide Biosensor (Affinity Clamp) Assay

The reporter peptides of different sequences, RGSIDTWV (SEQ ID NO:3), GGRGSIDTWV (SEQ ID NO:4), DDRGSIDTWV (SEQ ID NO:5), and the fluorescently quenched TVMV substrate peptide (5-Amino-2-nitrobenzoic acid-ETVRFQSK-7-Methoxycoumarin-4-yl (SEQ ID NO:6)), were synthesized by Mimotopes. A fusion of autoinhibited protease and the affinity clamp (peptide biosensor) was purified by Ni2+-NTA affinity chromatography and stored in 50 mM Tris-HCl, 1 M NaCl, 5 mM EDTA, 2 mM TCEP, ans 10% glycerol buffer (pH 8.0).

Typically the affinity clamp assay was carried out in buffer A of 50 mM Tris-HCl, 1 M NaCl, 1 mM DTT, and 0.5 mM EDTA (pH8.0), supplemented with 1.3 μM of peptide biosensor and 300 μM of TVMV substrate peptide. RGS-peptides were used either as a solution in the buffer or in the context of in vitro translation, and the reaction progress was monitored by exciting the sample at 330 nm and recording the fluorescence changes at 405 nm for 1 h using the Synergy plate reader. A calibration plot was generated to establish the relationship between initial rates of substrate cleavage (Vmax) and known concentrations of the control peptide. Samples were assayed in triplicate.

To quantify the RGS peptide and its derivatives in a cell-free translation reaction, the S30 E. coli cell extract formulated for coupled transcription-translation and supplemented with ×2 protease inhibitor cocktail (Roche) was primed with the desired peptide-coding DNA template and incubated at 32° C. for 1 h. After translation, NaCl was added to the reaction mixture to the final concentration of 1 M followed by incubation at 65° C. for 10 min to inactivate the endogenous proteases otherwise competing with TVMV-protease for the substrate peptide. After centrifugation at 10000 rpm for 5 min, 10 μl of supernatant was used for the affinity clamp assay as described above. In the context of in vitro translation reactions, the calibration plot was obtained by supplementing different amounts of synthetic peptides into the cell-free translation reaction lacking the DNA template.

Preparation of tRNA-Depleted Lysate

The s30 E. coli extract was prepared from BL21(DE3)GOLD as described in 27, and stored frozen in 10 mM Tris-Acetate (pH8.2), 14 mM Mg(OAc)2, 0.6 mM KOAc, and 0.5 mM DTT buffer at −800 before the tRNA-depletion procedure. For tRNA depletion, 2.5 ml of s30 extract was rebuffered on an NAP-25 column (GE healthcare) equilibrated with buffer B of 25 mM KCl, 10 mM NaCl, 1.1 mM Mg(OAc)2, 0.1 mM EDTA, 10 mM Hepes-KOH(pH7.5), 1 mM Mg(OAc)2), and 120 mM KOAc. After re-buffering, the lysate was incubated with 0.8-1.2 ml settled ethanola-mine-Sepharose matrix, prepared according to previous procedures 28, at 40 C for 30 min on an orbital shaker. Following the incubation, the supernatant was collected and the matrix was washed with 1 ml of buffer B containing 180 mM KOAc. The flow-through was combined with the supernatant from the previous step to yield the tRNA-depleted lysate, snap frozen, and stored at −800.

The cell-free translation reactions in the S30 lysate were performed at 320 following the standard protocol 27 using 30 nM DNA template and Mg(OAc)2 at 10 mM final concentration. The PURExpress® Δ (aa, tRNA) Kit (E6840S), was purchased from NEB and used according to the manufacturer's instructions.

Construction of tDNAs and Plasmids for Peptide and sGFP Expression

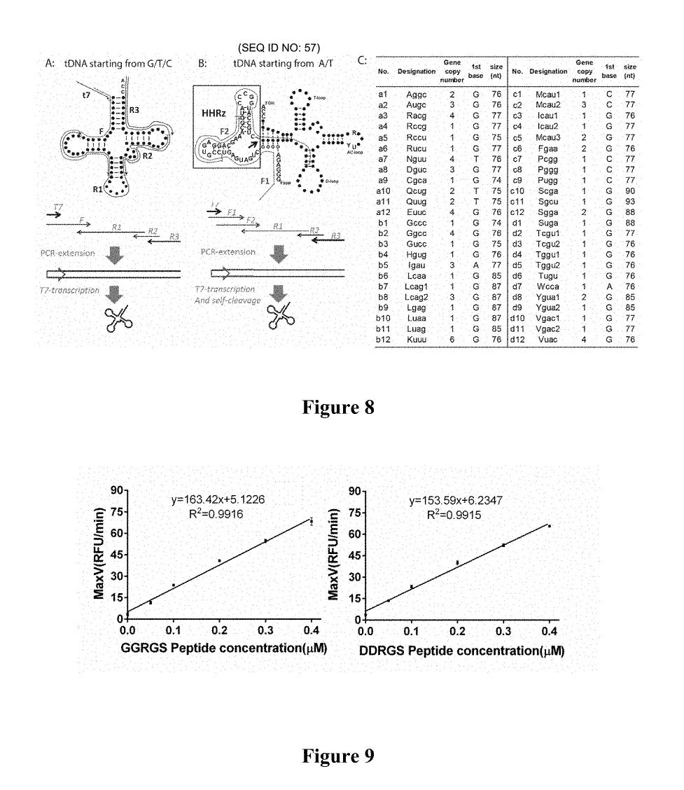

The coding sequences for tRNAs were obtained from the Genomic tRNA database (GtRNAdb)29. The DNA templates (tDNAs) were synthesized by 3-step PCR (FIG. 8).

All DNA templates coding for peptide and sGFP were constructed based on pOPINE-eGFP plasmid (GenBank: EF372397.1). To construct peptide-coding DNA templates, two complementary oligonucleotides harboring NcoI and NotI restriction site overhangs were used to assemble the ORFs of the desired peptides. The concentration of oligonucleotides was adjusted to 100 μM, mixed in water at a 1:1 molar ratio followed by heating at 95° C. for 5 min, and then slowly cooled down to room temperature for annealing. The pOPINE-eGFP plasmid vectors were digested by NcoI and NotI, combined with the annealed oligonucleotides, and ligated using T4 DNA ligase. The positive clones were verified by Sanger sequencing (AGRF Brisbane).

The fragments coding for sGFP ORFs with various codon biases denoted as sGFP_T1, sGFP_T2, and sGFP_T3 were synthesized as G-blocks by IDT and cloned into the pOPINE-based plasmid following the standard Gibson cloning procedure.

### T7tRNA Synthesis and Purification

Standard run-off t7 transcription reactions were performed at 320 for 2 h in 40 mM Hepes-KOH (pH7.9), 18 mM Mg(OAc)2, 2 mM Spermidine, 40 mM DTT, 5 mM each rNTP containing 0.25 μM DNA template, 10 μg/ml T7 polymerase, and 0.25 U/ml yeast inorganic pyrophosphatase. For the synthesis of tRNAHis, the transcription reaction was first supplemented with 5 mM of each rATP/rCTP/rUTP and 6.8 mM rGMP for 5 min, followed by addition of 1.7 mM rGTP and incubation for 2 h. The DNA template for tRNAHis contained an additional G corresponding to −1 position in tRNA. After transcription, the reactions were diluted 5-fold into buffer C (125 mM NaOAc pH 5.2, 0.25 mM EDTA). The tRNA transcripts were purified by affinity chromatography using ethanolamine-Sepharose matrix. For 1 ml of transcription reaction, 0.2 ml of settled matrix was used. Following the 1-h incubation of the slurry at 40, the matrix with bound tRNAs was extensively washed with buffer C containing 200 mM NaOAc. t7tRNAs were eluted from the matrix into buffer C containing 2 M NaOAc. tRNA was ethanol precipitated and the pellets were dissolved in tRNA buffer containing 1 mM MgCl2 and 0.5 mM NaOAc (pH 5.0).

sGFP Expression by Semi-Synthetic tRNA Mixture

Three ORFs with variable synonymous codon compositions coding for sGFP were synthesized commercially and cloned into pOPINE plasmid. Template 1 (T1) had the highest codon variation, including five different synonymous codons coding for Leu, four for Val, and 3 three for Pro, Arg, Ser, and Thr (Table 4). Template 2 (T2) was designed to deliver the highest codon biases with only two synonymous codons used to encode Ser, Arg, and Leu, and one codon used to encode Val, Pro, Thr, Ala, and Gly (Table 5). Template 3 (T3) featured a medium codon variety with two codons for Ser and Arg, and several codons for Leu, Val, Pro, Thr, Ala, and Gly, as in T1 (Table 6). The proportions of individual tRNAs in the semi-synthetic tRNA mixtures were roughly proportional to their codon abundance in the sGFP ORF sequences, except for codons occurring more than 10 times and those corresponding to the least-depleted native tRNAs. These t7tRNAs in the semi-synthetic mixtures were taken at reduced proportions relative to their codon usage shown in Table 4-6. Production of sGFPs corresponding to T1-3 in the translation reactions with semi-synthetic tRNA complement was monitored on a fluorescence plate reader for 3 h at 485-nm excitation and 528-nm emission wavelengths.

### Results

To construct a synthetic E. coli tRNA complement capable of supporting protein translation, we conducted in vitro run-off transcription^{29,30} on 48 DNA templates harboring t7 promoter followed by the corresponding tRNA-coding sequences (FIG. 8).

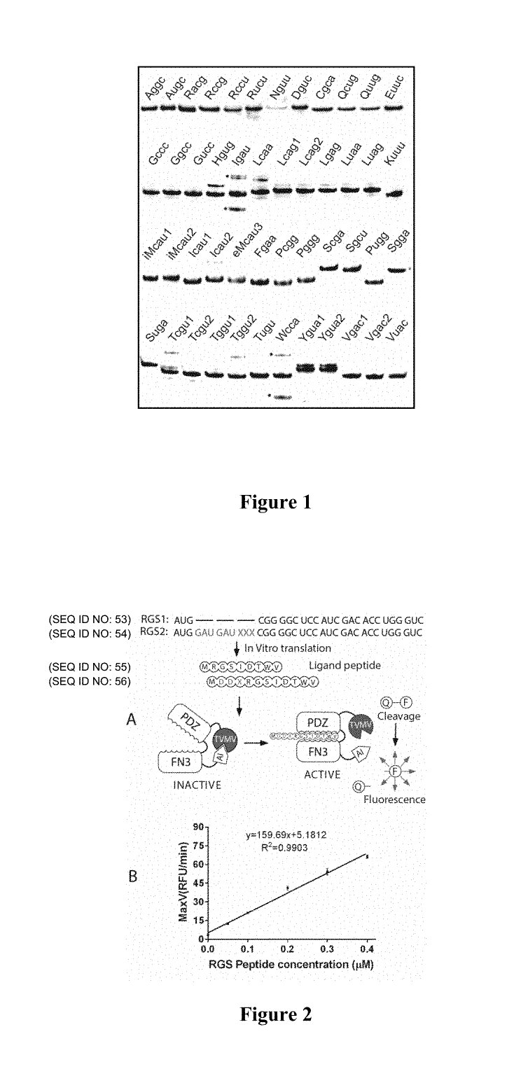

T7tRNA transcripts were obtained in good amounts for all tRNAs except tRNAlle(GAU) and tRNATrp(CCA) (FIG. 1). The sequences coding for these tRNAs start with adenosine, which is likely to cause high abortion rates in the early transcription phase.

For these two tRNAs, a Hammerhead ribozyme (HHRz) coding sequence prefaced by a strong transcription start site was introduced upstream to the tRNA coding sequences to ensure efficient transcription followed by HHRz-mediated auto-excision (FIG. 8)³¹. Denaturing PAGE analysis revealed that more than 90% of the RNA precursor was cleaved to yield the desired tRNAs (FIG. 1). Although some tRNAs, such as tRNALeu(CAA) and tRNATyr, contain additional minor bands, we obtained a major species of the expected size in all cases.

In Vitro Peptide Expression Assay Next, we sought to devise an in vitro synthesis assay to evaluate the decoding efficiency of t7tRNAs. We conjectured that a reporter peptide corresponding to a short open reading frame (ORF) that uses a limited set of codons is advantageous over classical reporter proteins such as GFP or luciferase that require a full set of tRNAs for their synthesis.
To establish a multiplexed quantitative homogeneous peptide expression assay, we took advantage of a peptide biosensor recently developed by our group³². It is composed of an artificially engineered peptide binding domain known as the “affinity clamp”³³ and an autoinhibited tobacco vein mottling virus (TVMV) protease. Binding of an 8-amino acid ligand peptide RGSIDTWV (SEQ ID NO:3) (RGS-peptide) to the biosensor triggers a conformational change resulting in protease activation, which is detected through cleavage of a quenched fluorescent TVMV substrate peptide (FIG. 2A). We demonstrated that as little as 50 nM of the peptide could be detected using this assay. The relationship between initial velocities of TVMV substrate cleavage (Vmax) and the absolute concentration of the RGS-peptide was found to be linear in the 50-400 nM range (FIG. 2B). Furthermore, the assay could be performed in an E. coli cell-free system, although this requires a cocktail of protease inhibitors to mitigate the endogenous proteolytic activity.

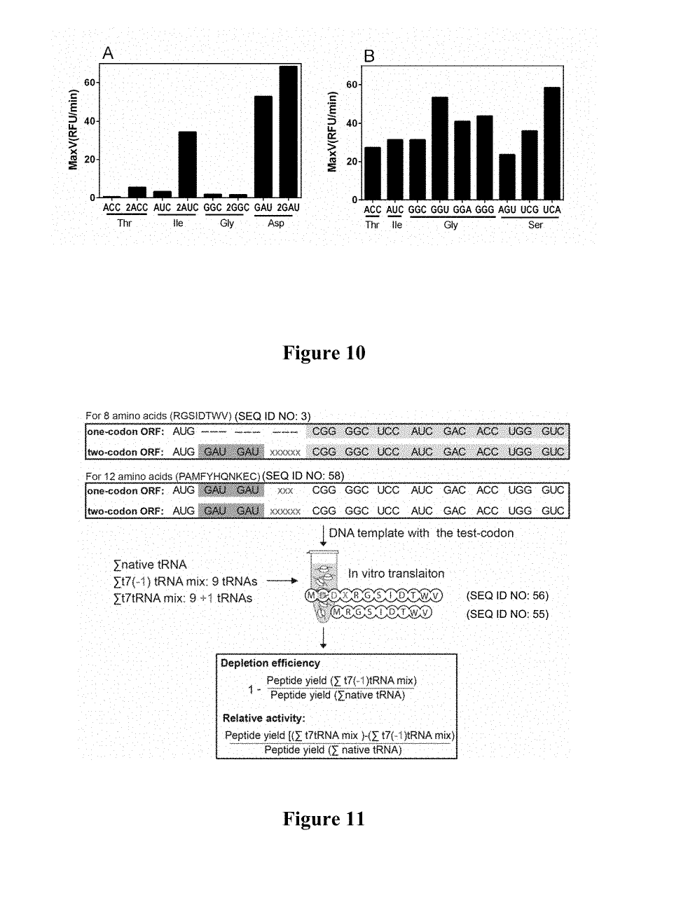

While binding of the RGS-peptide to the affinity clamp critically depends on a free C-terminus³⁴³⁵, introducing additional amino acids at the N-terminus of the RGS-peptide is not anticipated to affect the clamp-to-peptide binding³³. We confirmed this by testing two N-terminally extended synthetic peptides, GGRGSIDTWV (SEQ ID NO:4) and DDRGSIDTWV (SEQ ID NO:5), in our assay (FIG. 9), with the calibration curves for all three peptides displaying good linearity with R²>0.99 (FIG. 2B and FIG. 9). We concluded that our expression assay was well suited to quantify the expression of RGS-derived peptides in the cell-free system and thus could be used to test the decoding efficiency for various isoacceptor/codon pairs. To reduce the influence of codons immediately downstream of the initiator AUG codon on translation initiation, two GAU codons were inserted between the initiator and the test codons to yield an RGS2 template (FIG. 2A). Control experiments demonstrated that such a template mediated consistent peptide expression levels regardless of the test-codon upstream of the RGS-peptide coding sequence (FIG. 10).

Characterization of tRNA Depleted E. coli Cell-Free Translation System

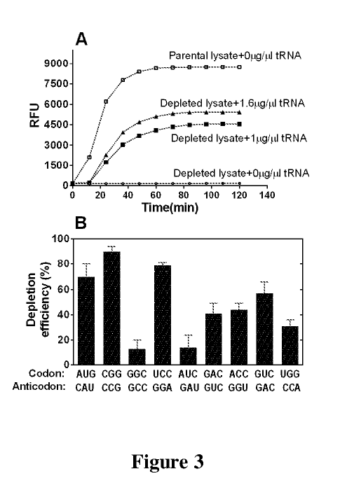

To obtain tRNA-depleted E. coli S30 cell extract, we modified a previously published chromatographic tRNA depletion protocol²⁸. In this procedure, the endogenous tRNAs bind to ethanolamine-Sepharose matrix while other components required for protein synthesis remain in the flow-through. To obtain optimal tRNA depletion while retaining the translation efficiency, we optimized the potassium/magnesium concentration as well as the matrix to lysate ratio. The extent of tRNA depletion was evaluated by comparing eGFP expressions in the depleted lysate with or without adding the total native tRNA mixture. As can be seen in FIG. 3A, no eGFP was produced in the absence of native tRNAs, while the addition of total native tRNA mixture restored translation to 60% of the parental lysate level. A time lag of approximately 10 minutes was observed in tRNA-depleted lysates, possibly reflecting the time required for aminoacylation of the re-added tRNAs.

Similar to eGFP, translation of a short RGS1 template (FIG. 2A) was also tRNA-dependent. Both native tRNAs and a mixture of 9 codon-specific t7tRNA species (Table 1) restored translation to similar levels (data not shown).

The observation that synthetic tRNAs could support translation of RGS1 template was somewhat surprising considering that tRNAIle requires post-transcriptional modifications for efficient aminoacylation²¹. Hence, we performed a control experiment where we omitted individual t7tRNAs from the mixture and measured the translational activities in the resultant reaction mixtures. As depicted in FIG. 3B, when the individual t7tRNAs for AUG(iMet), CGG(Arg), or UCC(Ser) codons were independently withheld from the t7tRNA mixture, peptide expression decreased significantly. However, when the t7tRNAs for GGC(Gly), AUC(Ile), GAC(Asp), ACC(Thr), or UGG(Trp) were excluded, some residual expression was observed, probably due to native tRNA remnants in the lysate. The depletion efficiencies of tRNAs for CGG(Arg) and UCC(Ser) were 75-90%, indicating that these codons are more suitable for reassignment due to negligible amounts of the respective native tRNA isoacceptors remaining in the depleted lysate (with the tRNA depletion efficiency defined as 100% when no peptide was produced without the addition of selected tRNA)³⁶. Both of these codons belong to mixed codon boxes composed of two codon families, making them particularly promising candidates for the reassignment (see below).

Systematic Analysis of t7tRNA Functionality and Specificity

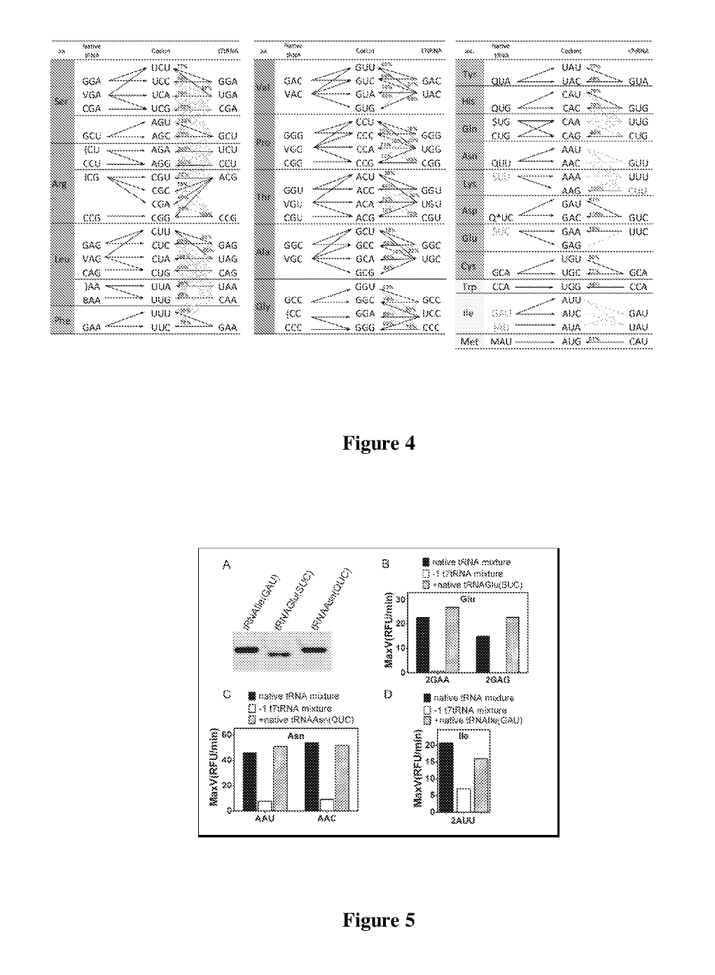

The developed assay provided a platform to systematically test the entire ensemble of t7tRNAs (FIG. 2A). Yet, the initial experiments revealed residual amounts of some isoacceptors in the depleted lysate. These represented tRNAs that are abundant in E. coli, indicating a relationship between the depletion efficiency of individual tRNAs and their abundance (Table 2)10,36. The incomplete depletion of endogenous tRNAs potentially complicates the functionality test by masking the signal from their t7tRNA counterparts. However, we observed that including two consecutive codons for a particular tRNA into the template significantly enhances the adverse effect of its depletion on the peptide translation efficiency. This is likely to reflect the changes in translation kinetics at reduced tRNA concentrations previously described for low-abundance tRNAs in vivo³⁷. Therefore, we rescreened codons with incompletely depleted isoacceptors using a reporter peptide ORF harboring two consecutive target codons. The decoding efficiency was calculated as the mean value of both one- and two-codon templates (FIG. 11). The ability of t7tRNAs to decode the 61 codons is summarized in FIG. 4 and FIGS. 12-14.

One important aspect to consider when interpreting the assay results is the extent to which t7tRNAs could undergo modifications in the lysate. For example, crude E. coli lysate was reported to mediate formation of pseudouridine in synthetic tRNAs³⁸. Such modifications require only isomerase activity, but not the low molecular weight substrates or cofactors potentially present in our system³⁹. However, the modifications on N34 and N37 involve up to 20 enzymes with relay chains, as well as multiple substrates and cofactors¹⁹. Therefore, such modifications are unlikely to emerge on the synthetic tRNAs in the crude lysate without significant optimization of the system. We experimentally address this issue later in the report (see below).

### Split Codon Family Boxes

Decoding of split codon families ending on U and C such as Ser, Phe, Tyr, His, Asn, Asp, and Cys is carried out by tRNAs with G or its modified form (Q) in the first anticodon position. The A- and G-ending codons are decoded either using modified uri uridine (Lys and Glu) or by adding isoacceptors with C in the first anticodon position (Arg, Leu, and Gln) (FIG. 4 blue and grey shaded amino acids). Uridine in the former case is modified with various aminomethyl derivatives that restrict the recognition solely to A- and G-ending codons⁴⁰, additionally supported by ribose 2′-O-methylation when U and C are in the first anticodon position of both Leu isoacceptors.

As compared to their native counterparts, t7tRNAs for Phe, His, Asp, and Cys were efficient (50-70%) in decoding both their cognate (C-ending) codon with Watson-Crick geometry (further referred to as “cognate-WC”) (C-ending) and wobble (U-ending) codons. The t7tRNATyr(GUA) decoded UAC with ˜50% efficiency and UAU with less than 30%. T7tRNAHis(GUG), featuring an additional G-1C73 base pair, demonstrated an effective decoding of its cognate-WC CAC and wobble CAU codons. The t7tRNA lacking G-1 was not functional in restoring peptide translation (data not shown), presumably due to failure of the aminoacylation step⁴¹.

The t7tRNASer(GCU) recognized both AGC and AGU codons with 123 and 75% efficiency, respectively. For Leu and Arg, two tRNA isoacceptors are responsible for decoding each split codon box, and in our assay, both Leu t7tRNAs with UAA and CAA anticodons recognized only their cognate codons via clasical WC-base pairing. This is in agreement with a previously reported restricted mode of recognition by unmodified uridine⁴², albeit with efficiencies of 36 and 88%, respectively. The t7tRNAs for Arg with UCU and CCU anticodons demonstrated similar behavior in strictly recognizing their cognate-WC codons at 340 and 202% efficiency, respectively. The higher apparent activity of these t7tRNAs possibly reflected the low abundance of these isoacceptors in the native tRNA mixture³⁶. Consistent with previous observations from the ribosome binding assay⁴³ t7tRNAGln(UUG) could not decode its cognate-WC CAA codons. The t7tRNAGln(CUG) also could not decode CAA codons, although it could decode its cognate-WC CUG codon with 40% efficiency.

T7tRNAs for Glu(UUC), Ile(GAU), Asn(GUU), and Lys(UUU) failed to sustain peptide translation from the template comprising both their cognate-WC and wobble codons. Lack of modifications within the anticodon loops of Glu and Ile t7tRNAs was previously shown to prevent their aminoacylation, making them inactive in the peptide translation^{44,45}, and t7tRNAAsn(GUU) prepared with or without the help of Hrz performed poorly in the reporter peptide synthesis. Although chimeric t7tRNALys, with the grafted anticodon and the discriminator base both derived from tRNAAsn, could be aminoacylated by AsnRS with Asn⁴⁶, it still failed to support peptide expression in our assay (data not shown). In this regard, it was previously reported that tRNALys, with unmodified U34, failed to decode either of its codons due to the potential loss of structural order in the anticodon loop as well as poor stacking within the codon-anticodon duplex formed by three consecutive, least-overlapping A-U base-planes^{47,48}. In our system, mutating U to C in the first anticodon position of t7tRNALys fully restored its decoding activity towards AAG-codon. This effect can potentially stem from the stronger stacking provided by cytidine within both the anticodon loop and the codon-anticodon helix, as well as from a higher affinity towards lysyl aaRS⁴⁹.

T7tRNAs for Trp(CCA) and Met(CAU) decoded their cognate-WC codons with 46% and 61% efficiency, respectively.

### Unsplit Codon Family Boxes

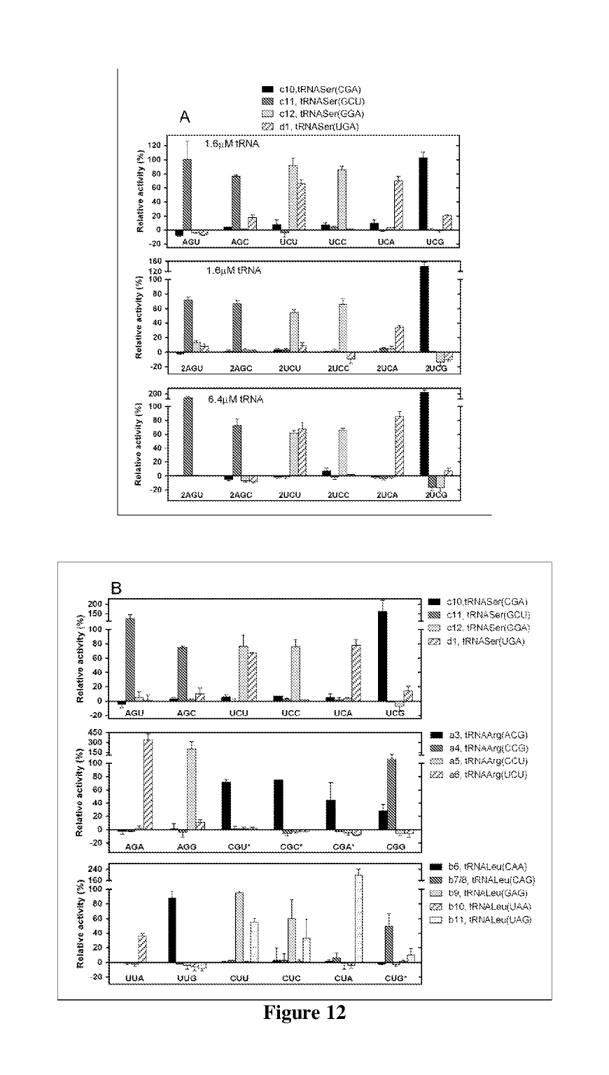

With the only exception of Arg, the standard subset of 2 or 3 isoacceptors bearing G, U, and/or C in the first anticodon is employed in bacteria to decode 8 unsplit codon family boxes. Here, G34 pairs with C- and U- and C34 to exclusively mediate the decoding of G-ending synonymous codons (FIG. 4 blue and green shadings). In the peptide synthesis assay, all t7tRNA isoacceptors with G or C in the first anticodon position demonstrated specific recognition of their cognate-WC codons with efficiencies of 40 to 160% (FIG. 4). In all native tRNA isoacceptors except tRNAGly(UCC), U34 carries a 5′-oxyacetic acid modification which extends recognition beyond its cognate-WC A-ending⁵⁰ to G-, U-, and C-ending codons for Val, Pro, and Ala by partially altering the nucleoside sugar pucker geometry^{51,52}. Furthermore, t7tRNALeu(UAG) and t7tRNASer(UGA), which are presumably devoid of modifications, show strong preferences for A- and to a lower degree U-, but fail to recognize G- and C-ending codons²⁴. The relative efficiency for tRNASer(UGA) decoding two consecutive UCU codons at 1.6 μM was ˜10%, while an increase in the isoacceptor concentration to 6.4 μM resulted in ˜70% decoding efficiency (FIG. 12A). This finding can be easily rationalized considering the lack of some post-transcriptional modifications in the tRNA body (including the anticodon loop) leads to either a reduction in affinity towards aaRS or a higher rate of dissociation of the codon-anticodon interaction and/or tRNA accommodation⁵³. Both of these effects could, at least partially, be compensated by the increase in tRNA concentration.

Surprisingly, t7tRNAs for Val, Pro, Thr, and Ala with most likely unmodified U34) displayed a similar codon-reading pattern to their native counterparts—i.e., these t7tRNAs not only efficiently decoded their cognate-WC A-ending codons, but also to a lower degree the U- and C-ending ones⁵⁴. Decoding of G-ending codons features strong U34-G3-mediated recognition for Val and Ala, which lack C34-bearing back-up isoacceptors. This contrasts the inefficient U34-G3-mediated recognition for Pro and Thr (FIG. 4) that is possibly mediated by the cognate-WC isoacceptors.

Native tRNAGly(UCC) differs from other tRNAs decoding unsplit boxes with U in the first anticodon position, in that it carries aminomethyl modifications at U34 (see above), which is characteristic of tRNAs decoding split codon boxes⁵⁰. T7tRNAGly(UCC) is an exception to the above-described correlation as it effectively decodes C- and G-ending codons despite the existence of C34 isoacceptor for the cognate-WC decoding of the latter.

As mentioned above, decoding of four Arg codons from the unsplit family box in bacteria is unusual because it relies on two isoacceptors, one of which carries an inosine modification more common in eukaryotes. This modification enables decoding of A-, U-, and C-ending codons via base pairing with wobble and WC-geometries, respectively. The unmodified anticodon stem-loop of t7tRNA(ACG) showed almost the same affinity to its cognate-WC codon CGU, but was inefficient in binding to its wobble CGC and CGA codons⁵⁶. In our study, t7tRNAArg(ACG) could efficiently decode not only U-, but also C-, A-, and, to a lower extent, G-ending codons.

The experiments described thus far demonstrate that the synthetic tRNAs could functionally replace their native counterparts in vitro for 17 amino acids. Importantly, for all tested t7tRNAs, no cross-recognition was observed either for codons from the same family coding different amino acids (FIG. 4, Ser and Arg, Leu and Phe, His and Gln, Asn and Lys) or for synonymous codons belonging to different families with the same mixed codon boxes such as Ser, Arg, and Leu. Analyzing these three amino acids with split codon families joined with unsplit codon boxes is particularly interesting because the former and the latter possess non-overlapping decoding patterns and represent potentially reassignable codons.

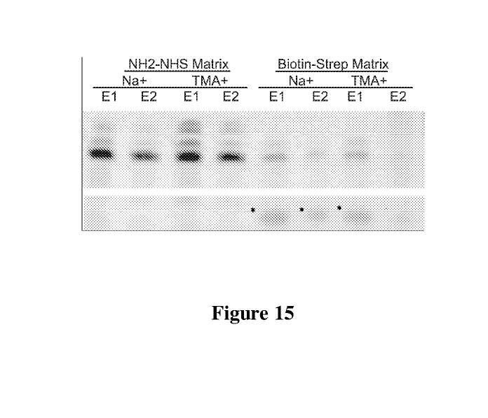

Isolation of native tRNAs for Glu, Asn, and Ile from the E. coli native tRNA mixture Our results demonstrated that the majority of amino acids could be incorporated into protein by using in vitro-transcribed tRNAs. To reconstitute a tRNA mixture capable of supporting translation of proteins containing all 20 canonical amino acids, we needed to efficiently decode codons for Glu, Asn, and Ile. To this end, we decided to purify native tRNAs specific for these amino acids from the native tRNA mixture by DNA/RNA hybridization chromatography^{57,58}. We tested several immobilization strategies and obtained the best results by coupling 3′-aminated oligonucleotides to NHS-sepharose (FIG. 15). All three specific tRNAs for Glu, Asn and Ile were successfully obtained at good purity from the native tRNA mixture by selective hybridization with oligonucleotides complementary to the D-loop and the anticodon loop of the target tRNA (Table 3). The tRNAs were eluted from the matrix by thermal denaturation and were shown to be of >90% purity by denaturing PAGE analysis (FIG. 5A). The functionality of purified native tRNAs for Glu(SUC), Asn(QUU), and Ile(GAU), was tested as described above using templates harboring their cognate-WC or wobble codons.

Two templates with consecutive GAA or GAG codons were employed to test the functionality of purified tRNAGlu(SUC), which, as shown in FIG. 5B, could efficiently decode both codons. Similarly, the purified native tRNAAsn(QUC) restored the translation of templates harboring either AAU or AAC codons. The purified native tRNAIle(GAU) could only decode AUU codons with 40% efficiency, possibly due to inefficient refolding after denaturation or co-isolation of the under-modified isoacceptor variant.

### Semi-Synthetic Protein Translation System

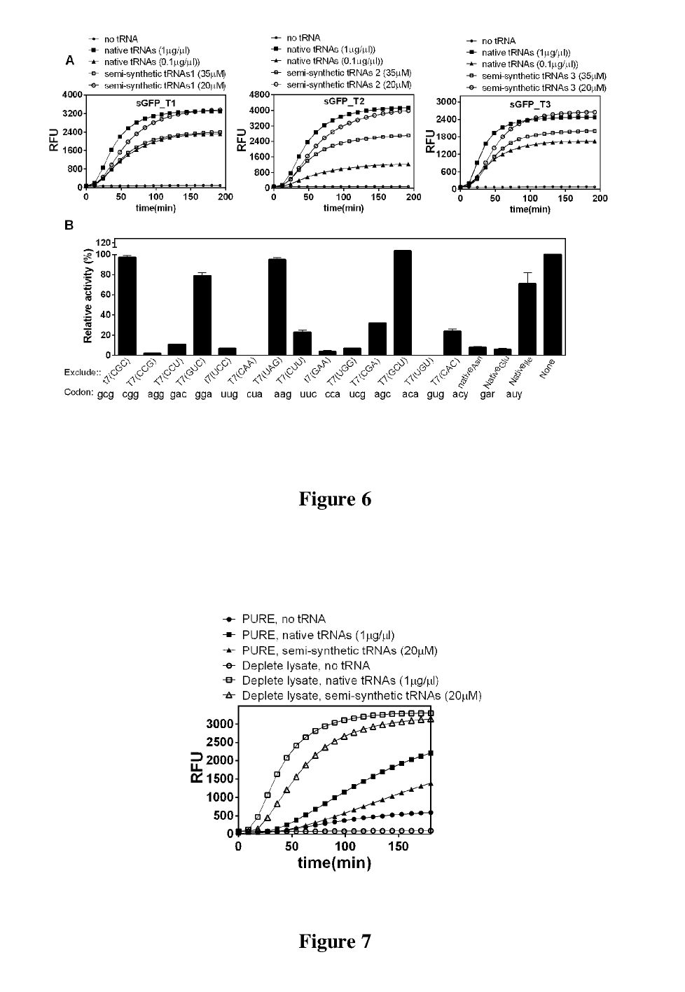

We obtained at least one purified functional tRNA for each of the 20 canonical amino acids. To test whether this simplified tRNA complement could support synthesis of a full-length protein, we synthesized three DNA templates encoding for superfolder GFP(sGFP)59 with variable codon compositions. These templates were designed to exclude codons inefficiently decoded by synthetic tRNAs such as CCU(Pro), UAU(Tyr), CAA(Gln), and AAA(Lys) (Table 4-6). The templates were expressed in tRNA-depleted lysate supplemented with different semi-synthetic tRNA mixtures (Tables 4-6), which supported translation of all three templates with efficiencies comparable to the native tRNA mixture (FIG. 6A).

To ensure the sGFP expression was a direct result of supplementation with semi-synthetic tRNAs and to reconfirm the functionality of the individual tRNAs, we formulated tRNA mixtures lacking individual tRNAs. We then analyzed the ability of these mixtures to support synthesis of sGFP templates in a tRNA-depleted cell-free system and observed a reduction in translation ranging from several-fold to orders of magnitude (FIG. 6B). Similar to the results obtained by peptide expression assay, including the t7tRNAs coding for UCG(Ser), CGG and AGG(Arg), UUG(Leu), GGA(Gly), CCA(Pro), ACA(Thr), GUG(Val), AAG(Lys), and UUC(Phe), the semi-synthetic tRNA mixtures restored sGFP expression, thus reconfirming the functionality of the corresponding t7tRNAs. However, in contrast to the RGS-peptide expression profile, residual amounts of native tRNAs for CUA and AGC codons proved to be sufficient for sGFP expression. When only one CUA or AGC codon was present in ORF sequence of the reporter peptide, the presence of t7tRNA(UAG) and t7tRNA(GCU) in the mixture restored peptide expression to 92 and 72%, respectively (Table 2). This inconsistency possibly reflected a higher concentration of peptide transcripts as well as higher turnover rates of peptide translation compared to sGFP. In the former case, the number of elongating complexes were possibly surpassing the number of native tRNAs remaining in the lysate, while in the latter case the same tRNA could be tunneled within the same polysomal unit 37,60 When just one codon was present in the ORF, sGFP expression appeared to be more sensitive to the residual amounts of corresponding native tRNAs in the depleted lysate.

Unlike for the CUA and AGC codons, the expression level of sGFP with only one AGG codon per ORF decreased by ˜90% when t7tRNA for AGG was excluded. This makes AGG the most promising codon for reassignment even without further optimising the depletion of native tRNAs.

The observation that full-length protein could be ex-pressed in our cell-free system prompted us to probe the role of tRNA modifications in protein translation. As discussed above, even though the majority of tRNAs in our system were synthetic, they could potentially undergo partial editing or modifications in the context of a translationally active lysate.

To test the effect of such putative modifications on the functionality of t7tRNAs, we repeated the above experiment using the reconstituted PURE in vitro translation system, which presumably lacks tRNA processing and modification activities. We found that semi-synthetic tRNAs sustain protein expression—yet, we observed that the relative translation efficiency of the PURE system supplemented with semi-synthetic tRNAs was only 60% compared to the native tRNA complement. This is in contrast to our observation that both native and synthetic tRNA complements performed almost equally well in depleted lysates. On the one hand, this indicates that unmodified synthetic t7tRNAs could sustain protein synthesis. On the other hand, the observed reduction in efficiency may reflect additional post-transcriptional processing of at least some t7tRNAs in the S30 extract, but not in the PURE system. Addressing this issue conclusively would require testing the functionality of individual t7tRNAs in the PURE system. This is not straightforward due to the surprisingly high levels of contaminating native tRNAs (FIG. 7).

### Discussion

In this work, we established an approach for the systematic analysis of individual tRNA functions using an in vitro translation system depleted of endogenous tRNAs. This was achieved by developing a non-radioactive assay to quantify the in vitro expression of a reporter peptide in tRNA-depleted E. coli cell-free translation systems. We demonstrated that the depleted lysate retained more than 60% of the activity of the parental lysate. Although the residual tRNA pool in depleted lysate could not sustain eGFP and RGS-peptide expression, we found that a subset of native tRNAs was not fully depleted. The developed peptide biosensor assay allowed us to estimate the depletion level of tRNA isoacceptors relative to their codons and revealed a correlation between the depletion efficiency of individual tRNAs and their abundance. Importantly, RGS1-peptide expression was also observed in a fully recombinant E. coli PURE system primed with t7tRNA mixtures lacking individual tRNAs (data not shown). Furthermore, the PURE system assembled without exogeneous tRNAs could support the expression of full-length GFP (FIG. 7), indicating presence of the entire spectrum of contaminating tRNAs. This suggests that residual tRNAs most likely copurify with aaRSs or other components of the translational machinery 16. Therefore, efficient tRNA depletion from in vitro translation systems remains a challenge.

To distinguish the activity of t7tRNAs from the endogenous tRNA background activity, we utilized the observation that two identical, consecutive test-codons significantly sensitized the assay to the depletion of specific tRNAs. This enabled us to analyze the functionality of synthetic versions of all 48 E. coli tRNA species in our tRNA-depleted lysate.

Our results demonstrate that most of the synthetic tRNAs were efficient in supporting protein/peptide translation (FIGS. 4-7). Furthermore, most of the t7tRNAs corresponding to 2- or 4-fold-degenerate amino acids decoded their cognate-WC codons with high or medium efficiency and their wobble codons with medium or low efficiency (FIG. 4). In contrast, the t7tRNAs for Asn, Gln(CAA), Ile(AUC), Glu, and Lys were found to be non-functional in the affinity clamp assay.

Even though we could not exclude the partial editing and modification of synthetic tRNAs in the crude translation system, it appears unlikely that the N34 and N37 modifications, which require the activity of multiple enzymes, would occur efficiently. This notion is supported by the observation that synthesis of Ile, Glu, and Lys could not be supported by synthetic tRNAs, and accords with previous studies showing that modified nucleotides served as key molecular recognition features for their cognate aaRSs²¹. It was reported earlier that tRNALys(UUU) lacking modifications outside the anticodon loop undergoes aminoacylation at 140-fold lower efficiency⁶², yet, conversion of U to C in the first anticodon position restored its activity towards the AAG codon. In addition to Lys(UUU), a number of t7tRNA transcripts or their corresponding anticodon stem-loops such as Arg(UCU), Ala(UGC), Cys(GCA), Glu(UUC), and Gln(UUG) have also failed in the ribosome-mediated codon binding assay⁵⁰. With the exception of Glu(UUC) and Gln(UUG), three remaining t7tRNAs were translationally active in the affinity clamp assay. In particular, t7tRNAArg(UCU) demonstrated a 3-fold higher activity compared to its homolog, which is presumably underrepresented in total native tRNA mixtures (FIG. 4). Overall, the codon-anticodon interaction matrix depicted in FIG. 4 shows highly similar codon recognition patterns between native and t7tRNAs, which are most likely devoid of modifications within anticodon loops. For instance, U34 in the first anticodon position of t7tRNAs decodes not only its cognate A and G, but also U and C in the third codon position with similar reading patterns to that of cmo5U in the native tRNAs for Ser, Leu, and Gly, and with an identical pattern in tRNAs for Val, Pro, Thr, and Ala. From the structural and kinetic data, it appears that the intrinsic stability of the codon-anticodon helix is less important than its proper geometry, which is sensed by the ribosome^{63,56,57}. These studies thus imply that the net affinity between the codon-anticodon duplex and the ribosome promotes 30S closing around the decoding center, thereby promoting tRNA accommodation and triggering the downstream steps that lead to peptide-bond formation⁵⁴. In the current study, the highly mosaic pattern of U34-N3 interactions and the lack of non-cognate cross-recognition in the split codon boxes supports the idea of higher order contextuality in the tRNA body—potentially, an additional check-point for accurate and productive decoding^{56,57}.

The decoding preferences shown here provide a valuable guide for identifying “orthogonal” vs. “native” codon pairs from the synonymous codons for a particular amino acid. Such pairs can either be created from the codons of different families of 6-fold-degenerate amino acids or from those derived from the unsplit codon family boxes with high wobble restrictions such as Arg, Ser, and Leu and Pro, Thr, and Gly (Table 7). This work suggests that the AGG-codon, for which native tRNA was depleted almost completely, is potentially easier to reassign than all the other codons.

We showed here that all amino acids except Ile, Glu, and Asn could be decoded by synthetic tRNAs, and that native tRNAs for these three amino acids were purified to homogeneity in a functional form. We demonstrated that the tRNA complement reconstituted with synthetic tRNAs and three specific native tRNAs could support in vitro synthesis of sGFP to comparable levels achieved with the native tRNA mixture. Although the full tRNA depletion remains a challenge, our results using the PURE system provide a clue to the origin of the contaminating tRNA pool.

Improved tRNA depletion protocols in combination with semi-synthetic tRNA complements would enable reassigning sense codons in peptides and proteins, thereby significantly expanding the toolbox of synthetic biologists and protein engineers. Further, the developed peptide expression assay in combination with the PURE system enables the impact of individual tRNA modifications on their functionality to be dissected. This should in turn answer a long standing question regarding the extent to which such modifications need to be maintained in the effort to construct the minimal cell⁶⁴. Finally, the presented approach is not confined to E. coli, and can be transferred onto eukaryotic cell-free expression systems.

### Additional Supporting Information

Preparation of 48 t7tRNA Transcripts

We used 3-step PCR to prepare two types of DNA templates for tRNA in vitro transcription. First type was assembled by 2 forward (T7, F) and 3 reverse oligonucleotides (R1, R2, R3) for tRNA species starting with G, U and C (FIG. 8A) while the other type was assembled from 3 forward (T7, F1, F2) and 3 reverse oligonucleotides for tRNA species starting with A and U to generate a self-processing Hammerhead ribozyme (HHRz) fusion constructs (Fig S1B)². F1 primer contained the T7 promoter followed by GGGAGA sequence to reduce the transcription abortion rate, 4-10 bases complementary to 5′-part of target tRNA and a fragment of HHRz-coding sequence. F2 contains HHRz-coding sequence followed by a segment complementary to 5′-part of tRNA. Following a 3-step PCR, the products were purified by ethanol precipitation and employed as templates for T7-transcription as described in Materials and Methods in order to obtain the desired tRNAs.

Identity of 3′ and 5′ of t7tRNA Transcripts

The generated synthetic tRNAs are expected to contain physiological 3′-hydroxyl group. Depending on the preparation method the 5′- can bear 5′-monophosphate, 5′-triphosphate, or 5′-hydroxyl groups. The non-physiological 5′-triphosphate may be accepted by aaRS and other translational machinery^{67,68} or can be processed to monophosphate in the crude lysate by the endogenous phosphatase activities, such as for instance of RNA pyrophosphohydrolase⁶⁹. In the cases where 5′-ribozyme-mediated processing of the transcript was employed, the cleaved products contain 5′-hydroxyl which has been reported not to affect the aminoacylation efficiency for tRNA transcripts of Ser, Met, Phe, Tyr, Asp⁶⁶. Although 5 of the 48 tRNA genes harbouring “CAT” anticodon are assigned to Met in Genomic tRNA database⁶, only 3 of them actually code for Met (2 for initiator and one for elongator Met codons) while the remaining 2 code for Ile according to the sequences from Modomics database⁷¹.

### Optimisation of the RGS Templates for Efficient Peptide Expression

To test the functionality of t7tRNA species for the codons not present in RGS-peptide sequence, the test-codons were initially inserted between the RGS-peptide sequence and the initiation codon. However, the inserted codons in the vicinity of initiator AUG could influence the translation initiation⁷² thereby complicating the interpretation of the results. Hence we decided to insert one or two invariable “insulator” codons following the start codon to minimise the effect of the downstream codon insertions. For this purpose, we tested four different codons of varying G/C content such as ACC for Thr, AUC for Ile, GGC for Gly, GAU for Asp and placed one or two of these codons between the AUG start codon and the peptide-coding frame. Among them, the template harbouring two consecutive GAU codons showed the best translation efficiency as evidenced by the affinity clamp assay while there were low or no signal from other templates (FIG. 10A). We conjectured that the peptide-terminal Asp facilitated the peptide release from the ribosome exit tunnel by conferring a net-negative charge to the former⁷³. We constructed several DNA templates with variable codons following the two invariable GAU codons that all performed well in the affinity clamp assay regardless of the codons used (FIG. 10B). Therefore, we used this parental template for testing all other codons.

Optimization of Native tRNA Purification Strategy

### Preparation of the Immobilized Oligonucleotide Matrix

The commonly used strategies for immobilization of oligoDNAs are based on biotin-streptavidin interactions⁷⁴. However this robust and flexible approach was suboptimal for our purposes due to matrix instability under the denaturing conditions used for tRNA elution. Therefore, we tested immobilization chemistries based on amine-NHS and thiol-iodoacetamide conjugation.

OligoDNAs with 3′-amine, 3′-biotin or 3′-thiol groups were synthesized by IDT. OligoDNAs with 3′-thiol required reduction before immobilization, which added the complexity to the immobilisation protocol. OligoDNAs functionalized with amine, biotin and thiol at 112, 150 and 55 nmole respectively were immobilized to yield 1 ml of the respective settled resins. Due to high cost, complicated protocol and low conjugation efficiency the immobilization via thiol-reaction with iodoacetyl-resin was abandoned. Since the N-hydroxysuccinimide sub-product of amine/NHS conjugation reaction had high absorbance at 260 nm and strongly influenced the quantification of unbound OligoDNA, the ratio of bound vs unbound OligoDNA as a measure of conjugation extent was determined by non-denaturing PAGE of the reaction supernatants.

tRNA Purification

It was reported that tetramethylammonium chloride (TMA-Cl) could enhance the formation of tRNA-oligoDNA hybrids⁷⁵. Therefore, besides the standard hybridization buffer containing 0.9M NaCl we also tested a buffer containing 0.9M TMA-C1. From 100 μg of total native tRNA mixture, around 2.25 μg of tRNAGlu was purified on the amine-conjugated matrix which corresponded to 1 nmole of immobilized OligoDNA while the yield from streptavidin matrix with oligoDNA immobilized through biotin was approximately 3-fold lower. In the latter case the eluted tRNA contained a fraction of co-eluted at elevated temperature OligoDNA migrating on the gel below the front of tRNA (marked with asterisk in FIG. 15). Therefore, NHS-matrix was used for purifying the desired subset of native tRNAs. The final purification protocol based on the reported method¹¹ was further improved by optimizing the washing steps as described in the corresponding section of Materials and Methods. All native tRNAs from the required subset except tRNAAsp were obtained at a good yield and high purity (FIG. 6).

### Example 2

### Selective Depletion of tRNAs Using Kissing Loops

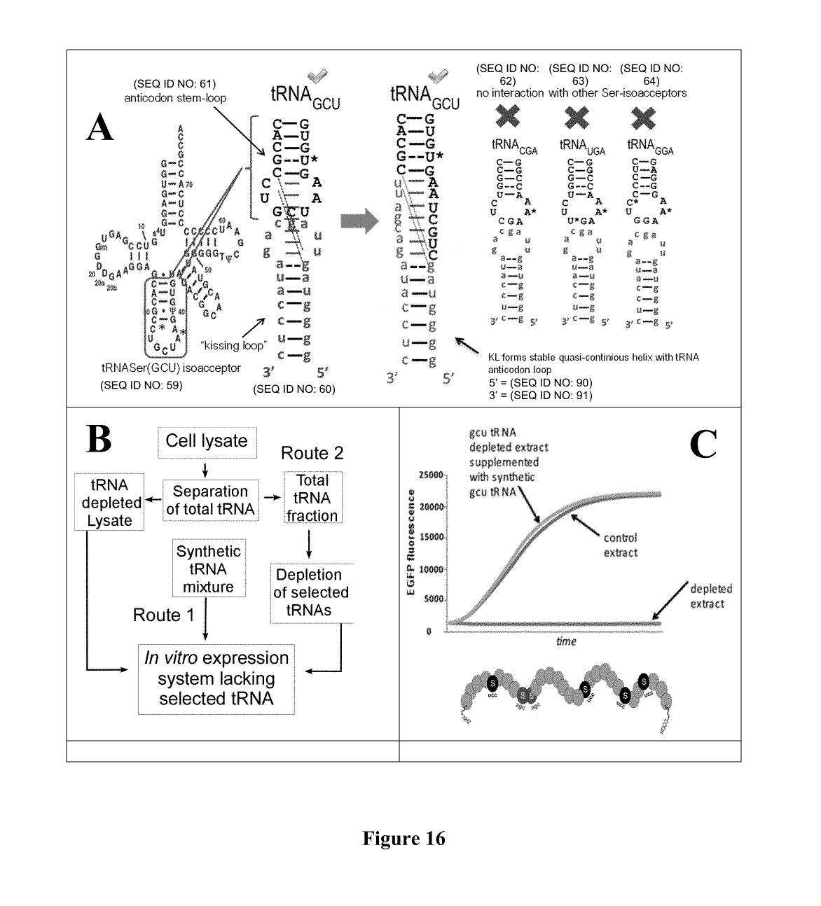

Kissing loop (KL) binds GCU Ser isoacceptor with high affinity (5-50 nM) through the formation of quasi-continuous double helix stabilized by the common stacking column between the planes of the nucleotide pairs (FIG. 16A). E. coli S30 lysate can be to a large extent depleted of total tRNA by the chromatography on ethanolamine sepharose. After the depletion lysate retains ˜60% of translational efficiency of its parental one if supplemented by commercially available total tRNA fraction (FIG. 16 B).

KL-immobilized matrix can be used either for specific tRNA-isoacceptor depletion directly from the crude lysate or indirectly by pulling the isoacceptor out of the total tRNA mixture followed by its reconstitution with all-tRNA depleted lysate (Fig (B)). GFP-synthesis in the reaction prepared using either method demonstrates ˜100% pausing on the codon-biased template with two-consecutive AGC-codons (FIG. 16 C). For this purpose the “kissing loop” coding sequence (upper case) GGTAGTGAGGTAGTTAGCAGATACCTCACTACCaacacacacacaacacacacacaacacacaagct (SEQ ID NO:7) with the linker (lower case) was cloned downstream the T7 promoter 3′-flanked by the HindIII site, followed by runoff T7-transcription from the HindIII digested plasmid. In order to obtain the KL-immobilized matrix, the KL-RNA was oxidized by sodium periodide and conjugated with the adipic acid dihydrazide-agarose beads (Sigma: A0802-50ML) accordingly to the PMID: 14652075. In the first approach two batches of 1 g of matrix containing ˜20-25 nmol of KL were incubated repetitively with 2.5 ml of crude lysate buffered with 5 mM Mg(OAc)2, 25 mM HepesKOH (pH7.6), 0.1 mM EDTA 30 min at RT, followed by buffer exchange on P-10 gel-filtration columns. In the second approach 125 mg of KL-beads were repetitively incubated in 200 μl reaction volume containing 5 μl of 30 mg/ml total E. coli tRNA (Roche: 10109541001) at the same conditions, followed by precipitation of tRNA by ethanol.

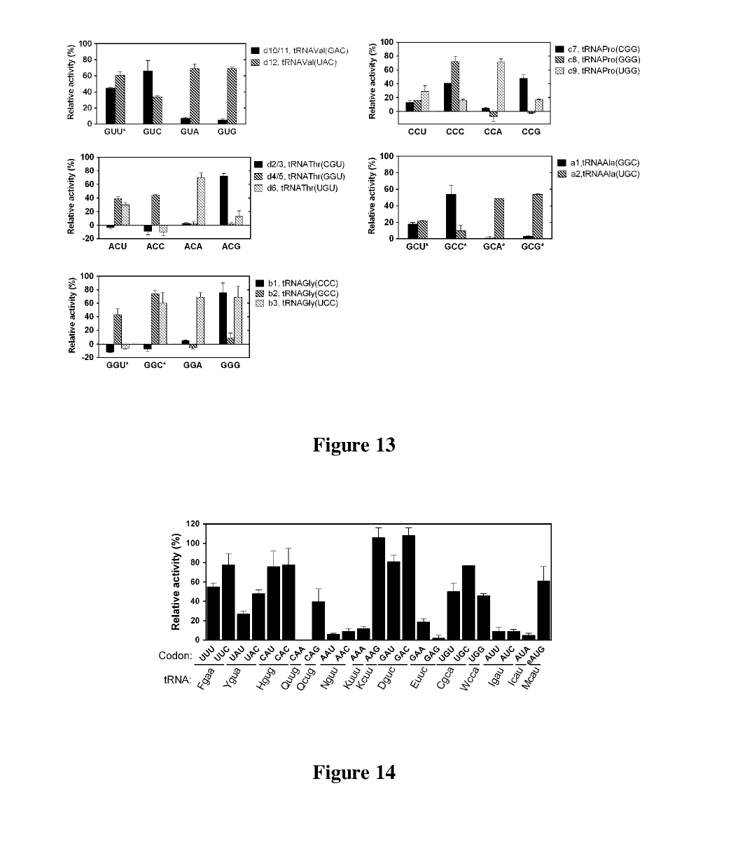

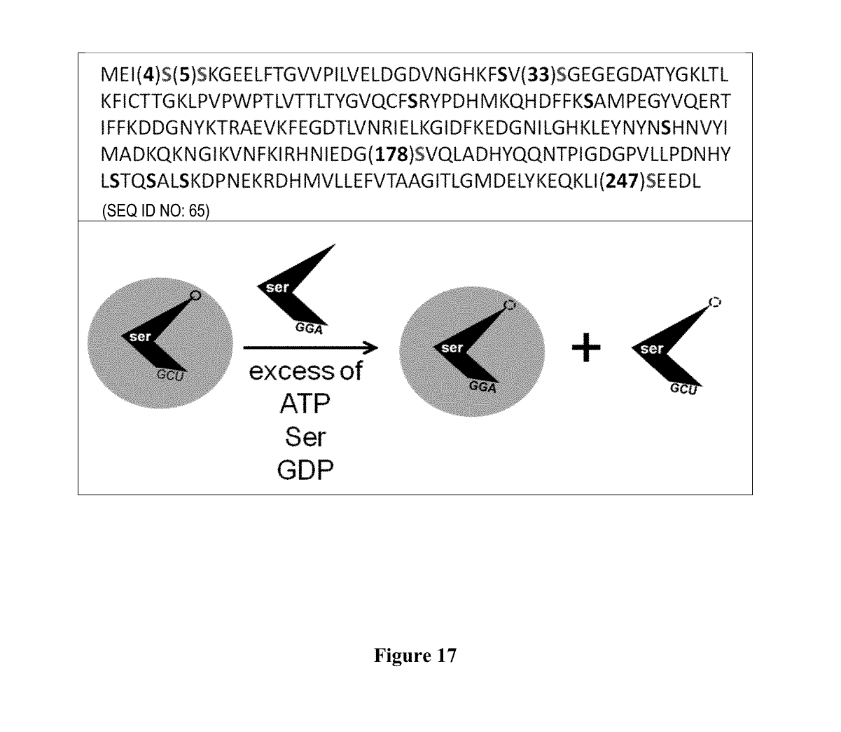

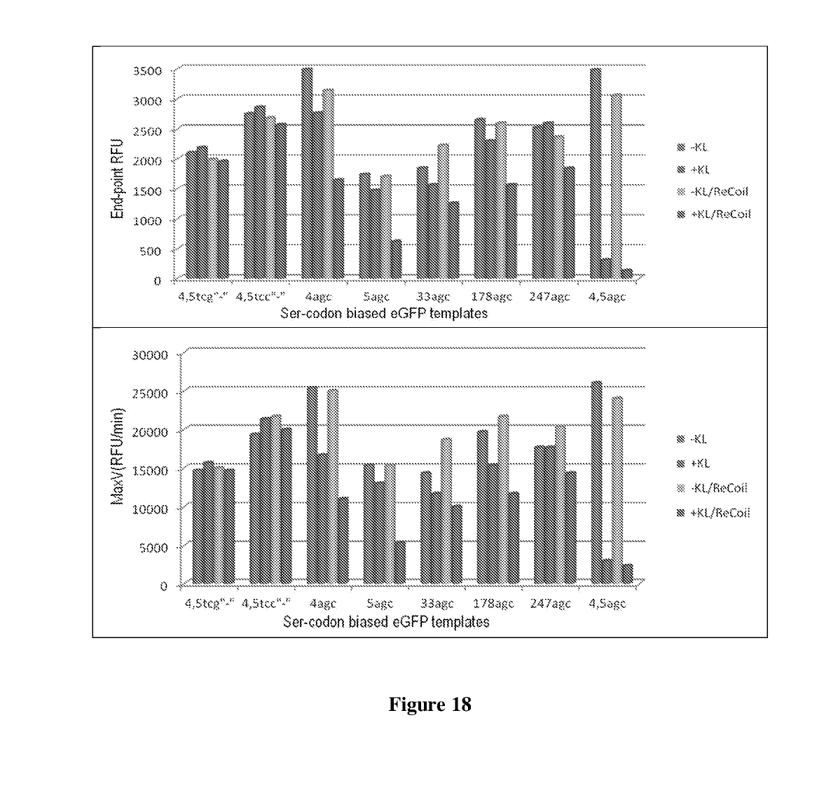

It was found that tRNA(Ser)GCU isoacceptor depletion is high, although incomplete—some small tRNA fraction seems to be a part of complexes with either aminoacyl-tRNA synthetases (ARSases) or EF-Tu thus escaping the KL-trap. We attempted to achieve better depletion by undertaking “ARSase recoiling” procedure (see FIG. 17, lower panel). In this approach, translation lysate is incubated with the KL-immobilized resin in the reaction mixture described above but in addition containing 23 mM of Mg(OAc)₂, 5 mM of each KOAc and KCl in the presence of a) excess of counter-isoacceptor (tRNA(Ser)gga) (15 μM) which is responsible for the decoding of all Ser-codons in codon-biased template except target-AGC (decoded by the isoacceptor being depleted) b) excess of other two substrates of the seryl-aminoacyl-tRNA synthetise (SerRS) such as ATP (20 mM) and serine (5 mM) as well as c) 1 mM GDP in order to dissociate the complexes of Aa-tRNA with EF-Tu. Excess of SerRS substrates accelerates the turnover kinetics of the enzyme leading to an effective isoacceptor replacement where the isoacceptor of interest tRNA(Ser)GCU is released and becomes available for KL-trap. While translating Ser-codon biased templates (FIG. 18, upper panel) comprising either no AGC-target codons (4,5tcg“-”; 4,5tcc“-”), 2-consecutive AGC-codons (4,5agc) or single target-codons at different positions (all the rest) lysates undergone “ARSase recoiling” procedure (FIG. 13, 3^{rd} and 4^{th} panels: green and purple bars) display higher tRNA depletion extent as compared to controls for which no “recoiling” was done (FIG. 13, blue and red bars).

### Example 3

### In Vitro Aminoacylation

In order to carry out site-selective incorporation of uAAs, we initially established three protocols for in vitro tRNA aminoacylation. The first is based on a protocol for CysRS-mediated in vitro aminoacylation of mutant tRNA(Cys). The reactive thiol group enables rapid derivatisation of acylated tRNA(Cys) with a spectrum of maleimide- or iodoacetamide-activated reagents. Incorporation of BODIPY-Cys was obtained through this route into a unique AGG codon of EGFP. We have also established pyrrolysine RS (PylRS)-mediated acylation of pyrrolysine cuAtRNA and Flexizyme-mediated tRNA aminoacylation. We demonstrated that we could incorporate Lys e-amino that has been derivatised by trans-cyclooctene (Lys-COC) into the reassigned Ser AGC codon in an E. coli cell-free system. Importantly, we observed that incorporation of COC into a sense codon has a much smaller impact on protein expression yield compared with amber suppression.

### Example 4

### Cysteinyl tRNA Synthase-Mediated Aminoacylation

Cysteinyl-tRNA synthetase (CysRS) has a contact area with the anticodon triplet of tRNA(Cys). Although the affinity of the enzyme towards the mutated tRNA is reduced it still can support high aminoacylation rates at high substrate concentrations in vitro however, we found that such mutant tRNAs were unable to undergo efficient re-acylation in the course of translation reaction due to an insufficient concentration of endogenous CysRS as well as high competing activity of wild type tRNA(Cys). This ensures that only a uAA is incorporated into the growing polypeptide chain.

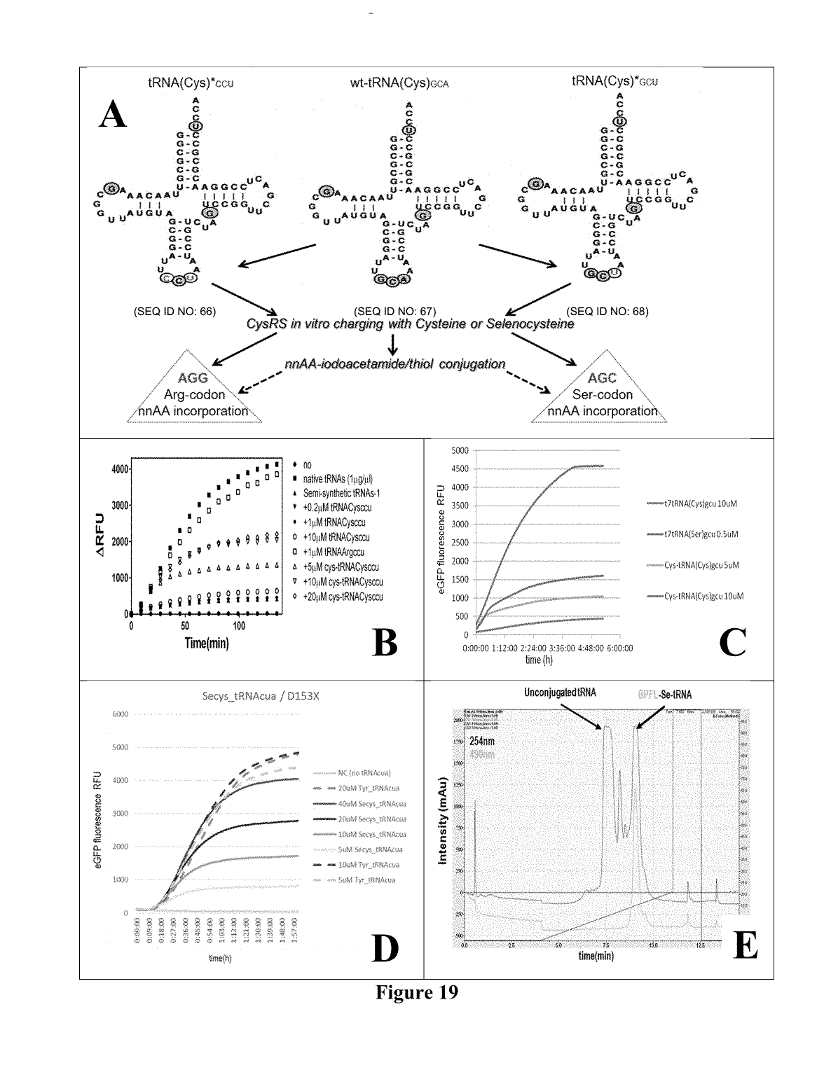

The anticodon of wt t7tRNACys GCA was changed to either CCU or GCU to match the corresponding Arg-AGG or Ser-AGC codons (see FIG. 19A). Amber-codon suppressor t7tRNACys with CUA anticodon was used to optimize the aminoacylation conditions by monitoring the fluorescence yield in the translation reaction primed with eGFP-ORF where aspartate codon at position 153 was converted to amber-stop codon. Both t7tRNAs were charged with cysteine by recombinant CysRS in vitro in the reaction containing 50 mM HepesKOH (pH 8.0), 1 mM cysteine or 2 mM selenocysteine (Secys), 10 mM MgCl2, 5 mM of each KOAc and KCl, 4 mM ATP, 22.5 μM tRNA, 10 μM CysRS, 20% DMSO, 25 mM DTT, 5 M ZnCl₂, 0.005 u/μl of yeast inorganic pyrophosphatase and 50 ug/ml BSA. Prior to reaction Secys has been reduced from selenocystine (Secis) 30 min at 37° C. in the solution containing 40 mM Secis, 50 mM HepesKOH (pH 8.0), 1M DTT, pH has been brought to 7.5 by 100 mM KOH final. Cysteinylated or selenocysteinylated tRNA (Cys(Secys)-tRNA) was purified by phenol extraction, precipitated by ethanol and either conjugated with iodoacetamide- (maleimide-) compounds of interest in the reaction containing 50 mM TrisHCl (pH8.5 or 7.2 for Secys-tRNA), 100 mM NaCl, 75% DMSO, 80 uM Cys(Secys)-tRNA and 1 mM of iodoacetamide or maleimide derivatives or used directly for the recoding of AGG (Arg) and AGC (Ser) codons for cysteine in control reactions (see FIG. 19A). Due to both lower pKu and reduction potential for Selenocysteine residue as compared to cysteine, in the conjugation reaction we could obtain folds of magnitude higher yields of BPFL-tRNA conjugation products resulting in average 25% of original tRNA material taken for aminoacylation being converted to BPFL-Se-tRNA conjugate.

Cysteinylated or selenocysteinylated t7tRNAs bearing CCU-anticodon (cys-tRNA(Cys)ccu) were shown to maintain eGFP biosynthesis in concentration dependent manner in a context of translation reaction reconstituted of both all-tRNA depleted lysate and semi-synthetic tRNA complement lacking t7tRNA(Arg)ccu and primed with eGFP-coding template harbouring single AGG- or TAG-codon (see FIG. 19B,D). Addition of non-aminoacylated tRNACysccu (FIG. 19B) failed to maintain eGFP translation causing only marginal effect when the 10 uM of tRNACysccu was used compared to background translation in a negative control reaction lacking any AGG-codon suppressors (“Semi-synthetic tRNAs-1” at B). The latter suggests that the pre-translationally aminoacylated tRNACys with altered anticodon represents a simple orthogonal system.

Cysteinylated tRNACys harbouring grafted GCU-anticodon (Cys-tRNA(Cys)gcu—see FIG. 19A, C) was shown to effectively suppress two consecutive AGC-codons in a context of translation reaction with the lysate directly depleted from endogenous tRNA(Ser)GCU using KL (FIG. 19C).

### Example 5

### Macrocyclic Protein Design and Testing

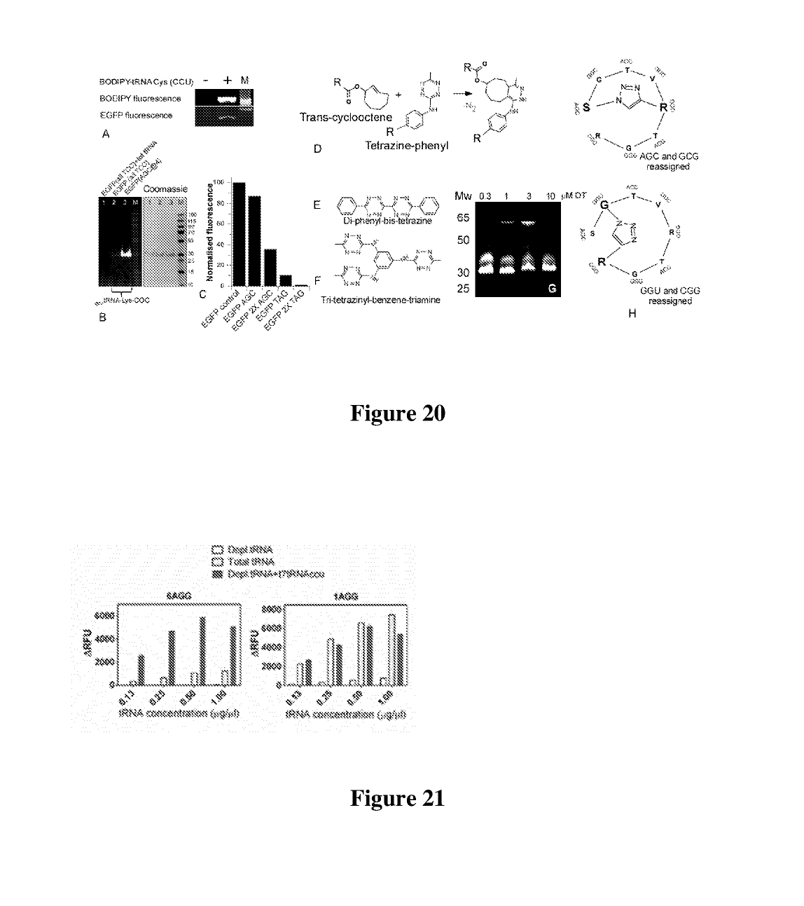

As one aspect of this project is to create peptides with intramolecular bonds, we will test several uAAs that allow homo- and hetero-condensation. A summary of one approach is provided in FIG. 20. In the simplest case, we will use Seleno-Cysteine (Se-Cys), which spontaneously forms essentially irreversible diselenium bonds under physiological conditions. The available evidence suggests that it can be charged to tRNA(Cys) by CysRS. We will test the efficiency of Se-Cys into one or multiple codons and analyse the products for spontaneous cyclisation using mass spectroscopy and endopeptidase protection assay. We will then analyse the ability of the developed system to incorporate pairs of mutually orthogonal reactive groups.

We will construct a test mRNA coding for a synthetic 10-mer peptide carrying Ser and Arg codons and a C-terminal affinity clamp tag. Initially, two AGC codons will be included in the mRNA sequence. The construct will be expressed in a cell-free system containing cuAtRNA charged with Se-Cys or amino acids carrying azide and alkyne or more reactive trans-cyclo-octene.

The Se-Cys-containing peptides are expected to cyclise spontaneously, and the extent of reaction will be determined by mass spectrometry. In the case of azide- and alkyne-modified amino acids, catalytic Cu(I) will be added into the translation mixture to induce click reaction and formation of the internal bond. In the case of cyclo-octene, functionalised amino acid cyclisation will be achieved by the addition of reagent featuring two coupled tetrazine groups (FIG. 20E). We synthesised di-tetrazine from commercially available reagents and demonstrated that it can crosslink cyclo-octene-Lys-containing EGFP molecules (FIG. 20G). Subsequently, more rigid variants featuring aromatic scaffolds functionalised by tetrazine groups will be synthesised. While the initial experiments will be carried out with reagents containing only two tetrazine groups, subsequent scaffolds with three groups will be developed (FIG. 20F). Such reagents will provide multiple crosslinks and result in highly constrained macrocyclic structures. In a control experiment, a gene lacking reassigned codons will be translated and subjected to identical conditions. The resulting products will be affinity-purified and analysed by LC-MS either in native form or following trypsin digestion to identify the new intermolecular bonds.

We will generate a library of reassigned tRNAs charged with Lys or Cys carrying such groups. Specifically, we will focus on azide- and alkyne-modified amino acids and amino acids modified with trans-cyclooctene and tetrazine. The former groups form a covalent 1,4-regioisomer in the presence of Cu(I), while the latter pair reacts spontaneously to form a heterocycle. A large panel of fluorophores and affinity reagents that can be selectively conjugated to the groups above is commercially available and will be used to assess the efficiency and selectivity of the method.

### Example 6

### Construction and Selection of Macrocyclic Peptides and Peptide Libraries

The technology disclosed herein lays the foundation for construction and selection of macrocyclic peptides. In order to integrate our macrocyclisation approach with a selection system, we will take advantage of RNA or DNA display such asCIS display. CIS display harnesses the ability of a DNA-binding protein, RepA, to exclusively bind back to its encoding DNA. The system supports effective library sizes over 10¹³ and utilises an E. coli cell-free system, which is ideally suitable for our purposes. Alternatively, this may be performed with in vitro assembled viruses or phage.

We will generate two CIS display-compatible libraries for the selection of macrocyclic binders. Our initial 10-mer library will contain available reassigned codons interlaced with tyrosines that are known to mediate the formation of high-affinity protein:protein interfaces. The second library will be computationally designed to represent 5000 selected, covering all classes of protein folds. The codon usage will be optimised to maximise the occurrence of the reassigned codons in that library. As our initial target, we will select the CNH domain of citron kinase, which has been previously identified as a targetable component of the MAP kinase pathway. The libraries cyclised through different codon reassignments will be selected on a recombinant CNH domain, and the outcomes of the selection procedures will be compared using next-generation sequencing. The peptides will then be synthesised in linear and cyclised and used in AlphaScreen and single-molecule coincidence interaction analyses. Eventually, the approach will be tested on the native Phylomer libraries of 10⁸ variants, and the performance of the libraries will be compared by sequencing of the naïve and selected libraries.

The proposed program will provide a novel approach for constructing and analysing highly diverse libraries of macrocyclic peptides. It allows large structural post-translational diversification of the resulting peptides without the need to generate a new library. This approach is likely to result in a novel platform for the generation of highly potent bioactive compounds. It will also provide scientists on the team with training in the use of bio-orthogonal chemistry and diversity-based active compound developments.

TABLE 1
The t7tRNA complement for RGS1 template. The codons in RGS1 template and the respective t7tRNAs
(see FIG. S1 for designation) are shown. The final concentrations of initiator(iMcau) and elongator
tRNAs in the cell-free translation reaction were 1.6 and 0.8 μM, respectively. For the tRNAs
encoded by 2 genetic variants such as iMcau(c1, 2), Tggu(d4, 5) and Vgac(d10, 11) the polymorphic
isoacceptor versions were mixed in ratios corresponding to their genome copy number.

| Amino acid | M | | R | G | S | I | D | T | | W | V | |
|---|---|---|---|---|---|---|---|---|---|---|---|---|
| Codons in RGS1 template | AUG | | CGG | GGC | UCC | AUC | GAC | ACC | | UGG | GUC | |
| t7tRNA | iMcau | | Rccg | Ggcc | Sgga | Igau | Dguc | Tggu | | Wcca | Vgac | |
| t7tRNA No. | c1 | c2 | a4 | b2 | c12 | b5 | a8 | d4 | d5 | d7 | d10 | d11 |
| t7tRNA concentration (μM) | 0.4 | 1.2 | 0.8 | 0.8 | 0.8 | 0.8 | 0.8 | 0.4 | 0.8 | 0.8 | 0.4 | 0.4 |

TABLE 2
The tRNA depletion efficiencies tested using one-codon RGS template. The depletion efficiency (DE.) for specific
tRNAs corresponding to their cognate codons (codon) was calculated based on the peptide expression levels in tRNA-
depleted lysate programmed by one-codon template and various tRNA mixtures using the formula shown in FIG. 11.

| aa. | Codon | DE. | aa. | Codon | DE. | aa. | Codon | DE. | aa. | Codon | DE. |
|---|---|---|---|---|---|---|---|---|---|---|---|
| Phe | UUU | 90% ± 1% | Ser | UCU | 68% ± 1% | Tyr | UAU | 91% ± 1% | Cys | UGU | 84% ± 2% |
| Phe | UUC | 81% ± 2% | Ser | UCC | 79% ± 2% | Tyr | UAC | 85% ± 1% | Cys | UGC | 37% ± 3% |
| Leu | UUA | 86% ± 1% | Ser | UCA | 92% ± 1% | Stop | UAA | | Stop | UGA | |
| Leu | UUG | 95% ± 1% | Ser | UCG | 94% ± 5% | Stop | UAG | | Trp | UGG | 31% ± 5% |
| Leu | CUU | 87% ± 1% | Pro | CCU | 95% ± 2% | His | CAU | 89% ± 3% | Arg | CGU | 46% ± 3% |
| Leu | CUC | 62% ± 9% | Pro | CCC | 99% ± 10% | His | CAC | 83% ± 9% | Arg | CGC | 11% ± 9% |
| Leu | CUA | 92% ± 4% | Pro | CCA | 94% ± 2% | Gln | CAA | 61% ± 2% | Arg | CGA | 9% ± 10% |
| Leu | CUG | 36% ± 4% | Pro | CCG | 86% ± 1% | Gln | CAG | 49% ± 1% | Arg | CGG | 90% ± 4% |
| Ile | AUU | 3% ± 2% | Thr | ACU | 53% ± 2% | Asn | AAU | 82% ± 2% | Ser | AGU | 64% ± 1% |
| Ile | AUC | 14% ± 10% | Thr | ACC | 44% ± 5% | Asn | AAC | 79% ± 4% | Ser | AGC | 72% ± 8% |
| Ile | AUA | 90% ± 2% | Thr | ACA | 93% ± 8% | Lys | AAA | 68% ± 1% | Arg | AGA | 78% ± 3% |
| eMet | AUG | 63% ± 3% | Thr | ACG | 87% ± 8% | Lys | AAG | 71% ± 2% | Arg | AGG | 85% ± 6% |
| Val | GUU | 25% ± 3% | Ala | GCU | 8% ± 9% | Asp | GAU | 60% ± 5% | Gly | GGU | 35% ± 1% |
| Val | GUC | 57% ± 9% | Ala | GCC | 1% ± 8% | Asp | GAC | 41% ± 8% | Gly | GGC | 13% ± 7% |
| Val | GUA | 68% ± 4% | Ala | GCA | 16% ± 3% | Glu | GAA | 68% ± 8% | Gly | GGA | 68% ± 8% |
| Val | GUG | 66% ± 7% | Ala | GCG | 19% ± 3% | Glu | GAG | 53% ± 12% | Gly | GGG | 62% ± 4% |

TABLE 3
DNA oligonucleotides (oligoDNAs) used for affinity
purification specific native tRNAs from the total
native tRNA mixture.

| | | Modifi- |
|---|---|---|
| Oligo name | Sequence | cations |
| OligoDNA1- | GACAGGCAGGTATTCTAACCGACTGAA | 3′-amine |
| Asp-amine | (SEQ ID NO: 8) (SEQ ID NO: 80) | |
| OligoDNA1- | TTAACAGTCCGCCGTTCTACCGACT | 3′-amine |
| Asn-amine | (SEQ ID NO: 9) (SEQ ID NO: 81) | |
| OligoDNA1- | GTGAAAGGGCGGTGTCCTGGGCCTCT | 3′-amine |
| Glu-amine | (SEQ ID NO: 10) (SEQ ID NO: 82) | |
| OligoDNA1- | TTATCAGGGGTGCGCTCTAACCACCT | 3′-amine |
| Ile-amine | (SEQ ID NO: 11) (SEQ ID NO: 83) | |
| OligoDNA2- | CTCCTCTGACTGGACTCGAACCAGTGACAT | 3′-amine |
| Asn-amine | (SEQ ID NO: 12) (SEQ ID NO: 84) | |
| OligoDNA1- | GACAGGCAGGTATTCTAACCGACTGAA | 3′-biotin- |
| Asp-biotin | (SEQ ID NO: 8) (SEQ ID NO: 85) | TEG |
| OligoDNA1- | GACAGGCAGGTATTCTAACCGACTGAA | 3′-thiol |
| Asp-thiol | (SEQ ID NO: 8) (SEQ ID NO: 86) | |
| OligoDNA1- | GTGAAAGGGCGGTGTCCTGGGCCTCT | 3′-biotin- |
| Glu-biotin | (SEQ ID NO: 10) (SEQ ID NO: 87) | TEG |

OligoDNA1 and oligoDNA2 are complementary to the target tRNA sequences either spanning the D-arm down to the anticodon loop or the acceptor stem down to variable loop, respectively.
The 3′-end of the oligonucleotides were modified with amine, biotin or thiol to facilitate their immobilization on the matrix.
TEG denotes triethylene glycol.

TABLE 4
Codon composition of sGFP_T1 and its semi-synthetic tRNA complement

| aa. | Codons | t7tRNA | codon number | tRNA ratio | aa. | Codons | t7tRNA | codon number | tRNA ratio |
|---|---|---|---|---|---|---|---|---|---|
| Ala | GCU/GCC | Aggc | 0 | 0 | iMet | 1AUG | Mcau1 | 1 | 0.5 |
| | 7GCG | Augc | 7 | 3.5 | | | Mcau2 | | 1.5 |
| | 1GCA | Augc | 1 | 0.5 | Ile | AUA | Icau1 | 0 | 0 |
| Arg | CGU/1CGC/CGA | Racg | 1 | 1 | | | Icau2 | | 0 |
| | 6CGG | Rccg | 6 | 6 | Met | 5AUG | Mcau3 | 5 | 5 |
| | 1AGG | Rccu | 1 | 1 | Phe | 10UUC/2UUU | Fgaa | 12 | 8 |
| | AGA | Rucu | 0 | 0 | Pro | 3CCG | Pcgg | 3 | 3 |
| Asn | 10AAC/3AAU | Nquu* | 13 | 6.5 | | 3CCC | Pggg | 3 | 3 |
| Asp | 13GAC/5GAU | Dguc | 18 | 9 | | 4CCA/CCU | Pugg | 4 | 4 |
| Cys | 2UGC | Cgca | 2 | 2 | Ser | 8UCG | Scga | 8 | 8 |
| Gln | 7CAG | Qcug | 7 | 7 | | 1AGC/AGU | Sgcu | 1 | 1 |
| | CAA | Quug | 0 | 0 | | 1UCC/UCU | Sgga | 1 | 1 |
| Glu | 13GAA/3GAG | Esuc* | 16 | 8 | | UCA | Suga | 0 | 0 |
| Gly | 3GGG | Gccc | 3 | 1.5 | Thr | 8ACG | Tcgu1 | 8 | 4 |
| | 2GGU/5GGC | Ggcc | 7 | 3.5 | | | Tcgu2 | | 4 |
| | 12GGA | Gucc | 12 | 6 | | 5ACC/ACU | Tggu1 | 5 | 2.5 |
| His | 8CAC/2CAU | Hgug | 10 | 6 | | | Tggu2 | | 2.5 |
| Ile | 6AUC/5AUU | Igau* | 11 | 5.5 | | 5ACA | Tugu | 5 | 5 |
| Leu | 7UUG | Lcaa | 7 | 7 | Trp | 1UGG | Wcca | 1 | 1 |
| | 5CUG | Lcag1 | 5 | 5 | Tyr | 9UAC/UAU | Ygua1 | 9 | 6 |
| | | Lcag2 | 0 | 0 | | | Ygua2 | | 3 |
| | 3CUC/3CUU | Lgag | 6 | 6 | Val | 2GUC | Vgac1 | 1 | 1 |
| | UUA | Luaa | 0 | 0 | | | Vgac2 | 1 | 1 |
| | 2CUA | Luag | 2 | 2 | | 6GUA/2GUU | Vuac | 8 | 8 |
| Lys | 20AAG | Kcuu | 20 | 12 | | 8GUG | Vuac | 8 | 8 |

TABLE 5
Codon composition of sGFP_T2 and its semi-synthetic tRNA complement

| aa. | Codons | t7tRNA | codon number | tRNA ratio | aa. | Codons | t7tRNA | codon number | tRNA ratio |
|---|---|---|---|---|---|---|---|---|---|
| Ala | GCU/GCC | Aggc | 0 | 0 | iMet | 1AUG | Mcau1 | 1 | 0.5 |
| | 8GCG | Augc | 8 | 4 | | | Mcau2 | | 1.5 |
| | GCA | Augc | 0 | 0 | Ile | AUA | Icau1 | 0 | 0 |
| Arg | CGU/CGC/CGA | Racg | 0 | 0 | | | Icau2 | | 0 |
| | 7CGG | Rccg | 7 | 7 | Met | 5AUG | Mcau3 | 5 | 5 |
| | 1AGG | Rccu | 1 | 1 | Phe | 10UUC/2UUU | Fgaa | 12 | 8 |
| | AGA | Rucu | 0 | 0 | Pro | CCG | Pcgg | 0 | 0 |
| Asn | 10AAC/3AAU | Nquu* | 13 | 6.5 | | CCC | Pggg | 0 | 0 |
| Asp | 13GAC/5GAU | Dguc | 18 | 9 | | 10CCA/CCU | Pugg | 10 | 10 |
| Cys | 2UGC | Cgca | 2 | 2 | Ser | 9UCG | Scga | 9 | 9 |
| Gln | 7CAG | Qcug | 7 | 7 | | 1AGC/AGU | Sgcu | 1 | 1 |
| | CAA | Quug | 0 | 0 | | UCC/UCU | Sgga | 0 | 0 |
| Glu | 13GAA/3GAG | Esuc* | 16 | 8 | | UCA | Suga | 0 | 0 |
| Gly | GGG | Gccc | 0 | 0 | Thr | ACG | Tcgu1 | 0 | 0 |
| | GGU/GGC | Ggcc | 0 | 0 | | | Tcgu2 | 0 | 0 |
| | 22GGA | Gucc | 22 | 11 | | ACC/ACU | Tggu1 | 0 | 0 |
| His | 8CAC/2CAU | Hgug | 10 | 6 | | | Tggu2 | 0 | 0 |
| Ile | 6AUC/5AUU | Igau* | 11 | 5.5 | | 18ACA | Tugu | 18 | 12 |
| Leu | 19UUG | Lcaa | 19 | 9.5 | Trp | 1UGG | Wcca | 1 | 1 |
| | CUG | Lcag1 | 0 | 0 | Tyr | 9UAC/UAU | Ygua1 | 9 | 6 |
| | | Lcag2 | 0 | 0 | | | Ygua2 | | 3 |
| | CUC/CUU | Lgag | 0 | 0 | Val | GUC | Vgac1 | 0 | 0 |
| | UUA | Luaa | 0 | 0 | | | Vgac2 | 0 | 0 |
| | 1CUA | Luag | 1 | 1 | | GUA/GUU | Vuac | 0 | 0 |
| Lys | 20AAG | Kcuu | 20 | 12 | | 18GUG | Vuac | 18 | 10 |

TABLE 6
Codon composition of sGFP_T3 and its semi-synthetic tRNA complement

| aa. | Codons | t7tRNA | codon number | tRNA ratio | aa. | Codons | t7tRNA | codon number | tRNA ratio |
|---|---|---|---|---|---|---|---|---|---|
| Ala | GCU/GCC | Aggc | 0 | 0 | iMet | 1AUG | Mcau1 | 1 | 0.5 |
| | 7GCG | Augc | 7 | 3.5 | | | Mcau2 | | 1.5 |
| Arg | 1GCA | Augc | 1 | 0.5 | Ile | AUA | Icau1 | 0 | 0 |
| | CGU/CGC/CGA | Racg | 0 | 0 | | | Icau2 | | 0 |
| | 7CGG | Rccg | 7 | 7 | Met | 5AUG | Mcau3 | 5 | 5 |
| | 1AGG | Rccu | 1 | 1 | Phe | 10UUC/2UUU | Fgaa | 12 | 8 |
| | AGA | Rucu | 0 | 0 | Pro | 3CCG | Pcgg | 3 | 3 |
| Asn | 10AAC/3AAU | Nquu* | 13 | 6.5 | | 3CCC | Pggg | 3 | 3 |
| Asp | 13GAC/5GAU | Dguc | 18 | 9 | | 4CCA/CCU | Pugg | 4 | 4 |
| Cys | 2UGC | Cgca | 2 | 2 | Ser | 9UCG | Scga | 9 | 9 |
| Gln | 7CAG | Qcug | 7 | 7 | | 1AGC/AGU | Sgcu | 1 | 1 |
| | CAA | Quug | 0 | 0 | | UCC/UCU | Sgga | 0 | 0 |
| Glu | 13GAA/3GAG | Esuc* | 16 | 8 | | UCA | Suga | 0 | 0 |
| Gly | 3GGG | Gccc | 3 | 1.5 | Thr | 8ACG | Tcgu1 | 8 | 4 |
| | 2GGU/5GGC | Ggcc | 7 | 3.5 | | | Tcgu2 | | 4 |
| | 12GGA | Gucc | 12 | 6 | | 5ACC/ACU | Tggu1 | 5 | 2.5 |
| His | 8CAC/2CAU | Hgug | 10 | 6 | | | Tggu2 | | 2.5 |
| Ile | 6AUC/5AUU | Igau* | 11 | 5.5 | | 5ACA | Tugu | 5 | 5 |
| Leu | 7UUG | Lcaa | 7 | 7 | Trp | 1UGG | Wcca | 1 | 1 |
| | 5CUG | Lcag1 | 5 | 5 | Tyr | 9UAC/UAU | Ygua1 | 9 | 6 |
| | | Lcag2 | 0 | 0 | | | Ygua2 | | 3 |
| | 3CUC/3CUU | Lgag | 6 | 6 | Val | 2GUC | Vgac1 | 1 | 1 |
| | UUA | Luaa | 0 | 0 | | | Vgac2 | 1 | 1 |
| | 2CUA | Luag | 2 | 2 | | 6GUA/2GUU | Vuac | 8 | 8 |
| Lys | 20AAG | Kcuu | 20 | 12 | | 8GUG | Vuac | 8 | 8 |

Tables 4-6 Codon compositions of three templates for sGFP and their corresponding semi-synthetic tRNA complements. The best-performing t7tRNA (for designations see FIG. 8) for each codon as well as its ratio (%) in the semi-synthetic tRNA complement are shown. The synthetic tRNALys for AAG codon, Kcuu was constructed by U34 to C34 replacement in the anticodon of Kuuu. The specific native tRNAs for Asn, Glu and lie, marked with asterisk, were used in semi-synthetic tRNA mixture instead of their non-functional t7tRNA counterparts.

TABLE 7
Potential “orthogonal” vs “native” codon pairs derived
from the systematic analysis of specific native tRNA depletion level,
t7tRNA's decoding efficiency and their propensity for cross-recognition
for split and unsplit codon boxes. The most promising “orthogonal”
vs “native” codon pairs include codons from mixed codon family
boxes (for Ser, Arg and Leu) as well as the ones from unsplit codon
family boxes with high wobble restrictions (for Pro, Thr and Gly).

| Amino acid | “orthogonal codon” | “native codon” |
|---|---|---|
| Ser | AGC | UCU, UCC, UCA, UCG |
| | UCG | AGU, AGC, UCU, UCC, UCA |
| Arg | AGG | CGU, CGC, CGA, CGG |
| | CGG | AGA, AGG |
| Leu | UUG | CUU, CUC, CUA, CUG |
| | CUC | CUG, CUA, UUA, UUG |
| Pro | CCC | CCG |
| Thr | ACG | ACC |
| Gly | GGG | GGC |

### REFERENCES

(1) O'Donoghue, P.; Ling, J.; Wang, Y.-S.; Söll, D. Nat Chem Biol 2013, 9, 594.
(2) Liu, C. C.; Schultz, P. G. Annu Rev Biochem 2010, 79, 413.
(3) Hong, S. H.; Ntai, I.; Haimovich, A. D.; Kelleher, N. L.; Isaacs, F. J.; Jewett, M. C. ACS Synth Biol 2014, 3, 398.
(4) Wang, Y. S.; Fang, X. Q.; Chen, H. Y.; Wu, B.; Wang, Z. Y. U.; Hilty, C.; Liu, W. S. R. Acs Chem Biol 2013, 8, 405.
(5) Albayrak, C.; Swartz, J. R. Nucleic Acids Res 2013, 41, 5949.
(6) Kim, J.; Seo, M. H.; Lee, S.; Cho, K.; Yang, A.; Woo, K.; Kim, H. S.; Park, H. S. Anal Chem 2013, 85, 1468.
(7) Wu, B.; Wang, Z. Y.; Huang, Y.; Liu, W. S. R. Chembiochem 2012, 13, 1405.
(8) Wang, K. H.; Sachdeva, A.; Cox, D. J.; Wilf, N. W.; Lang, K.; Wallace, S.; Mehl, R. A.; Chin, J. W. Nat Chem 2014, 6, 393.
(9) Chen, S.; Fahmi, N. E.; Wang, L.; Bhattacharya, C.; Benkovic, S. J.; Hecht, S. M. J Am Chem Soc 2013, 135, 12924.
(10) Frankel, A.; Roberts, R. W. RNA 2003, 9, 780.
(11) Kwon, I.; Kirshenbaum, K.; Tirrell, D. A. J Am Chem Soc 2003, 125, 7512.
(12) Link, A. J.; Tirrell, D. A. Methods 2005, 36, 291.
(13) Loscha, K. V.; Herlt, A. J.; Qi, R. H.; Huber, T.; Ozawa, K.; Otting, G. Angew Chem Int Edit 2012, 51, 2243.
(14) Odoi, K. A.; Huang, Y.; Rezenom, Y. H.; Liu, W. R. Plos One 2013, 8, e57035.
(15) Ma, C.; Kudlicki, W.; Odom, O. W.; Kramer, G.; Hardesty, B. Biochemistry 1993, 32, 7939.
(16) Forster, A. C.; Tan, Z. P.; Nalam, M. N. L.; Lin, H. N.; Qu, H.; Cornish, V. W.; Blacklow, S. C. P Natl Acad Sci USA 2003, 100, 6353.
(17) Hipolito, C. J.; Suga, H. Curr Opin Chem Biol 2012, 16, 196.
(18) Grosjean, H.; de Crecy-Lagard, V.; Marck, C. Febs Lett 2010, 584, 252.
(19) El Yacoubi, B.; Bailly, M.; de Crecy-Lagard, V. Annu Rev Genet 2012, 46, 69.
(20) Jackman, J. E.; Alfonzo, J. D. Wiley Interdiscip Rev RNA 2013, 4, 35.
(21) Giege, R.; Sissler, M.; Florentz, C. Nucleic Acids Res 1998, 26, 5017.
(22) Amikura, K.; Sakai, Y.; Asami, S.; Kiga, D. ACS Synth Biol 2014, 3, 140.
(23) Kawahara-Kobayashi, A.; Masuda, A.; Araiso, Y.; Sakai, Y.; Kohda, A.; Uchiyama, M.; Asami, S.; Matsuda, T.; Ishitani, R.; Dohmae, N.; Yokoyama, S.; Kigawa, T.; Nureki, O.; Kiga, D. Nucleic Acids Res 2012, 40, 10576.
(24) Takai, K.; Takaku, H.; Yokoyama, S. Nucleic Acids Res 1996, 24, 2894.
(25) Takai, K.; Takaku, H.; Yokoyama, S. Biochem Biophys Res Commun 1999, 257, 662.
(26) Claesson, C.; Samuelsson, T.; Lustig, F.; Boren, T. Febs Lett 1990, 273, 173.
(27) Schwarz, D.; Junge, F.; Durst, F.; Frolich, N.; Schneider, B.; Reckel, S.; Sobhanifar, S.; Dotsch, V.; Bernhard, F. Nat Protoc 2007, 2, 2945.
(28) Jackson, R. J.; Napthine, S.; Brierley, I. RNA 2001, 7, 765.
(29) Chan, P. P.; Lowe, T. M. Nucleic Acids Res 2009, 37, D93.
(30) Beckert, B.; Masquida, B. Methods Mol Biol 2011, 703, 29.
(31) Fechter, P.; Rudinger, J.; Giege, R.; Theobald-Dietrich, A. Febs Lett 1998, 436, 99.
(32) Stein, V.; Alexandrov, K. P Natl Acad Sci USA 2014, 111, 15934.
(33) Huang, J.; Makabe, K.; Biancalana, M.; Koide, A.; Koide, S. J Mol Biol 2009, 392, 1221.
(34) Huang, J.; Koide, A.; Makabe, K.; Koide, S. P Natl Acad Sci USA 2008, 105, 6578.
(35) Huang, J.; Nagy, S. S.; Koide, A.; Rock, R. S.; Koide, S. Biochemistry 2009, 48, 11834.
(36) Dong, H. J.; Nilsson, L.; Kurland, C. G. J Mol Biol 1996, 260, 649.
(37) Rosenberg, A. H.; Goldman, E.; Dunn, J. J.; Studier, F. W.; Zubay, G. J Bacteriol 1993, 175, 716.
(38) Samuelsson, T.; Boren, T.; Johansen, T. I.; Lustig, F. J Biol Chem 1988, 263, 13692.
(39) Spenkuch, F.; Motorin, Y.; Helm, M. RNA Biol 2014, 11, 1540.
(40) Kruger, M. K.; Pedersen, S.; Hagervall, T. G.; Sorensen, M. A. J Mol Biol 1998, 284, 621.
(41) Himeno, H.; Hasegawa, T.; Ueda, T.; Watanabe, K.; Miura, K.; Shimizu, M. Nucleic Acids Res 1989, 17, 7855.
(42) Kirino, Y.; Yasukawa, T.; Ohta, S.; Akira, S.; Ishihara, K.; Watanabe, K.; Suzuki, T. Proc Natl Acad Sci USA 2004, 101, 15070.
(43) Agris, P. F.; Vendeix, F. A.; Graham, W. D. J Mol Biol 2007, 366, 1.
(44) Nureki, O.; Niimi, T.; Muramatsu, T.; Kanno, H.; Kohno, T.; Florentz, C.; Giege, R.; Yokoyama, S. J Mol Biol 1994, 236, 710.
(45) Sylvers, L. A.; Rogers, K. C.; Shimizu, M.; Ohtsuka, E.; Soll, D. Biochemistry 1993, 32, 3836.
(46) Shimizu, M.; Asahara, H.; Tamura, K.; Hasegawa, T.; Himeno, H. J Mol Evol 1992, 35, 436.
(47) Stuart, J. W.; Gdaniec, Z.; Guenther, R.; Marszalek, M.; Sochacka, E.; Malkiewicz, A.; Agris, P. F. Biochemistry 2000, 39, 13396.
(48) Sundaram, M.; Durant, P. C.; Davis, D. R. Biochemistry 2000, 39, 15652.
(49) Serra, M. J.; Turner, D. H. Methods Enzymol 1995, 259, 242.
(50) Agris, P. F. Nucleic Acids Res 2004, 32, 223.
(51) Yokoyama, S.; Watanabe, T.; Murao, K.; Ishikura, H.; Yamaizumi, Z.; Nishimura, S.; Miyazawa, T. Proc Natl Acad Sci USA 1985, 82, 4905.
(52) Yarian, C.; Townsend, H.; Czestkowski, W.; Sochacka, E.; Malkiewicz, A. J.; Guenther, R.; Miskiewicz, A.; Agris, P. F. J Biol Chem 2002, 277, 16391.
(53) Wohlgemuth, I.; Pohl, C.; Mittelstaet, J.; Konevega, A. L.; Rodnina, M. V. Philos T R Soc B 2011, 366, 2979.
(54) Kothe, U.; Rodnina, M. V. Mol Cell 2007, 25, 167.
(55) Sprinzl, M.; Horn, C.; Brown, M.; Ioudovitch, A.; Steinberg, S. Nucleic Acids Res 1998, 26, 148.
(56) Cantara, W. A.; Bilbille, Y.; Kim, J.; Kaiser, R.; Leszczynska, G.; Malkiewicz, A.; Agris, P. F. J Mol Biol 2012, 416, 579.
(57) Miyauchi, K.; Ohara, T.; Suzuki, T. Nucleic Acids Res 2007, 35, e24.
(58) Yokogawa, T.; Kitamura, Y.; Nakamura, D.; Ohno, S.; Nishikawa, K. Nucleic Acids Res 2010, 38, e89.
(59) Pedelacq, J. D.; Cabantous, S.; Tran, T.; Terwilliger, T. C.; Waldo, G. S. Nat Biotechnol 2006, 24, 1170.
(60) Godinic-Mikulcic, V.; Jaric, J.; Greber, B. J.; Franke, V.; Hodnik, V.; Anderluh, G.; Ban, N.; Weygand-Durasevic, I. Nucleic Acids Res 2014, 42, 5191.
(61) Shimizu, Y.; Ueda, T. Methods Mol Biol 2010, 607, 11.
(62) Tamura, K.; Himeno, H.; Asahara, H.; Hasegawa, T.; Shimizu, M. Nucleic Acids Res 1992, 20, 2335.
(63) Ogle, J. M.; Murphy, F. V.; Tarry, M. J.; Ramakrishnan, V. Cell 2002, 111, 721.
(64) Forster, A. C.; Church, G. M. Mol Syst Biol 2006, 2, 45.
(65) Goto, Y.; Katoh, T.; Suga, H. Nat Protoc 2011, 6, 779.
(66) Fechter, P.; Rudinger, J.; Giege, R.; Theobald-Dietrich, A. Febs Lett 1998, 436, 99.
(67) Sampson, J. R.; Uhlenbeck, O. C. Proc Natl Acad Sci USA 1988, 85, 1033.
(68) Noren, C. J.; Anthony-Cahill, S. J.; Suich, D. J.; Noren, K. A.; Griffith, M. C.; Schultz, P. G. Nucleic Acids Res 1990, 18, 83.
(69) Deana, A.; Celesnik, H.; Belasco, J. G. Nature 2008, 451, 355.
(70) Chan, P. P.; Lowe, T. M. Nucleic Acids Res 2009, 37, D93.
(71) Czerwoniec, A.; Dunin-Horkawicz, S.; Purta, E.; Kaminska, K. H.; Kasprzak, J. M.; Bujnicki, J. M.; Grosjean, H.; Rother, K. Nucleic Acids Res 2009, 37, D118.
(72) Bentele, K.; Saffert, P.; Rauscher, R.; Ignatova, Z.; Bluthgen, N. Mol Syst Biol 2013, 9, 675.
(73) Roberts, R. G. Plos Biol 2013, 11, e1001509.
(74) Miyauchi, K.; Ohara, T.; Suzuki, T. Nucleic Acids Res 2007, 35, e24.
(75) Yokogawa, T.; Kitamura, Y.; Nakamura, D.; Ohno, S.; Nishikawa, K. Nucleic Acids Res 2010, 38.

### Example 7

### Dual Protein Labelling Via Sense Codon and Amber Suppressor Codon

### Materials and Methods

### Materials

The non-natural amino acids, BCN (Bicyclo [6.1.0] nonyne-Lysine), TCO* (trans-Cyclooct-2-ene-L-Lysine), TCO (trans-Cyclooctene-Lysine), PrK (N-Propargyl-Lysine), were purchased from Sirius Fine Chemicals SiChem GmbH. AzF (p-azidophenylalanine) was purchased from SynChem. TAMRA DIBO Alkyne were purchased from Life Technologies Australia Pty Ltd. BODIPY® FL Iodoacetamide were from Molecular Probes®. Ethanolamine-Sepharose was prepared by coupling ethanolamine on Epoxy-activated Sepharose 6B (GE Healthcare). The Epoxy-activated sepharose 6B was washed extensively by water (˜200 ml per gram matrix) to remove the additives and then incubated with 1M ethanolamine at room temperature with gentle agitation overnight. Another wash by water was performed till pH close to 7. The resulted ethanolamine-sepharose matrix was stored as 20% slurry in buffer A (100 mM KOAc, 25 mM NaOAc (pH5.2), 0.25 mM EDTA) with 2 mM NaN₃. 8 g matrix yields 100 ml 20% slurry.

Cloning and purification of the orthogonal tRNA Synthetases

The protein sequence for AzFRS is derived from mutation 7 as described earlier³⁹. The protein sequences for PylRS variants, MbBCNRS, MbTCORS 36 and MmPylRSAF^{19,29}, were obtained from the respective papers. The gBlocks encoding codon optimized ORFs for these proteins containing 6 His-tag at N-terminus was synthesized by IDT and subcloned into NcoI-NotI double digested pOPINE plasmid. Assembly of the digested plasmid and the desired protein gene was achieved by Gibson Assembly® Master Mix (NEB). The correct constructs were identified by sequencing. Protein expression was induced with 0.5 mM isfopropyl-β-D-thiogalactopyranoside (IPTG) in Rosetta cells at 20° C. for overnight. AzFRS was purified by Ni2+ affinity chromatography using standard buffer followed by gel filtration on Superdex 200 (GE Healthcare) in PBS buffer. The MbBCNRS and MbTCORS was purified by Ni²⁺ affinity chromatography using modified binding buffer (50 mM sodium phosphate, pH 7.4, 0.5 M NaCl, 5% Glycerol (vol/vol), 10 mM MgCl2, 0.1% Tween, 1 mM DTT, 20 mM Imidazole) and elution buffer (binding buffer plus 0.5 M imidazole). The protein was further purified by gel filtration and stored in 40 mM Hepes pH7.4, 150 mM NaCl, 10 Mm MgCl2, 10% (vol/vol) glycerol, 0.5 mM TCEP, at −80° C.

### T7tRNA Synthesis

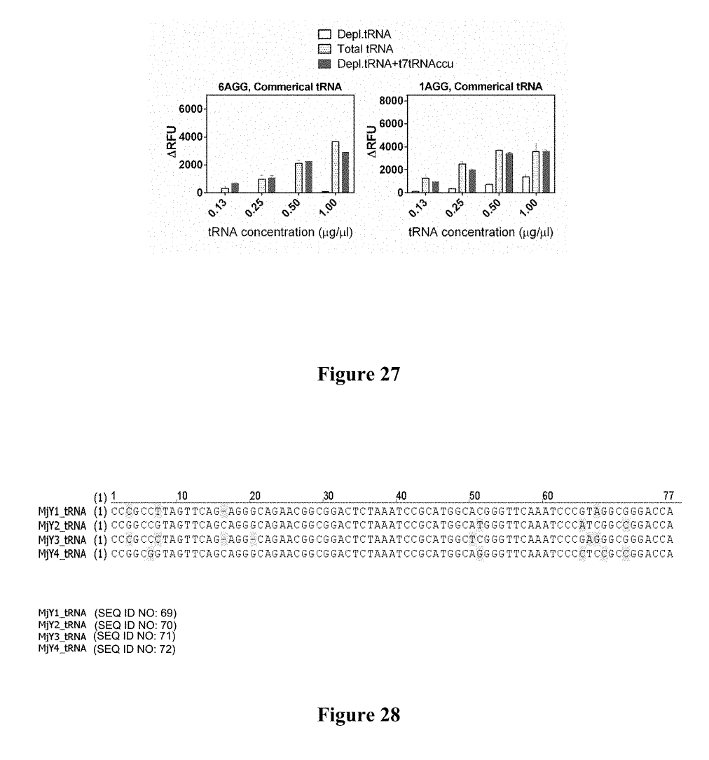

The MjY1, MjY2 and MjY3 tRNA are the best three hits identified for AzF incorporation in the earlier publication³⁷ while MjY4 tRNA with six mutations was described in a later report³⁸. The sequence alignment of these four tRNAs are shown in SI (FIG. 27). The optimized PylT tRNA is a U25C mutant³⁸. The sequence of tRNATyr cua is similar as that of wild type tRNATyr except replacing the “GUC” to “CUA” anticodon. The t7 transcripts for all the above tRNA species were prepared as described before²⁴. Basically, each DNA template, including the normal or transzyme construct, was assembled from 5 or 6 oligos (Table 8) using 3-step PCR. The PCR products were then purified by ethanol precipitation and used for run-off transcription by T7 RNA polymerase. The transcribed tRNA were purified by affinity chromatography using ethanolamine-sepharose matrix. Specifically, 2 ml transcription reaction was adjusted into buffer A condition using 10 times stock. The same volume of 20% matrix as the transcription reaction volume was added and incubated at 40 for overnight. After washing the matrix with 10 ml buffer A for 4 times, the tRNAs were eluted with 2 ml buffer B, 2M NaOAc (pH5.2), 0.25 mM EDTA, 2.5 mM Mg(OAc)₂, twice and then precipitated by ethanol.

Total tRNA Preparation

Total tRNA mixture was isolated from E. coli (Gold) strain by modified Zubay's method⁵⁹. In brief, the total tRNA was extracted from overnight culture. The cell pellet (20 g) was re-suspended in 40 ml 10 mM Mg(OAc)₂, 1 mM Tris pH7.4 buffer. The nucleoside acids were then extracted by 34.4 ml of liquefied phenol with vigorously agitated in cold room for 2 hrs. The extraction was repeated again by adding 10 ml liquefied phenol. The aqueous phase was then collected by centrifugation at 18,000 g for 30 min. RNA was precipitated with 0.05 volume 4M potassium acetate and 2 volume of 100% ethanol for overnight at −20° C. The precipitate was collected by a 10 min centrifugation at 5,000 g and re-suspended in 20 ml of 1 M cold NaCl. Stirred vigorously for 1 h in cold room to disperse the precipitate. The supernatant was collected, and extraction was repeated with 1 M NaCl. The supernatant from the two NaCl extractions was combined and precipitate by addition of 2 vol ethanol. After two repeats the crude tRNA fraction was dissolved in water.

Specific tRNA Depletion in Total tRNA Mixture

The specific tRNA species were depleted by DNA-hybridization chromatography from the total mixture as described²⁴. The DNA oligo which is complementary to the anticodon loop to acceptor stem was designed and synthesized with a 3′-amine group from IDT (Table 8). These DNA oligos were immobilized on NHS-Activated Sepharose (GE) according to the manufacturer's protocol with 100 μl of 150 μM oligo used per 100 μl resin. 200 μl of 5 μg/μl unfractionated tRNA mixture was mixed with 2×hybridization buffer (20 mM Tris-HCl (pH 7.6), 1.8M NaCl, 0.2 mM EDTA) of the same volume and then subjected for hybridization with the oligos on the matrix. For each target tRNA, 125 μl settled resin with the immobilized oligoDNA was used. The suspension of tRNA mixture and matrix were heat-denatured at 650 for 10 min. After cooling down the sample slowly at room temperature, the non-hybridized tRNAs were collected by centrifugation at slightly less than 400 μl. 40 μl of 1×hybridization buffer was then used to wash the matrix and combined with the tRNAs collected from last step. All the unbound tRNAs were combined together for ethanol precipitation and dissolved in water.

tRNA Aminoacylation and Fluorophore Conjugation

The synthetic cysteine tRNAs with CCU or CUA anticodons were prepared by t7 transcription as described above. Cysteine (Cys) or Selenocysteine (Secys) was charged on these tRNA variants by recombinant cysteinyl-tRNA synthease (CysRS, SI). Firstly, 50 mM Cys was pre-incubated with 50 mM TCEP at 37° C. for 15 min in order to convert it to fully reduced form before the aminoacylation reaction. Secys has been reduced from selenocystine (Secis) 30 min at 37° C. in the solution containing 40 mM Secis, 50 mM HepesKOH (pH 8.0), 1M DTT, pH has been brought to 7.5 by 100 mM KOH final. Meatime, tRNAs were denatured at 780 and refolded at 50 μM concentration in the presence of 5 mM Mg²⁺ slowly at room temperature to maintain their maximal activity. The aminoacylation reaction was then performed in 100 mM Hepes-KOH (pH8.0), 2 mM Cys or Secys, 2 mM TCEP or 25 mM DTT (for Secys), 10 mM MgCl2, 5 mM KCl, 5 mM KOAc, 0.1 mM CTP, 4 mM ATP, 22.5 μM t7tRNACys variant, 10 μM CysRS, 20% DMSO, 5 μM ZnCl₂, 0.005 u/μl of yeast inorganic pyrophosphatase and 50 ug/ml BSA at 370 for 1 h.

After aminoacylation, the reaction mix was diluted 4-fold into 1× buffer A for ethanolamine-sepharose purification followed by phenol extraction (residual phenol was removed by 2 subsequent extraction with 1V of chloroform/isoamyl alcohol (24:1)) and ethanol precipitation. Conjugation reaction with BODIPY® FL Iodoacetamide (BPFL-IA) was performed in 50 mM Tris-HCl (pH8.5 or 7.2 for Secys-tRNA), 80 μM Cys-tRNA, 75% DMSO, 1 mM BPFL-IA and 100 mM NaCl. Conjugated product (BPFL-tRNA) was precipitated by ethanol and dissolved in a minimal volume of tRNA buffer (0.5 mM MgCl2, 0.5 mM NaOAc pH5.0). The sample was then adjusted to 1×HPLC buffer (0.1M TEAA, 1% ACN) followed purification by HPLC on POROS® R1 10 μm Column (Applied Biosystems) using the following buffers: buffer C is 0.1M TEAA, 1% ACN; buffer D is 0.1M TEAA, 90% ACN, and 9 min run duration with 1-20% linear gradient. After HPLC purification, the BPFL-tRNA fraction was precipitated by ethanol, re-suspended in tRNA buffer and stored at −80°.

### In Vitro Protein Translation Assay

The tRNA-depleted lysate is prepared as described by ethanolamine-sepharose affinity chromatography²⁴. DNA templates with two GFP ORFs: one of optimized codon composition for eGFP (Template A) and the other of simplified codon composition for sGFP (Template B), were used. Mutations to amber or AGG-codon were introduced either by synthesizing the whole gene fragments as gBlocks by IDT to change all the 6 Arg codons to AGG simultaneously or using PCR with the proper primers to introduce mutations with less than 3 nucleotides at the same position. The obtained fragments were cloned into the pLTE or pOPINE-based plasmids following the standard Gibson assembly cloning procedure (NEB). The correct plasmids with desired mutations were purified by Midiprep Kit (Qiagen) to ensure good quality for cell free protein translation. The sequence information of all the open reading frames used here is provided in SI.

The cell-free translation reactions for GFP production were performed following the standard protocol⁶⁰ with the optimized Mg²⁺ at 10 mM concentration. 0.35 volume of depleted lysate, same as that for the standard S30 lysate, was used in the reaction. Supplementing with other compounds were noted for each experiment. GFP production was monitored on a fluorescence plate reader (Synergy) at 30° C. for 3-5 h at 485 nm excitation and 528 nm emission wavelengths.

### Protein Labeling

Expression of CaM with AzF and BP-FL in cell-free translation reaction: The pOPINE-CaM template, with TAG and AGG codon located at position 1 and 149 of CaM ORF respectively (FIG. 27; SI), were supplemented at 100 ng/l in the cell-free reaction, which contains 35% (v/v) depleted lysate, 0.5 μg/l depleted tRNA mixture, 10 μM MjY2tRNA, 10 μM AzFRS, 1 mM AzF; 10 μM BP-tRNACysccu. The reaction was performed at 32° C. for 4 h. The 200 μl cell-free reaction was dialyzed in 12-14 kDa cutoff dialysis tube against 600 ml PBS buffer for 3 h twice to get rid of AzF in the translation reaction. After dialysis, the reaction volume increased to 250 μl. Add 16 μl of 1 mM DIBO-TAMRA in the reaction to make the final concentration to 60 μM. The labeling reaction was performed at room temperature for 3 hrs.

Purification of labeled CaM on Affinity Clamp matrix: 20 μl 50% affinity clamp matrix (see SI) was added to the labelling sample after spinning the reaction mixture at 5000 rpm for 2 min. The matrix was incubated with the sample for 30 min at room temperature. The matrix was washed with 1.5 ml PBS-Triton (0.1%) buffer and then with 7.5 ml PBS and finally 3.2 ml of cleavage buffer (50 mM Tris-HCl pH8, 150 mM NaCl, 1 mM DTT). Subsequently 40 μl Precision protease (0.5 mg/ml) was added to the matrix and incubated at 16° C. for overnight. The protein was eluted with 2×75 μl of cleavage buffer.

Two separate reactions were performed to prepare single labelled CaM protein. To express CaM-BPFL, BPFL charged tRNAccu was supplemented to the cell-free translation system lacking native AGG tRNA species to decode AGG codon while 10 μM tRNATyrcua was added for decoding UAG codon to Tyr. To obtain CaM-TAMRA, AzF was incorporated into protein sequence through amber suppression with MjY2 and AzFRS into the UAG codon at first position while 5 μM synthetic tRNAArgccu was supplemented for decoding AGG codon to Arg. The AzF was then modified by DIBO-TAMRA through copper-free click chemistry as described above.

smFRET Experiment

smFRET experiments were performed in 50 mM Tris-HCl (pH 8), 150 mM NaCl, 1 mM DTT with ˜100 μM of double labeled CaM concentration. Either in the absence and presence of 2 mM Ca²⁺ or 10 mM EDTA were recorded in the smFRET experiments. 20 μl samples, loaded on a home-made silicone plate (SYLGARD®) holding by a 70×80 mm coverglass, were used for each experiments. smFRET measurements were carried out at room temperature on the Zeiss LSM 710 microscope equipped with ConfoCor3 module using Apochromat 40×1.2NA water immersion objective (Zeiss). BP-FL fluorescence was excited by 488 nm laser. Emitted light passed through the pinhole and was splited into donor and acceptor components using a 565 nm dichroic mirror; the donor and acceptor signals were further filtered by 505-540 and 580-610 nm band pass filters, respectively.

The donor and acceptor signal were simultaneously recorded for 30 s, 20 repeats with 1 ms bin time in each experiments. The data from 3 experiments were combined together to obtain enough events for analysis. The leakage of donor emission into the acceptor channel of free TAMRA dye were estimated in a separate experiment with 20 μM fluorophore concentration and calculated as 14% to correct the signals before FRET analysis. A threshold was set at 20 counts for the sum of donor and acceptor signals to filter the background noise out. The FRET efficiency were calculated as [Intensity (acceptor)−leakage from donor channel]/total intensity (Intensity (acceptor)+Intensity(donor)) and plotted as a histogram. Gaussian function was used to fit the data by Origin software (OriginLab Corp.).

### Results

Creating “AGG” Blank Codon for nnAA Incorporation

We recently demonstrated almost complete depletion of native arginine isoacceptors for AGG codon from E. coli lysate²⁴. We demonstrated that this lysate failed to support the translation of a sfGFP ORF containing a single AGG-codon while addition of synthetic tRNAccu restored its translation. The main drawback of this the original approach is the requirement to formulate a semi-synthetic tRNA mixture from the individual tRNA species. We therefore decided to test an alternative approach where the target tRNA was is selectively depleted from the total native tRNA mixture by DNA-hybridization chromatography²⁵.

For this purpose the total tRNA was isolated from BL21(Gold) E. coli strain or obtained commercially. The DNA oligonucleotides complementary to nucleotides of tRNAccu/ucu were used for their chromatographic depletion from the total tRNA mixture. The translation efficiency of tRNA mixtures before and after tRNAccu/ucu depletion were tested by the ability to support translation of GFP templates with one or six AGG codons (FIG. 21 and FIG. 27). In the former case, the rest of arginine codons in the total tRNA mixture depleted of AGG-decoding isoacceptors resulted in only negligible background translation observed for 1AGG template or no detectable translation for 6AGG template (FIG. 21 and FIG. 27). Supplementing the reactions with t7tRNAccu restored translation at the level comparable to the one provided by total tRNA mixtures before depletion. We defined tRNA depletion efficiency as 1-(RFU (Depl. tRNA)/RFU(Depl. tRNA+t7tRNAccu)). Using this assessment criteria the depletion efficiency for in-house produced tRNA mixtures range from 89% to 94% while for commercially-purchased ones from 62% to 88% calculated based on 1AGG- and 6AGG-templates respectively. In-house produced tRNA mixture was used for all subsequent experiments.

Comparing the Efficiency of o-tRNA/aaRS Pairs for nnAA Incorporation

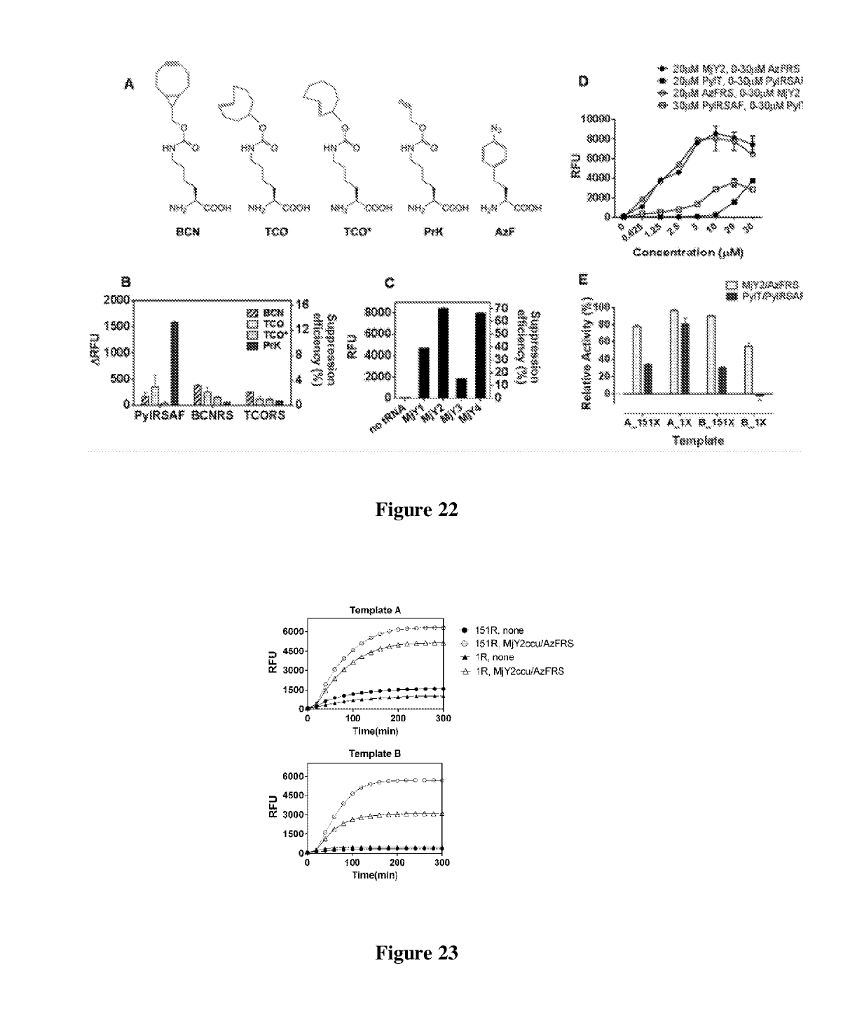

After demonstrating that AGG codon was freed for reassignment we wanted to identify the best tRNA charging system as well as the suitable nnAAs for AGG-reassignment and dual protein labelling. Therefore we decided to test the ability of the available o-tRNA/aaRS pairs based on to suppress amber codon in the in vitro translation reaction. suppression to find the best tRNA charging system as well as the suitable nnAAs for future combination with AGG-reassignment for dual protein labelling purpose. The recently developed bio-orthogonal reaction between strained alkenes/alkynes and tetrazine, known as inverse electron-demand Diels-Alder cycloaddition, displays the rate constant several orders faster higher than the earlier versions of this reaction²⁶. One of the bioorthgonal reactants, Tetrazine, is a chromophore quencher and its derivated BODIPY probes show more than 1000-fold turn-on fluorescence after conjugation with the protein²⁷, which makes the following removal of unreacted dye much easier and less stringent than the conventional dyes. Therefore, genetic incorporation of strained alkenes/alkynes would be an ideal strategy to achieve fast, selective and “turn on” protein labelling with tetrazine fluorophores. The plasticity of the active site of the PylRS enabled incorporation of a large number of nnAAs into proteins^{19,28-35} including strained alkyne and alkene, either BCN, TCO or TCO* (FIG. 22A). Three evolved PylRS variants BCNRS³⁶, TCORS³⁶ and PylRSAF¹⁹ that feature a number of unique mutations have been reported to mediate incorporation of these nnAAs in vivo. We set out to compare the efficiency of these enzymes towards several substrates in the context of an in vitro translation system.

We tested the recombinant forms of PylRS variants for their ability to support in vitro synthesis of eGFP synthesis from the template containing amber-codon at 151 position (A_151X, SI) in the presence of an evolved o-tRNA harboring U25C mutation in its anticodon stem (PylT)¹⁴ and nnAA-substrates BCN, TCO or TCO*. Another nnAA PrK (FIG. 22A) for also used for azide-coupling known to be accommodated and charged to tRNA by wt MbPylRS30 and he ORF are belonging to CGN codon family box, MmPylRS5, was also tested. Surprisingly, the efficiency of UAG suppression by BCN, TCO and TCO* was very low (FIG. 22B). Neither of these large aliphatic alkynes or alkenes could be incorporated by either of three PylRS mutants with efficiency above 5%. BCNRS showed the highest suppression efficiency with BCN and TCO* at 3% and 2%. PylRSAF demonstrated the best suppression efficiency in combination with TCO at 3%. Surprisingly, while BCNRS and TCORS essentially failed to incorporate PrK, PylRSAF charged it with 13% efficiency despite its enlarged amino acid binding pocket designed for the bulky cycloaliphatic groups confirming earlier reports²⁹. The latter confirms the promiscuity of engineered PylRS variant to various substrates reported in a comprehensive study³⁵

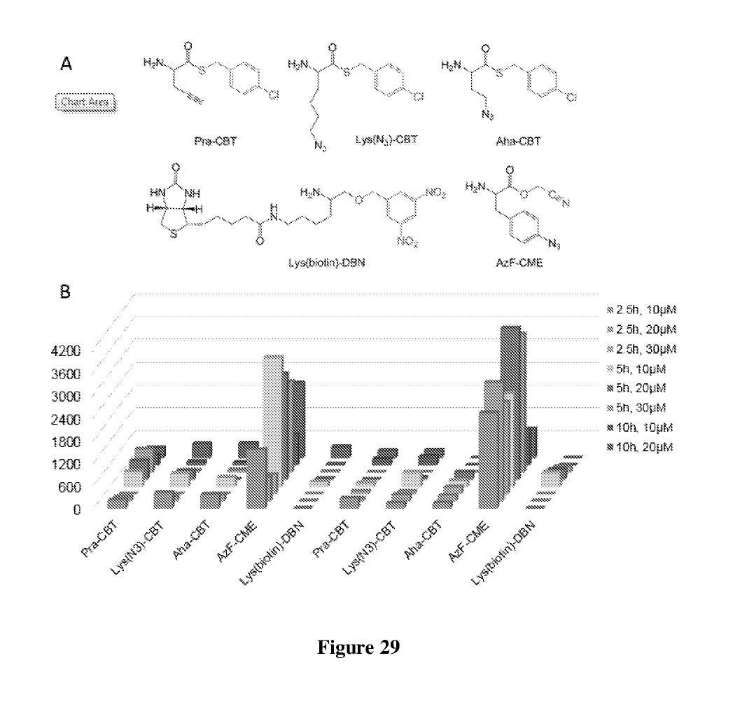

TyrRS from M. jannaschii is another widely used orthogonal tRNA charging enzyme engineered to mediate incorporation of a range of benzyl side-chain analogs including tetrazine derivative at one or multiple sites^{2,13,22}. p-Azido-L-phenylalanine (AzF, FIG. 22A) and the respective engineered version of MjTyrRS-AzFRS³⁹ were chosen based on the reports of their successful application for the amber-codon suppression in vivo and in vitro. Among tested substrates AzF showed the best amber-codon incorporation efficiency when used in combination with the flexizyme (eFx) charging system (FIG. 29). This indirectly points not only to the high acylation rate but also at the compatibility of the acylated tyrosine-analog with the EF-Tu. The importance of the latter as rate limiting step in nnAA incorporation is often underestimated.

Several optimized o-tRNAs were evolved for AzFRS in in vivo selection campaigns thus we decided to compare their activity in vitro. We found the MjY2 to support AzF incorporation with highest efficiency of 81% (FIG. 22C). It performed slightly better than MjY4 and 1.8 and 4.6 times more efficient than MjY1 and MjY3, respectively. Although MjY1 displayed the best performance in the selection experiments³⁷, was less efficient compared to MjY2 in our assay.

Following the individual characterization we compared MjY2/AzFRS/AzF and PylT/PylRSAF/PrK systematically. Firstly, the o-tRNA and aaRS were titrated into the translation reaction to determine the effect of their concentration on eGFP expression yield from the template harbouring amber codon at 151 position (A_151X). When tRNA concentration was kept constant at 20 μM, the GFP yield increased with AzFRS concentration up to 10 μM, while for PylRS it did not saturate even at 30 μM (FIG. 22D). When the concentrations of AzFRS or PylRS were kept constant at 20 μM and 30 uM, respectively, the protein yield reached plateau at 5-10 μM of MjY2 tRNA or 20 μM of PylT tRNA and declined at higher concentrations of both (FIG. 23A). The o-tRNA concentrations supporting half maximum protein yield were 1.5 μM and 8 μM for MjY2tRNA and PylT, respectively. The maximal yield for MjY system reached 8570RFU with 20 μM of MjY2 tRNA and 10 μM of AzFRS while 30 μM of both PylRSAF and PylT could support the GFP translation only to 43% of the level achieved with MjTyrRS system.

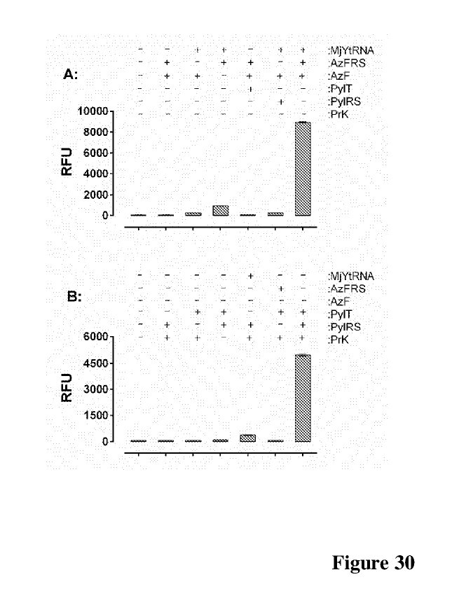

As the suppression efficiency at amber-codon is context dependence three additional GFP-coding ORFs with single TAG codon located at different positions were designed and GFP yields between two suppression systems were compared (FIG. 22D). The reactions lacking either nnAA-substrate, o-tRNA or enzyme were used to assess the level of non-specific suppression (FIG. 30; SI). E. coli T7-transcribed tyrosine tRNA with grafted CUA anticodon (EctRNATyr(cua)) known for its efficient amber codon suppression was used as a positive control. The relative activity provided by o-systems were calculated as the percentages of fluorescence in positive control (FIG. 23E).

As shown in FIG. 22E, MjY system consistently outperformed PylT system in TAG codon suppression regardless of the template context. For the template subset A the relative suppression efficiencies in both systems were found to be consistently higher for the proximal TAG-codon (position 1). Surprisingly, for the B subset, the position preference has reversed favoring the distal (position 151) over proximal amber-codon which for PylRS o-system indicated no suppression at all (FIG. 23B). Overall, the average activity of MjY o-system as compared to PylRS was 1.2-3 folds higher (without taking into account the failure of PylRS system in some case) and much less context-dependent (FIG. 22E).

### Reassigning AGG Codon to AzF

Our previous analysis findings indicated demonstrate that the orthogonal MjY tRNA charging o-system mediated mediates high level of AzF incorporation at amber-codon.

MjTyrRS missing is lacking a major anticodon-binding domain suggested indicating that the anticodon triplets were are not essential for efficient tRNA recognition40. However converting G to C in the first anticodon position (amber suppressor) or mutating all three anticodon nucleotides (ATG-suppressor) were reported to cause result in around 100 and 900-fold loss in aminoacylation efficiency, respectively 40. Since the Amber amber suppressor constructed based on Mj o-tRNA gave high suppression is very efficiency efficient, we decided to change its anticodon to test its corresponding AGG-codon suppressor by changing the anticodon to CCU thereby targeting it to AGG codon while which could potentially creating a requirement for much higher o-tRNA/enzyme concentrations in the charging reaction.

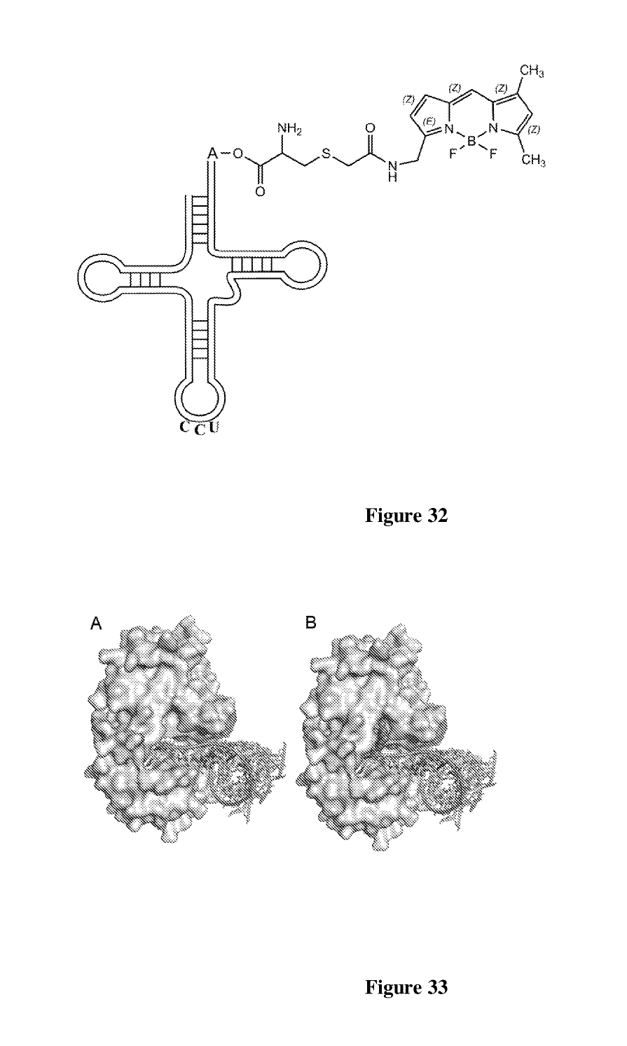

We set out to evaluate the possibility of reassigning AGG codon to AzF by combining the lysate lacking isoacceptors for AGG codon with MjY2(ccu)/AzFRS and AzF, we set out to evaluate the possibility of reassigning AGG codon to AzF. To this end we converted the single amber-codon into AGG in both codon-biased template subsets A and B (SI) while the remaining arginines in the ORF were encoded by the CGN family codons family for simultaneous monitoring of context effects (FIG. 23 and SI for sequence information). The depletion degrees of native AGG isoacceptors for A_151R, A_1R, B_151R, B_1R were found to be 75%, 80%, 93% and 84%, respectively (Equation 1). For the 151R and 1R supplementation of the reaction with MjY2(ccu)/AzFRS resulted in 4.2 and 5.2-fold increase in fluorescence for A subset while and 18.4 and 7.2-fold increase for B subsets. This indicated indicates that more than 80% of GFP protein was modified with AzF in all cases. Mass spectroscopy analysis of the translational products demonstrated that most of the protein had AzF incorporated at the AGG-defined position with very negligible a minor fraction harboring Arg at this site (FIG. 32B-C). Surprisingly even though GUA=>CCU conversion results in an anticodon different from the wt at every position and leads to a likely loss of a hydrogen bond with changing U to C3541 the AGG-suppressor here maintained high suppression activity as its amber suppressor counterpart.

Genetic encoding of Bodipy-FL nnAA derivative

The successful reassignment of AGG to AzF by MjY o-system provided us with a second orthogonal codon which once that could be combined with UAG could potentially be used for double two-site incorporation of nnAAs. However, the choice of the second nnAA chemically orthogonal to AzF is problematic due to the low incorporation efficiency of strained alkynes and alkenes and cross-reactivity between PrK as an alternative click-chemistry tag and AzF. Therefore, we chose to tested a new strategy that combines both direct incorporation of a fluorescent nnAA and a click-chemistry compatible nnAA-tag for further conjugation with a FRET-forming fluorophore. Such “pseudo-one-pot” labelling approach implying only with only one post-translational conjugation/purification derivatisation step would significantly simplify the entire is significantly simpler all previously published dual labeling procedures.

Due to their size the Fluorophore fluorophore-bearing amino acid analogs can hardly be aminoacylated by aaRS with low efficiency precluding their co-translational use co-translationally due to size limitations. Therefore co-translational supplementing supplementation of pre-charged tRNAs pre-charged with fluorescent amino acid is necessary provides an alternative approach. Several strategies have were developed for pre-charging tRNA with the nnAAs. One involves ligation of chemically prepared aminoacylated dinucleotide to a truncated tRNA lacking 3′-CA dinucleotide⁴². This method involving multi-step protocols is technically challenging. In an alternative approach flexizyme, a tRNA acylation ribozyme, was developed to charge nnAAs on tRNAs in vitro⁴³. Although in principle this approach is straightforward, its relies on multi-step chemical synthesis and the charging efficiency varies significantly towards different nnAAs and It is expensive as multi-step chemical synthesis that requires a copious amount of fluorescent dye and the dye bulkiness can likely cause steric hindrance preventing the correct orientation of the reactive groups. Another approach involves functional group coupling to the enzymatically aminoacylated tRNA through the reactive amino acid side chain such as a sulfhydryl group of cysteine44 or ε-amino group of lysine residues⁴⁵. We chose to use cysteine residue as the conjugation tag providing theas its modification results in the shortest aliphatic spacer as well as lack of editing domains in its corresponding cysteinyl-tRNA synthetase (CysRS)^{46,47} Out of a wide range of commercially available sulfhydryl-reactive fluorescent compounds we chose to use the smallest Bodipy FL fluorophore (BPFL) iodoacetamide. As improper accommodation of esterified nnAA by EF-Tu or its engineered forms quite often poses a barrier for its efficient incorporation either at the level of ternary complex formation or dissociation of nnAA-tRNA on the ribosome⁴⁸, thermodynamic compensation hypothesis demands compatibility between tRNA-body and esterified amino acids for productive incorporation⁴⁹. We therefore performed in silico analysis of EF-Tu structure to establish the acceptable linker length and fluorophore size. For instance while the commercially available BPFL attached to tRNA(Lys) amino-ester backbone through the 8-mer spacer performed visually well in protein labelling, the affinity for EF-Tu of otherwise identical BPFL-pyrrolyl analog was 30-fold lower than of wt Lys-tRNA(Lys)⁴⁸. The efficiency of incorporation of the biotin attached through 17-mer spacer could be rescued by swapping to tRNA(Ala) body tightly binding EF-Tu⁵⁰. As it seemed difficult from above to predict how ring size and spacer length would influence binding of esterified nnAA to EF-Tu we set out to roughly estimate the capacity of EF-Tu towards accommodating the BPFL/cysteinyl-tRNA conjugate where 5-mer spacer separates BPFL ring from the amino-ester backbone (FIG. 32, SI). In line with this, we manually fitted BPFL in the place of Trp into the known structure⁵¹ adjusting the spacer length to 5-mer as well (FIG. 33, SI). In the obtained model BPFL was occupying the pocket without any significant steric clashes in a slightly different orientation as Trp, and moreover there seemed to remain some room for spacer extension. Even though, from the model BPFL-cysteine derivative seemed most likely to be accommodated by EFTu at least to some extent thus contributing some negative free binding energy, this energy is very hard to rank relatively to Cys itself or aromatic amino acid residues whose distribution by affinity is very broad^{52,53}. Therefore for conjugation with BPFL we chose to keep wt T-stem and natural affinity of tRNA(Cys) to EFTu ranging dependent on used algorithm from moderate to tight reciprocally compensating weak to moderate binding of Cys.

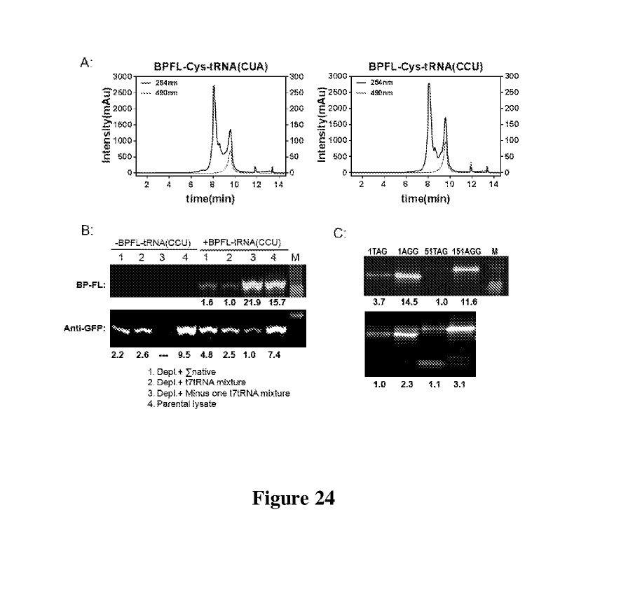

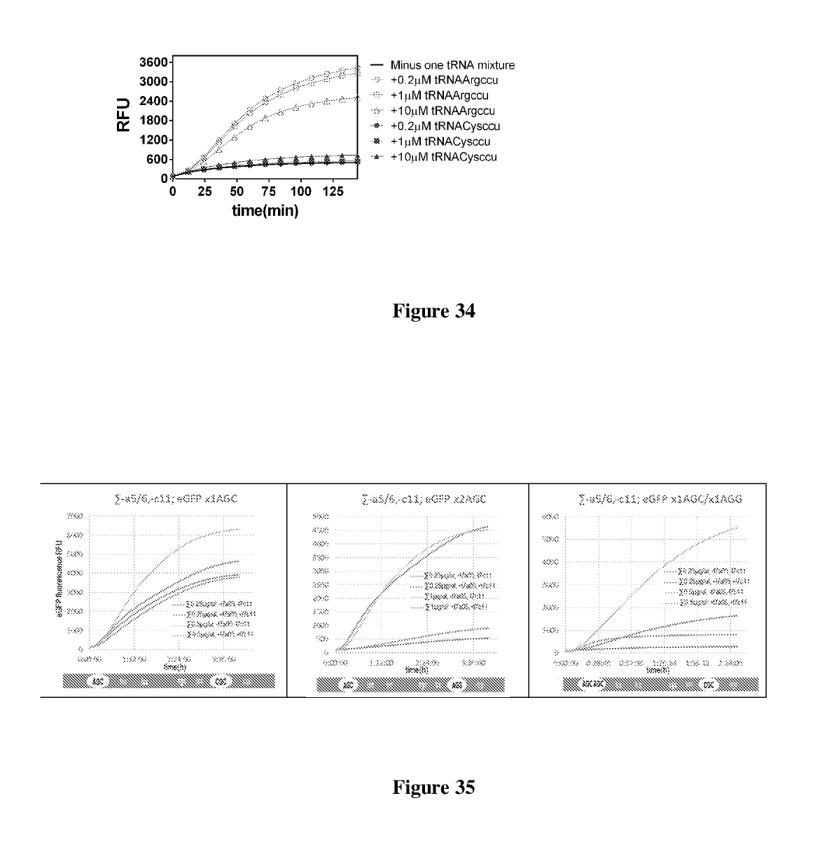

t7tRNA(Cys) variants harboring either CUA or CCU anticodons were aminoacylated and conjugated to BPFL and yielding BPFL-cys-tRNA and the conjugation products were then purified by HPLC at with ˜15-20% final yield (FIG. 24A). Next we supplemented introduced the purified BP-cys-tRNAccu in the cell free into in vitro translation mixture lacking isoacceptors for AGG reaction with semi-synthetic t7tRNA mixture lacking the respective isoacceptors for co-translational single-site protein labelling via AGG-suppression using sGFP_T2 template²⁴. The reaction was primed by eGFP template with a single AGG codon and the The yield of labelled protein versus total protein produced in both undepleted “parental” lysate and tRNA-depleted lysate programmed by different tRNA mixtures (FIG. 24B) was compared. The yield of labelled protein was increased 1.4 times while the relative labelling efficiency was 10 times higher in tRNA-reconstituted system as compared to wt lysate (FIG. 24B, lane 3 and 4 with BP-tRNA). The BPFL-tRNA less favorably competes with the native tRNA for decoding the AGG codon in normal lysate due to the presence of the native AGG-isoacceptors even though they are in low abundance abundant in the native tRNA pool⁵⁴. Almost no sGFP was produced in the cell-free system with semi-synthetic t7tRNA complement lacking the AGG isoacceptor (FIG. 24B, lane 3 without BP-tRNA). The expression of sGFP could be restored upon addition of BPFL-Cys-tRNAccu, indicating that virtually all expressed polypeptide incorporated BP-fluorophore. We concluded that BP-FL charged tRNA was compatible with the host translational machinery including both the elongation factor and the ribosome. Cys-tRNAccu discharged from nnAA during translation underwent negligible co-translational recharging with cysteine (FIG. 34, SI). The system's orthogonality is probably due to relatively low concentration of endogenous CysRS and highly reduced kcat/Km of the AGG-suppressor entailing resulting in unfavorable competition with endogeneous endogenous wt tRNA(Cys) since CysRS relies on anticodon (in particular: G35C36) as is an important identity element in tRNA recognition by CysRS recognition⁴⁷.

We compared Amber amber and AGG codon-mediated BP-FL incorporation efficiency by using on four templates, B_1X(151X) and B_1R(151R), harboring either single amber or AGG codon at the 1st and 151st position of GFP ORF (FIG. 24C, SI for detailed discussion). The translation reactions reconstituted of from tRNA depleted lysate and total tRNA mixture lacking AGG isoacceptors were supplemented with purified BPFL-cys-tRNAs suppressors and primed with their respective templates. AGG suppression gave 1.8 and 4 times consistently higher BP-FL incorporation, 1.8 and 4 times on the 1st and 151st position respectively, than amber suppression. This indicates the less low competition from the native tRNA isoacceptors for AGG codon compared to that of release factor for mediated translation termination on amber codon. We concluded that BP-FL charged tRNA was more compatible with AGG in the codon contexts we tested here.

### Dual Protein Labelling Via Combination of AGG and Amber Codon Reassignment

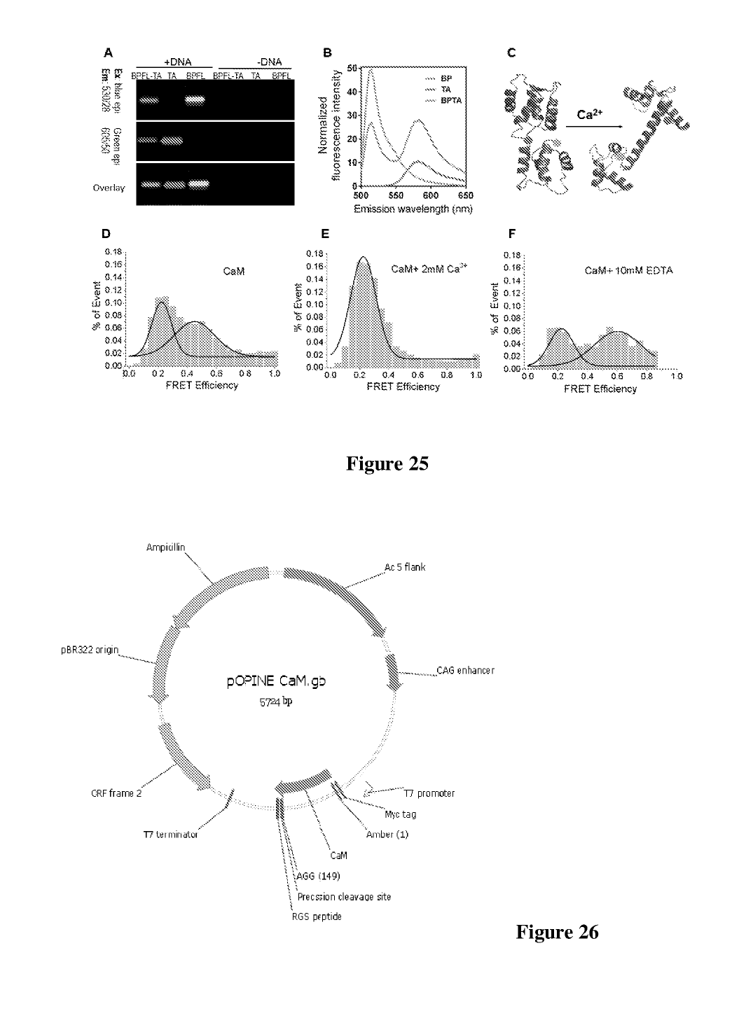

As described in the introduction the current strategies for site-specific dual protein labeling with the FRET-forming dye pair rely on introduction of two bio-orthogonal groups which are genetically incorporated into the protein sequence followed by two respective conjugation reactions to another groups delivering the FRET forming fluorophores^{7,19}. The requirement for carefully control reactivity of the functional groups in these nnAAs and fluorophores makes this approach technically challenging. Since BP-FL could be incorporated into protein sequence co-translationally, installation of the 2nd fluorophore would become more straightforward without many with little restrictions in the choice of the second genetically encoded nnAAs. We tested this approach by synthesizing Calmodulin (CaM), a regulator protein of cellular functions, with a bio-orthogonal reactive group (AzF) and fluorophore-bearing amino acid (BPFL-Cys) installed at 1st and 149th position via amber- and AGG-codon reassignment respectively. TAMRA, a that forms a FRET forming dye with pair with BPFL, was then introduced into protein directly via copper-free click chemistry. This reaction can be processed carried out either on pure the protein or in the cell-free reaction context and avoid the side effect of protein aggregation or oxidation caused by metal catalyst which was required in the copper-catalyzed azide-alkyne huisgen cycloaddition (CuAAC). Fluorescence gel scanning demonstrates confirmed successful installation of two FRET probes in CaM protein (FIG. 25A, first lane).

Excitation at 488 nm of double labelled CaM revealed two distinct peaks on in the fluorescence emission spectra (FIG. 25B). The 512 nm peak corresponds to emission of BP-FL while the other peak at 575 nm reflects emission of TAMRA. In contrast, CaM proteins monolabeled with BP-FL or TAMRA show low emission at 575 nm in their fluorescence emission spectra when excited at 488 nm. The concentration of CaM protein single-labelled with TAMRA single-labelled CaM protein was pre-adjusted to equalize its maximum emission fluorescence to that of double labelled protein when excited at 543 nm (acceptor excitation wavelength). When both samples were excited at 488 nm, the emission fluorescence at 575 nm of double labeled protein was much higher than that of TAMRA single-labelled protein. This indicates the energy transfer from donor fluorophore BP-FL to acceptor fluorophore TAMRA in double labeled CaM and confirms the successful double labelling.

To demonstrate the application of smFRET in protein conformational change analysis, we titration the dual labelled CaM protein with Ca²⁺ and EDTA (FIG. 25D-F). The dual-labelled CaM was purified from the cell free translation in a buffer condition without Ca²⁺. Its smFRET histogram shows two peaks, one is centered at 0.2 while the other at 0.55. They represent two conformations in the purified sample which we assume one is in Ca²⁺ free and the other in Ca²⁺-binding form. The cell-free expressed CaM is probably contaminated with trace amount of calcium and triggered the conformational change of some CaM protein in the sample. After adding 2 mM Ca²⁺ in the sample, the second peak disappeared and emerged with the first one. This confirmed that the first peak at around 0.2 is the calcium bound form. When adding EDTA in the sample, the first peak decreased obviously which indicated some Ca²⁺ was stripped from the CaM protein. This data is consistent with the structural information that the two fluorophores in the 1st and 149th position of CaM are closer in Ca²+-free form (high FRET efficiency) than Ca2+-binding form (FIG. 25C).

### Discussion

We describe here aa new strategy for reassigning Arg AGG sense codon to an uAA in E. coli in vitro translation system. To achieve that we took advantage of the recently developed E. coli translation system depleted of the endogenous tRNAs24. We now developed novel procedure for removing selected tRNAs from the total tRNA mixture by DNA-hybridization chromatography. Unlike the previously reported reconstitution approach using semi-synthetic tRNA complement, the developed tRNA-depletion method is straightforward and efficient. We were able to achieve almost complete depletion of the native isoacceptors for AGG codon making it suitable for reassignment. Without tRNAccu supplementation, no protein translation was observed using GFP template with 6AGG codons while only minimal GFP expression was observed on the template containing one 1AGG codon template. Upon addition of synthetic tRNAccu, the GFP protein expression increased to the levels comparable to that of the wild type system. This validates sense codon reassignment strategy and opens up a way for creation of potentially large number of new orthogonal codons.

The open nature of cell-free translation system enables the direct control over the concentrations and the identities of its components. We took advantage of this feature to compare several orthogonal tRNA/aaRS systems including Pyl and MjTyr system in their ability to support incorporation of nnAAs into in vitro translated protein. We evaluated several reported PylRS variants by their ability to mediate incorporation of nnAAs including BCN, TCO, TCO* and PrK. We were particularly interested in the first three non-natural amino acids due to their high reactivity and bioorthogonality²⁶. Surprisingly, none of them appeared to be good substrates for the tested PylRS variants. These results revealed an unexpected inconsistency with the in vivo data on UAG suppression where efficient incorporation of these nnAAs was confirmed by MS analysis^{19,36}. In contrast, PylRSAF could efficiently decode UAG codon with PrK with relatively high efficiency and can be further modified through copper-catalysed click chemistry. This indicates that the aminoacyl-tRNA synthetases evolved by in vivo selection may not be optimal especially for thesefor large aliphatic alkynes or alkenes. PylRS variants in general are multi-substrate enzymes with high degree of substrate-induced inter-domain dynamics rearrangements. therefore Therefore mutagenesis of the substrate binding pocket can on another hand entail may result in reduction of overall amino-acylation kinetic by several hundred fold³⁵. We observed precipitation of these PylRS based enzymes during the purification step. The instability of these PylRS variants in buffer conditions causes an extra barrier for in vitro application although magnesium, high concentration salt and glycerol could help maintain these proteins in soluble form to some extent. This is consistent with some in vivo data which suggested PylRS were poorly expressed, prone to misfolding and required optimizations of codon usage and expression conditions in E. coli for nnAA incorporation¹⁴.

When analysing MjTyr system we found that MjY2 was best most efficient in coding AzF functionalities, although the MjY1 was screened identified as the most efficient in vivo³⁷ and MjY4(tRNAOptCUA) was employed in the most recent work³⁸. The evolutionary pressure ensures that in the native system all aa-tRNAs show similar affinity towards elongation factor (EF-Tu) by combining amino acid side chains with different tRNA bodies^{49,55}. Therefore, in order to achieve the best nnAA incorporation efficiency, it is desirable to optimize the wild type o-tRNA sequence to work in concert with the chosen target nnAAs to achieve optimal affinity to EF-Tu.

Further, we compared the amber codon context on decoding efficiency by the best combinations of two orthogonal translational systems, MjY2/AzFRS/AzF and PylT/PylRSAF/PrK. MjTyr system performed consistently better than Pyl system in all the four codon contexts we tested here. The relative efficiency of MjTyr system ranged from 55% to 97% in decoding UAG codon. In contrast, the suppression efficiency of PylT/PylRS/PrK varied and in some cases failed to mediate GFP translation. These results are consistent with the previous findings that some positions of ORFs are suppressed easier than others^{56,57} and indicate that context effect is reduced when highly efficient o-aaRS/tRNA pairs are used.

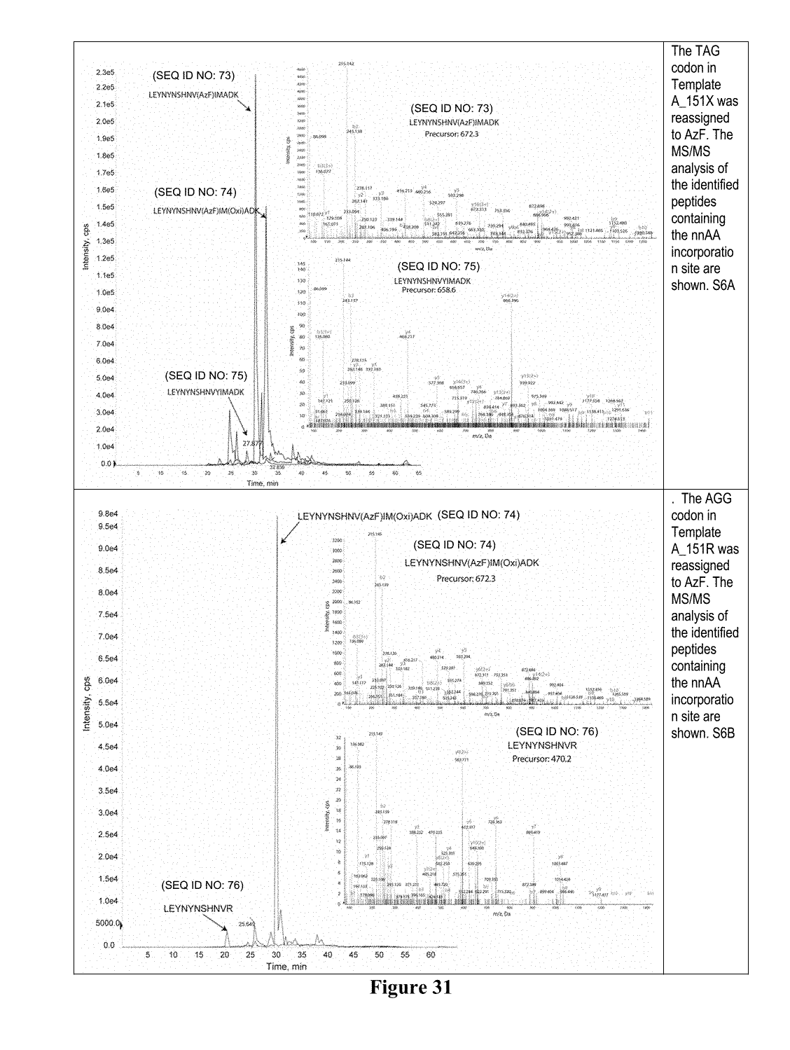

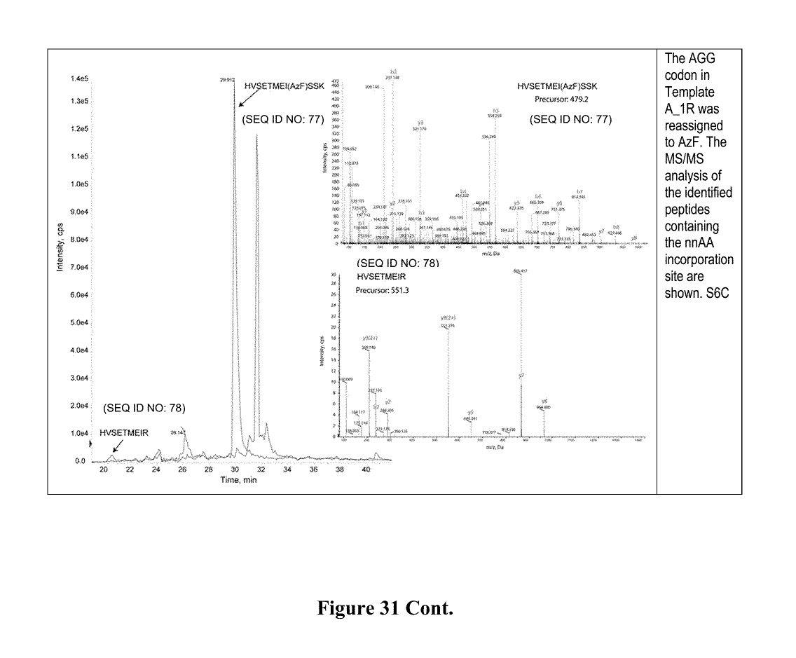

High orthogonality of heterogeneous tRNA/aaRS/nnAA is a key issue determining the specificity and fidelity of codon reassignment. Our results demonstrated that orthogonality of Pyl suppression system is higher than that of MjY system in E. coli (FIG. 30) as in the latter case we observed incorporation of the canonical aromatic amino acids, Tyr, Phe, Trp in the absence of AzF. However, after supplementing AzF in translation system, the canonical amino acid mis-incorporation became negligible (FIG. 31) suggesting that the MjTyrRS optimized for a specific nnAA had low efficiency in accepting other substrates. Although broad substrate specificity has advantages for incorporation of nnAAs with similar structures, it also increases misincorporation when more than one nnAAs needs to be incorporated. The ratio of each matured tRNAs and their cognate aaRSs are evolutionary balanced to ensure the translational fidelity and efficiency.

We demonstrated MjY suppression system not confined to amber suppression but is also well compatible for with AGG-reassignment to AzF when combined with our newly developed cell free system lacking the AGG-native isoacceptors. The low sensitivity of MjTyrRS to the tRNA anticodon triplets of MjTyrRS enables its future use for reassigning reassignment to other sense codons. Besides AzF, AGG codon could be reassigned to BP-FL fluorophore directly, which would significantly simplify the labelling procedure, by using precharged BPFL-Cys-tRNAccu. The discharged tRNAccu was is poor substrate of CysRS due to the altered anticodon and underwent negligible co-translational recharging with cysteine. This maintained maintains the system's fidelity. Further optimization of the tRNA body in the context of esterified BPFL-Cys for interaction with sequence to enhance its compatibility with the esterified BPFL-Cys to be better accommodated by EF-Tu would increase the decoding efficiency.

We took advantage of the above described observations to develop a new approach for construction of for site-selective proteins protein labelled labelling with two FRET-forming probes. To avoid difficulties associated with balancing the selectivity of two labelling reactions We we decided to incorporate one fluorophore co-translationally in the protein sequence to avoid difficulties associated with balancing the selectivity of two labelling reactions. We combined the most efficient MjTyr suppression system with the BPFL-pre-charged tRNA to reassign amber and AGG codon, respectively. Followed by copper-free click chemistry, TAMRA dye was further installed into protein sequence by copper-free click chemistry through the reaction with azido-group accomplished within several minutes. By using this strategy, we site-specifically installed BP-FL and TAMRA into calmodulin protein. Single molecule FRET analysis shows dramatically change of its conformation upon calcium binding to calcium confirming the efficient and site selective dye incorporation. The developed approach potentially allows creation of large number of orthogonal codons and their modification with versatile functional groups. Furthermore this approach can be directly transferred to eukaryotic cell-free systems. This sets our approach apart from methods based on the use of fully reconstituted in vitro translation systems such as PURE system which although being relatively easily produced for prokaryotic translational machinery brings in almost unmanageable complexity when transferred to eukaryotic systems. Yet our recent analysis demonstrated that E. coli cell-free system could express only 10% of human cytosolic proteins in non-aggregated form while eukaryotic systems performed much better⁵⁸. Therefore transferring our approach onto a eukaryotic system would enable smFRET analysis of complex multidomain eukaryotic proteins and protein complexes currently poorly accessible to this method.

### Supplementary Information (SI)

Nucleotide sequences encoding proteins
(1) Temple A (eGFP template with GenBank number of KJ541667.1, based on pLTE vector)
(SEQ ID NO: 13)
ATGACAGTAATGTATAAAGTCTGTAAAGACATTAAACACGTAAGTGAAACCATGGAGATCXXXTCGAGCAAGG
GCGAGGAGCTGTTCACCGGGGTGGTGCCCATCCTGGTCGAGCTGGACGGCGACGTAAACGGCCACAAGTTCA
GCGTGTCCGGCGAGGGCGAGGGCGATGCCACCTACGGCAAGCTGACCCTGAAGTTCATCTGCACCACCGGCAA
GCTGCCCGTGCCCTGGCCCACCCTCGTGACCACCCTGACCTACGGCGTGCAGTGCTTCAGCCGCTACCCCGACC
ACATGAAGCAGCACGACTTCTTCAAGTCCGCCATGCCCGAAGGCTACGTCCAGGAGCGCACCATCTTCTTCAAG
GACGACGGCAACTACAAGACCCGCGCCGAGGTGAAGTTCGAGGGCGACACCCTGGTGAACCGCATCGAGCTG
AAGGGCATCGACTTCAAGGAGGACGGCAACATCCTGGGGCACAAGCTGGAGTACAACTACAACAGCCACAAC
GTCTATATCATGGCCGACAAGCAGAAGAACGGCATCAAGGTGAACTTCAAGATCCGCCACAACATCGAGGACG
GCAGCGTGCAGCTCGCCGACCACTACCAGCAGAACACCCCCATCGGCGACGGCCCCGTGCTGCTGCCCGACAA
CCACTACCTGAGCACCCAGTCCGCCCTGAGCAAAGACCCCAACGAGAAGCGCGATCACATGGTCCTGCTGGAG
TTCGTGACCGCCGCCGGGATCACTCTCGGCATGGACGAGCTATACAAGGAGCAGAAGCTGATCTCGGAGGAG
GATCTGCAAGCTTGTCGACCTCTAGAGGATCCCCGGGGCTAA
Template A contains STS protein; eGFP construct and rnyc tag from N- to C-term. It is used
as a parental template to prepare the following derivatives:
(a) GFP with 6AGG codons (All 6 Arg codons in this construct are changed to AGG);
(b) A_1X: TAG codon was introduced at the N-term of eGFP construct (the 1st red codon)
(c) A_151X: TAT(Tyr codon) was changed to TAG codon at the 151st position of eGFP
construct (the 2^{nd} red codon)
(d) A1R: AGG codon was introduced at the N-term of eGFP construct (the 1st red codon)
(e) A_151R: TAT(Tyr codon) was changed to AGG codon at the 151st position of eGFP
construct (the 2^{nd} red codon)
(2) Template 6 (sGFP template, based on pOPINE vector with GenBank number of EF372397.1).)
(SEQ ID NO: 14)
ATGACAGAACAGAAGTTGATCTCGGAAGAAGATTTGXXXTCGAAGGGAGAAGAATTGTTTACAGGAGTGGTG
CCAATCTTGGTGGAATTGGACGGAGATGTGAACGGACACAAGTTCTCGGTGCGTGGAGAAGGAGAAGGAGAC
GCGACAAACGGAAAGTTGACATTGAAGTTCATCTGCACAACAGGAAAGTTGCCAGTGCCATGGCCAACATTGG
TGACAACATTGACATACGGAGTGCAGTGCTTTTCGCGTTACCCAGATCACATGAAGCGTCATGACTTCTTCAAGT
CGGCGATGCCAGAAGGATACGTGCAGGAACGTACAATCTCGTTCAAGGACGACGGAACATACAAGACACGTG
CGGAAGTGAAGTTCGAAGGAGACACATTGGTGAATCGTATTGAATTGAAGGGAATCGACTTCAAGGAGGATG
GAAACATTTTGGGACACAAGTTGGAGTACAACTTCAACTCGCACAACGTGXXXATCACAGCGGACAAGCAGAA
GAACGGAATCAAGGCGAACTTCAAGATTCGTCACAACGTGGAGGACGGATCGGTGCAGTTGGCGGATCACTAC
CAGCAGAATACACCAATTGGAGATGGACCAGTGTTGTTGCCAGACAATCACTACTTGTCGACACAGTCGGTGTF
GTCGAAGGACCCAAACGAAAAGCGTGACCACATGGTGTTGTTGGAATTTGTGACAGCGGCGGGAATTACACAT
GGAATGGACGAATTGTACAAGGGAGGATTGGAGGTGTTGTTCCAGGGACCAGGACGTGGATCGATCGACACA
TGGGTGTGATGATGA
Template B contains myc tag, sGFP construct, prescission cleavage site and RGS peptide tag
from N-to C-term. It is used as a parental template to prepare the folIowing derivatives:
(a) B1X: TAG codon was introduced at the N-term o1 sGFP construct (the 1st red codon)
(b) B_151X: TAT(Tyr codon) was changed to TAG codon at the 151st position of sGFP construct
(the 2^{nd} red codon)
(c) b_1R: AGG codon was introduced at the N-term of sGFP construct (the 1st red codon)
(d) B15IR: TAT(Tyr codon) was changed to AGG codon at the 151st position of sGFP construct
(the 2^{nd} red codon
(3) sGFP_T2
(SEQ ID NO: 15)
ATGTCGAAGGGAGAAGAATTGTTCACAGGAGTGGTGCCAATCTTGGTGGAATTGGACGGAGATGTGAACGGA
CACAAGTTCTCGGTGCGGGGAGAAGGAGAAGGAGACGCGACAAACGGAAAGTTGACATTGAAGTTCATCTGC
ACAACAGGAAAGTTGCCAGTGCCATGGCCAACATTGGTGACAACATTGACATACGGAGTGCAGTGCTTTTCGC
GGTACCCAGATCACATGAAGCGGCATGACTTCTTCAAGTCGGCGATGCCAGAAGGATACGTGCAGGAACGGAC
AATCTCGTTCAAGGACGACGGAACATACAAGACAAGGGCGGAAGTGAAGTTCGAAGGAGACACATTGGTGAA
TCGGATTGAATTGAAGGGAATCGACTTCAAGGAGGATGGAAACATTTTGGGACACAAGTTGGAGTACAACTIC
AACAGCCACAACGTGTACATCACAGCGGACAAGCAGAAGAACGGAATCAAGGCGAACTTCAAGATTCGGCAC
AACGTGGAGGACGGATCGGTGCAGTTGGCGGATCACTACCAGCAGAATACACCAATTGGAGATGGACCAGTG
TTGTTGCCAGACAATCACTACTTGTCGACACAGTCGGIGTTGTCGAAGGACCCAAACGAAAAGCGGGACCACAT
GGTGCTATTGGAATTTGTGACAGCGGCGGGAATTACACATGGAATGGACGAATTGTACAAG

Template sGFP_T2¹ only contains open reading frame for sGFP protein. Arginines in this protein are encoded by AGG codon at 108 position (shown in red) while the rest of them are by CGG codon.

(4) CaM
(SEQ ID NO: 16)
ATGGAACAGAAGCTGATTTCCGAAGAGGATCTGGGATAGGATCAGCTGA
CGGAAGAGCAGATCGCGGAATTCAAGGAAGCGTTCTCCCTGTTCGACAA
GGACGGAGACGGAACGATTACGACGAAGGAACTGGGAACGGTTGATGCG
TTCCCTGGGACAGAATCCGACGGAAGCGGAGCTGCAGGACATGATCAAC
GAAGTGGATGCGGACGGAAACGGAACGATTGACTTCCCGGAATTCCTGA
CGATGATGTGCGCGTAAGATGAAGGATACGGACTCCGAAGAGGAAATCC
GTGAAGCGTTTCGTGTGTTCGACAAGGATGGAAATGGATACATTTCCGC
GGCGGAGCTGCGTCACGTGATGACGAACCTGGGAGAGAAGCTGACGGAC
GAAGAGGTGGACGAAATGATTCGTGAAGCGGACATTGACGGAGACGGAC
AGGTGAATTACGAAGAGTTCGTGCAGATGATGACGGCGAGGGGAGGACT
GGAGGTGCTGTTCCAGGGACCGGGACTGGATCCATCGACACGTGGGTGT
GATGATGA
CaM template contains myc tag, CaM protein,
prescission cleavage site and RGS peptide tag
from N-to C-term as shown in the vector map.

### Materials and Methods

### Recombinant Cysteinyl-tRNA Synthease Production:

The gene fragment for CysRS was amplified by colony PCR on E. coli BL21(DE3) strain and cloned into pET28 vector with the C-terminal His-tag and purified by standard affinity chromotography. Protein expression was induced with 0.5 mM isfopropyl-β-D-thiogalactopyranoside (IPTG) in BL21(DE3)RIL cells at 20° C. for overnight. CysRS was purified by Ni2+ affinity chromatography using standard buffer followed by gel filtration on Su-perdex 200 (GE Healthcare) in buffer: 50 mM Hepes-KOH, pH8, 150 mM NaCl and 1 mM DTT.

### Affinity Clamp Matrix Preparation:

C-terminal Cys were added into sequence of the affinity clamp protein (pdz-fibronectin Fusion Protein, PDB: 3CH8_A). The purified protein with C-terminal Cys was immobilized on Iodoacetyl Gel (UltraLink®) using manufacture's protocol. tRNA charging by flexizyme for amber suppression: The acylation reaction were performed as described before⁶². 1 volume of 250 μM o-tRNA was mixed with the same volume of 50 μM Fx (250 μM) and 3 volume of water. The mixture was then heated at 950 for 5 min with constant agitation at 1200 rpm and cooled down slowly at room temperature. After adding 1 volume of 0.5M Hepes (pH7.9) and 2 volume of 3M MgCl₂, the mixture was put on ice for 2 min and followed by addition of 2V of nnAA substrate (100 mM) (in 100% DMSO or 100% ACN). The reactions were incubated at 10° with agitation for the given periods of time (2.5, 5 or 10 h). Flexizyme acylation reactions were stopped and used to prime the translational reactions.

### MS Analysis:

The modified proteins were purified on nano-trap matrix and then eluted with 1×SDS PAGE loading buffer. After running the gel, the desired bands were cut and digested with trypsin. The resulting peptides were then analyzed by LC-MS/MS.

### Results and Discussion

Although a commercial tRNA mixture demonstrated ˜2-fold less efficiency than that produced herein in supporting translation of 1AGG sGFP template, it displayed 3-fold higher activity towards supporting the translation of eGFP template with 6AGG codons at saturating concentrations (FIG. 21 and FIG. 27). This indicates a greater proportion of native rare AGG-codon isoacceptors in the commercial mixture. Addition of t7tRNAccu to the depleted in-house produced tRNA mixture resulted in 5-fold increase in translation activity as compared to original total tRNA mixture (FIG. 21). This confirmed our assumption that at appropriate age of cell culture the fraction of low abundant tRNA species for decoding AGG codon is biased favorably for codon reassignment purposes.

In order to ensure translational fidelity, the exogenous tRNA/aaRS pairs should be orthogonal to the endogenous ones. We evaluated the translational fidelity of MjY2/AzFRS/AzF and PylT/PylRSAF/PrK in E. coli cell free translation system. Template A_151X was translated in the cell free reaction by supplementing with different combinations of o-tRNA/aaRS/nnAA. ForMjTyr system, exclusion of either tRNA or aaRS, no protein was translated. However, when AzF was excluded from the reaction around 10% of protein was produced. It indicates that AzFRS could charge MjY2tRNA with canonical amino acids for amber suppression. We also found that with the increasing concentration of AzFRS the ability to charge MjtRNA with canonical amino acids increased (data not shown). To exam this issue, the proteins produced without and with AzF were purified by affinity chromatography on nano-trap matrix and load for SDS-PAGE gel. The corresponding band was cut and digested using trypsin. The resulting peptides were then analyzed using LC-MS/MS to assess the identity of the amino acid at the desired position. MS spectrum analysis of the GFP products without AzF supplementation showed some aromatic amino acids, Phe, Tyr and Trp, were incorporated on TAG codon (data not shown). In the GFP151AzF sample, wild type peptide was negligible and more than 80% of the protein contained AzF (FIG. 32A). The AzF incorporation could also be confirmed by following TAMRA incorporation through DIBO-TAMRA.

The orthogonality of PylT/PylRSAF pair is better than MjY2/AzFRS pair. Exclusion either nnAA, PylT or PylRS, no detectable fluorescence was observed. However, when supplementing the reaction with MjY2 tRNA, the protein yield increased slightly which indicated MjYtRNA may be misacylated by PylRS with Prk. The ratio of each matured tRNAs and their cognate aaRSs are evolutionary balanced to ensure the translational fidelity and efficiency. Therefore, when the 21st and 22nd o-tRNA/aaRS pairs are introduced into the translation system, care must be taken to avoid cross-recognition.

We compared Amber and AGG codon-mediated BP-FL incorporation efficiency by using four templates harbouring either single AGG or single amber codon at the 1^{st} and 151^{st} GFP ORF (FIG. 25C). The yield of BP-FL labelled protein on 1TAG was 3.7 times that of 151TAG while on 1AGG was 1.2 times that of 151AGG. The suppression efficiency on the 1^{st} position is consistently higher than the 151^{st} position for both amber and AGG codon. The relative total protein yield was represented by band intensity on the western blot. If we assume the full length protein for 151TAG suppression (FIG. 25C, lane 3, anti-GFP) was directly due to BP-FL incorporation, then the band intensity ratio of BP-FL/anti-GFP (upper band) should be considered as 100% labelling efficiency. The fluorescence/total protein ratio for 1AGG suppression (FIG. 25C, lane 2) was obviously higher than that for 151TAG which indicates more than 100% labelling efficiency and is not possible. Therefore, some unspecific incorporation was in the full length band for 151TAG suppression. It not only consists of products with BP-FL, but with some other non-fluorescent amino acid. The highest probability is Cys which was incorporated into protein through the discharged tRNACyscua at the late stage of protein translation derived from the BPFL-tRNA suppressor. This unspecific incorporation is context dependent since the fluorescence/total protein ratio for 1TAG (3.7/1=3.7) is around 8 times higher than that for 151TAG (1/(1+1.1)=0.48). Therefore it is not straightforward to estimate the AGG labelling efficiency from the TAG labelling efficiency derived from the ratio of full length/(full length and truncation). However, the labelling efficiency here for 1TAG:1AGG:151TAG:1AGG is 4:7:1:4. AGG suppression gave consistently higher BP-FL incorporation, 1.8 and 4 times on the 1^{st} and 151^{st} position respectively, than amber suppression. This indicates the less competition from the native tRNA isoacceptors for AGG codon compared to that of release factor for termination on amber codon. We concluded that BP-FL charged tRNA was more compatible with AGG codon in the contexts we tested here.

TABLE 8
Oligos for synthetic tRNA preparation and depletion of native isoacceptors

| tRNA name | Oligo name | Sequence |
|---|---|---|
| MjY1 | T7 | GTAATACGACTCACTATAGG (SEQ ID NO: 17) |
| | F1 | GTAATACGACTCACTATAGGGAGACTAAGGCGGGCTGATGAGTCCGTGAGGACGAAACGG (SEQ ID NO: 18) |
| | F2 | CCGTGAGGACGAAACGGTACCCGGTACCGTCCCCGCCTTAGTTCAGAGGGCAGAACGGCG (SEQ ID NO: 19) |
| | R1 | GAACCCGTGCCATGCGGATTTAGAGTCCGCCGTTCTGCCCTC(SEQ ID NO: 20) |
| | R2 | TGGTCCCGCCTACGGGATTTGAACCCGTGCCATGCGGA (SEQ ID NO: 21) |
| | R3 | TGGTCCCGCCTACGGGATTT (SEQ ID NO: 22) |
| MjY2 | F1 | GTAATACGACTCACTATAGGGAGACTACGGCCGGCTGATGAGTCCGTGAGGACGAAACGG (SEQ ID NO: 23) |
| | F2 | CCGTGAGGACGAAACGGTACCCGGTACCGTCCCGGCCGTAGTTCAGCAGGGCAGAACGGC(SEQ ID NO: 24) |
| | R1 | GAACCCATGCCATGCGGATTTAGAGTCCGCCGTTCTGCCCTGC(SEQ ID NO: 25) |
| | R2 | TGGTCCGGCCGATGGGATTTGAACCCATGCCATGCGGA (SEQ ID NO: 26) |
| | R3 | TGGTCCGGCCGATGGGATTT (SEQ ID NO: 27) |
| MjY3 | F1 | GTAATACGACTCACTATAGGGAGACTAGGGCGGGCTGATGAGTCCGTGAGGACGAAACGG (SEQ ID NO: 28) |
| | F2 | CCGTGAGGACGAAACGGTACCCGGTACCGTCCCCGCCCTAGTTCAGAGGCAGAACGGCGG (SEQ ID NO: 29) |
| | R1 | GAACCCGAGCCATGCGGATTTAGAGTCCGCCGTTCTGCCTC(SEQ ID NO: 30) |
| | R2 | TGGTCCCGCCCTCGGGATTTGAACCCGAGCCATGCGGA (SEQ ID NO: 31) |
| | R3 | TGGTCCCGCCCTCGGGATTT (SEQ ID NO: 32) |
| MjY4 | F1 | GTAATACGACTCACTATAGGGAGACTACCGCCGGCTGATGAGTCCGTGAGGACGAAACGG (SEQ ID NO: 33) |
| | F2 | CCGTGAGGACGAAACGGTACCCGGTACCGTCCCGGCGGTAGTTCAGCAGGGCAGAACGGC(SEQ ID NO: 34) |
| | R1 | GAACCCCTGCCATGCGGATTTAGAGTCCGCCGTTCTGCCCTGC(SEQ ID NO: 35) |
| | R2 | TGGTCCGGCGGAGGGGATTTGAACCCCTGCCATGCGGA (SEQ ID NO: 36) |
| | R3 | TGGTCCGGCGGAGGGGATTT (SEQ ID NO: 37) |
| PylT(u25c) | F1 | CTAATACGACTCACTATAGGAAACCTGATCATGTAGATCGAACGGAC(SEQ ID NO: 38) |
| | R1 | TAACCCGGCTGAACGGATTTAGAGTCCGTTCGATCTACATG (SEQ ID NO: 39) |
| | R2 | TGGCGGAAACCCCGGGAATCTAACCCGGCTGAACGG (SEQ ID NO: 40) |
| | R3 | TGGCGGAAACCCCGGGAATC(SEQ ID NO: 41) |
| tRNATyr(cua) | F1 | GTAATACGACTCACTATAGGTGGGGTTCCCGAGCGGCCAAAGGGAGCAGA (SEQ ID NO: 42) |
| | R1 | GAACCTTCGAAGTCGATGACGGCAGATTTAGAGTCTGCTCCCTTTGGCCGCTC(SEQ ID NO: 43) |
| | R2 | TGGTGGTGGGGGAAGGATTCGAACCTTCGAAGTCGATGACGGCAGA (SEQ ID NO: 44) |
| | R3 | TGGTGGTGGGGGAAGGATTC(SEQ ID NO: 45) |
| RNACys(ccu) | F1 | GTAATACGACTCACTATAGGCGCGTTAACAAAGCGGTTATGTAGCGGA (SEQ ID NO: 46) |
| | R1_ccu | GAACCGGACTAGACGGATTAGGAATCCGCTACATAACCGCTT (SEQ ID NO: 47) |
| | R2 | TGGAGGCGCGTTCCGGAGTCGAACCGGACTAGACGGA (SEQ ID NO: 48) |
| | R3 | TGGAGGCGCGTTCCGGAGTC(SEQ ID NO: 49) |
| tRNACys(cua) | R1 cua | GAACCGGACTAGACGGATTTAGAATCCGCTACATAACCGCTT (SEQ ID NO: 50) |

| | |
|---|---|
| FortRNAArg(CCU)depletion | TGTCCCCTGCAGGAATCGAACCTGCAATTA(3′-amine) (SEQ ID NO: 51) (SEQ ID NO: 88) |
| FortRNAArg(UCU)depletion | AGAAGGTCGTTGCTCTATCCAACTGAGCTA(3′-amine) (SEQ ID NO: 52) (SEQ ID NO: 89) |

Note:
2 forward (T7, F) and 3 reverse oligonucleotides (R1, R2, R3) are used for assembly of standard tRNA template while 3 forward (T7, F1, F2) and 3 reverse oligonucleotides for generating a self-processing Hammerhead ribozyme (HHRz) fusion constructs(REF).
Oligos for depletion of native isoacceptors in the total mixture contain 3′-amine for immobilization on the NHS-activated matrix.

### REFERENCES

(1) Crick, F. H. J Mol Biol 1968, 38, 367.
(2) Liu, C. C.; Schultz, P. G. Annu Rev Biochem 2010, 79, 413.
(3) O'Donoghue, P.; Ling, J.; Wang, Y. S.; Soll, D. Nature chemical biology 2013, 9, 594.
(4) Odoi, K. A.; Huang, Y.; Rezenom, Y. H.; Liu, W. R. Plos One 2013, 8, e57035.
(5) Wan, W.; Huang, Y.; Wang, Z. Y.; Russell, W. K.; Pai, P. J.; Russell, D. H.; Liu, W. R. Angew Chem Int Edit 2010, 49, 3211.
(6) Neumann, H.; Wang, K. H.; Davis, L.; Garcia-Alai, M.; Chin, J. W. Nature 2010, 464, 441.
(7) Wang, K. H.; Sachdeva, A.; Cox, D. J.; Wilf, N. W.; Lang, K.; Wallace, S.; Mehl, R. A.; Chin, J. W. Nat Chem 2014, 6, 393.
(8) Wu, B.; Wang, Z.; Huang, Y.; Liu, W. R. Chembiochem 2012, 13, 1405.
(9) Krishnakumar, R.; Prat, L.; Aerni, H. R.; Ling, J. Q.; Merryman, C.; Glass, J. I.; Rinehart, J.; Soll, D. Chembiochem 2013, 14, 1967.
(10) Lee, B. S.; Shin, S.; Jeon, J. Y.; Jang, K. S.; Lee, B. Y.; Choi, S.; Yoo, T. H. ACS chemical biology 2015, 10, 1648.
(11) Mukai, T.; Yamaguchi, A.; Ohtake, K.; Takahashi, M.; Hayashi, A.; Iraha, F.; Kira, S.; Yanagisawa, T.; Yokoyama, S.; Hoshi, H.; Kobayashi, T.; Sakamoto, K. Nucleic acids research 2015, 43, 8111.
(12) Zeng, Y.; Wang, W.; Liu, W. S. R. Chembiochem 2014, 15, 1750.
(13) Dumas, A.; Lercher, L.; Spicer, C. D.; Davis, B. G. Chem. Sci. 2015, 6, 50.
(14) Chatterjee, A.; Sun, S. B.; Furman, J. L.; Xiao, H.; Schultz, P. G. Biochemistry 2013, 52, 1828.
(15) Sasmal, P. K.; Carregal-Romero, S.; Han, A. A.; Streu, C. N.; Lin, Z.; Namikawa, K.; Elliott, S. L.; Koster, R. W.; Parak, W. J.; Meggers, E. Chembiochem 2012, 13, 1116.
(16) Iwane, Y.; Hitomi, A.; Murakami, H.; Katoh, T.; Goto, Y.; Suga, H. Nat Chem 2016.
(17) Quast, R. B.; Mrusek, D.; Hoffmeister, C.; Sonnabend, A.; Kubick, S. Febs Lett 2015, 589, 1703.
(18) Michalet, X.; Weiss, S.; Jager, M. Chem Rev 2006, 106, 1785.
(19) Nikic, I.; Plass, T.; Schraidt, O.; Szymanski, J.; Briggs, J. A. G.; Schultz, C.; Lemke, E. A. Angew Chem Int Edit 2014, 53, 2245.
(20) Sachdeva, A.; Wang, K. H.; Elliott, T.; Chin, J. W. Journal of the American Chemical Society 2014, 136, 7785.
(21) Albayrak, C.; Swartz, J. R. Nucleic acids research 2013, 41, 5949.
(22) Loscha, K. V.; Herlt, A. J.; Qi, R.; Huber, T.; Ozawa, K.; Otting, G. Angewandte Chemie 2012, 51, 2243.
(23) Wan, W.; Tharp, J. M.; Liu, W. R. Biochimica et biophysica acta 2014, 1844, 1059.
(24) Cui, Z.; Stein, V.; Tnimov, Z.; Mureev, S.; Alexandrov, K. J Am Chem Soc 2015, 137, 4404.
(25) Yokogawa, T.; Kitamura, Y.; Nakamura, D.; Ohno, S.; Nishikawa, K. Nucleic acids research 2010, 38, e89.
(26) Lang, K.; Chin, J. W. ACS chemical biology 2014, 9, 16.
(27) Carlson, J. C.; Meimetis, L. G.; Hilderbrand, S. A.; Weissleder, R. Angewandte Chemie 2013, 52, 6917.
(28) Mukai, T.; Kobayashi, T.; Hino, N.; Yanagisawa, T.; Sakamoto, K.; Yokoyama, S. Biochem Biophys Res Commun 2008, 371, 818.
(29) Yanagisawa, T.; Ishii, R.; Fukunaga, R.; Kobayashi, T.; Sakamoto, K.; Yokoyama, S. Chem Biol 2008, 15, 1187.
(30) Nguyen, D. P.; Lusic, H.; Neumann, H.; Kapadnis, P. B.; Deiters, A.; Chin, J. W. J Am Chem Soc 2009, 131, 8720.
(31) Lang, K.; Davis, L.; Torres-Kolbus, J.; Chou, C. J.; Deiters, A.; Chin, J. W. Nat Chem 2012, 4, 298.
(32) Lang, K.; Davis, L.; Wallace, S.; Mahesh, M.; Cox, D. J.; Blackman, M. L.; Fox, J. M.; Chin, J. W. J Am Chem Soc 2012, 134, 10317.
(33) Seitchik, J. L.; Peeler, J. C.; Taylor, M. T.; Blackman, M. L.; Rhoads, T. W.; Cooley, R. B.; Refakis, C.; Fox, J. M.; Mehl, R. A. Journal of the American Chemical Society 2012, 134, 2898.
(34) Wang, Y. S.; Fang, X.; Wallace, A. L.; Wu, B.; Liu, W. R. Journal of the American Chemical Society 2012, 134, 2950.
(35) Guo, L. T.; Wang, Y. S.; Nakamura, A.; Eiler, D.; Kavran, J. M.; Wong, M.; Kiessling, L. L.; Steitz, T. A.; O'Donoghue, P.; Soll, D. Proc Natl Acad Sci USA 2014, 111, 16724.
(36) Lang, K.; Davis, L.; Wallace, S.; Mahesh, M.; Cox, D. J.; Blackman, M. L.; Fox, J. M.; Chin, J. W. Journal of the American Chemical Society 2012, 134, 10317.
(37) Guo, J. T.; Melancon, C. E.; Lee, H. S.; Groff, D.; Schultz, P. G. Angew Chem Int Edit 2009, 48, 9148.
(38) Young, T. S.; Ahmad, I.; Yin, J. A.; Schultz, P. G. J Mol Biol 2010, 395, 361.
(39) Chin, J. W.; Santoro, S. W.; Martin, A. B.; King, D. S.; Wang, L.; Schultz, P. G. Journal of the American Chemical Society 2002, 124, 9026.
(40) Steer, B. A.; Schimmel, P. J Biol Chem 1999, 274, 35601.
(41) Tsunoda, M.; Kusakabe, Y.; Tanaka, N.; Ohno, S.; Nakamura, M.; Senda, T.; Moriguchi, T.; Asai, N.; Sekine, M.; Yokogawa, T.; Nishikawa, K.; Nakamura, K. T. Nucleic acids research 2007, 35, 4289.
(42) Hecht, S. M.; Alford, B. L.; Kuroda, Y.; Kitano, S. The Journal of biological chemistry 1978, 253, 4517.
(43) Goto, Y.; Katoh, T.; Suga, H. Nat Protoc 2011, 6, 779.
(44) Gubbens, J.; Kim, S. J.; Yang, Z.; Johnson, A. E.; Skach, W. R. Rna 2010, 16, 1660.
(45) Johnson, A. E.; Woodward, W. R.; Herbert, E.; Menninger, J. R. Biochemistry 1976, 15, 569.
(46) Newberry, K. J.; Hou, Y. M.; Perona, J. J. Embo J 2002, 21, 2778.
(47) Hauenstein, S.; Zhang, C. M.; Hou, Y. M.; Perona, J. J. Nat Struct Mol Biol 2004, 11, 1134.
(48) Mittelstaet, J.; Konevega, A. L.; Rodnina, M. V. Journal of the American Chemical Society 2013, 135, 17031.
(49) Schrader, J. M.; Chapman, S. J.; Uhlenbeck, O. C. Proc Natl Acad Sci USA 2011, 108, 5215.
(50) leong, K. W.; Pavlov, M. Y.; Kwiatkowski, M.; Ehrenberg, M.; Forster, A. C. Rna-a Publication of the Rna Society 2014, 20, 632.
(51) Voorhees, R. M.; Schmeing, T. M.; Kelley, A. C.; Ramakrishnan, V. Science 2010, 330, 835.
(52) Asahara, H.; Uhlenbeck, O. C. P Natl Acad Sci USA 2002, 99, 3499.
(53) Asahara, H.; Uhlenbeck, O. C. Biochemistry 2005, 44, 11254.
(54) Dong, H. J.; Nilsson, L.; Kurland, C. G. J Mol Biol 1996, 260, 649.
(55) LaRiviere, F. J.; Wolfson, A. D.; Uhlenbeck, O. C. Science 2001, 294, 165.
(56) Bossi, L.; Ruth, J. R. Nature 1980, 286, 123.
(57) Pott, M.; Schmidt, M. J.; Summerer, D. ACS chemical biology 2014, 9, 2815.
(58) Gagoski, D.; Polinkovsky, M. E.; Mureev, S.; Kunert, A.; Johnston, W.; Gambin, Y.; Alexandrov, K. Biotechnology and bioengineering 2016, 113, 292.
(59) Zubay, G. J Mol Biol 1962, 4, 347.
(60) Schwarz, D.; Junge, F.; Durst, F.; Frolich, N.; Schneider, B.; Reckel, S.; Sobhanifar, S.; Dotsch, V.; Bernhard, F. Nat Protoc 2007, 2, 2945.
(61) Cui, Z.; Stein, V.; Tnimov, Z.; Mureev, S.; Alexandrov, K. Journal of the American Chemical Society 2015, 137, 4404.
(62) Goto, Y.; Katoh, T.; Suga, H. Nat Protoc 2011, 6, 779.
(63) Guo, J. T.; Melancon, C. E.; Lee, H. S.; Groff, D.; Schultz, P. G. Angew Chem Int Edit 2009, 48, 9148.
(64) Young, T. S.; Ahmad, I.; Yin, J. A.; Schultz, P. G. J Mol Biol 2010, 395, 361.

### Example 8

### Dual Protein Labelling Via Two Sense Codons

Specific Depletion of Total tRNA Mixture for AGG and AGC Native tRNA Suppressors

Combining both procedures for specific tRNA depletion based on anticodon loop specific tRNA aptamer (as per Example 2) followed by the one based on tRNA-specific DNA oligonucleotide (as per Example 7 “Specific tRNA depletion in total tRNA mixture”) we generated total tRNA mixture highly depleted for serine tRNA GCU-isoacceptor (c11) and for both arginine CCU/UCU isoaccptors (a05/06). In suppression experiment both tRNAs were shown to be depleted to a high extent (FIG. 35).

### Dual Protein Labelling Via Two Sense Codons

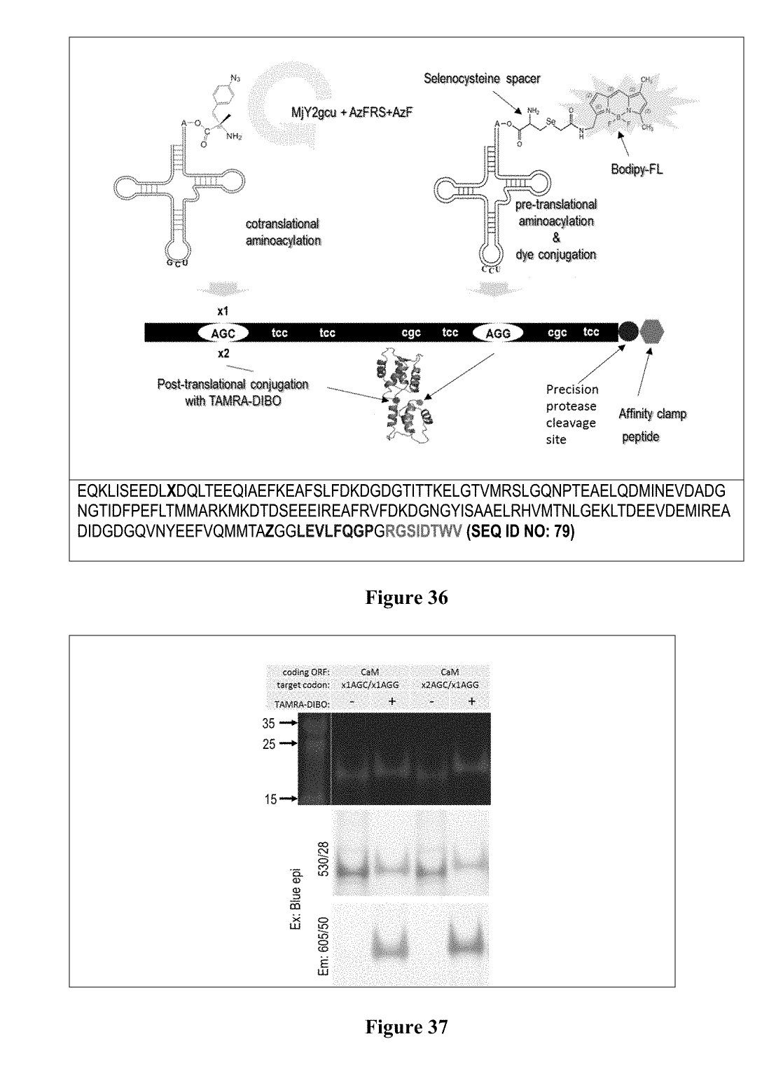

Site-specific incorporation of AzF and BP-FL into CaM and eGFP via AGC and AGG-codons in the cell-free translation reaction primed with the respective templates with different codon biases: The pOPINE-CaM templates, with single or double AGC and single AGG codon located at positions 1 (1 and 2) and 151 respectively of CaM ORF (FIG. 36), were supplemented at 100 ng/μl in the cell-free reaction, which contains 35% (v/v) depleted lysate, 0.5 μg/l depleted tRNA mixture, 12.5 μM MjY2tRNAgcu, 10 μM AzFRS, 1 mM AzF; 16 μM BP-tRNACysccu. The reaction was performed at 28° C. for 3 h. The translated product from 200 μl cell-free reaction was immobilized on Affinity-clamp resin beads (see Example 7). Resin with the immobilized translation product was washed with 25 mM Tris (pH7.5), 0.25M NaCl, 0.1% Triton X-100 buffer to remove free AzF from the translation reaction, followed by addition of DIBO-TAMRA to 1 mM final concentration in two original reaction volumes of PBS. After 1 h conjugation at RT the beads were washed to remove unconjugated TAMRA-dye and the aliqoute of beads corresponding to a protein obtained from 10 ul of translation reaction has been eluted with 95° C. heated ×2 SDS-PAGE sample buffer and resolved on SDS-PAGE (FIG. 37).

Throughout the specification, the aim has been to describe the preferred embodiments of the invention without limiting the invention to any one embodiment or specific collection of features. Various changes and modifications may be made to the embodiments described and illustrated without departing from the present invention.

The disclosure of each patent and scientific document, computer program and algorithm referred to in this specification is incorporated by reference in its entirety.

1.-28. (canceled)

29. A method of:
(i) producing a complement of tRNAs suitable for translation of a protein comprising at least one non-natural moiety, said method including the step of substituting at least one tRNA that comprises an anticodon for a natural amino acid with at least one tRNA comprising the same anticodon reassigned to a non-natural moiety, wherein the complement of tRNAs is operable to facilitate translation of an RNA which comprises a codon corresponding to the anticodon that has been reassigned to the non-natural moiety whereby the translated protein may comprise any or all of the twenty natural amino acids; or
(ii) producing a translation system suitable for translation of a protein comprising at least one non-natural moiety, said method including producing a complement of tRNAs comprising at least one tRNA that comprises an anticodon for a natural amino acid that has been reassigned to a non-natural moiety; and producing a transcribable RNA which comprises a codon corresponding to the anticodon that has been reassigned to the non-natural moiety, wherein the mRNA may be transcribed to produce a translated protein that may comprise any or all of the twenty natural amino acids; or
(iii) producing a recombinant protein comprising at least one non-natural moiety, said method including the step of translating an mRNA which comprises a codon corresponding to an anticodon of a tRNA that has been reassigned to a non-natural moiety in a complement of tRNAs comprising at least one tRNA that comprises an anticodon for a natural amino acid that has been reassigned to a non-natural moiety, wherein the translated protein may comprise any or all of the twenty natural amino acids.

30. The method of claim 29 (i), which includes one or more of the steps of: (a) depleting one or more tRNAs from a complement of tRNAs suitable for translation of a protein comprising natural amino acids; and (b) reconstituting the depleted complement of tRNAs with one or more tRNAs respectively reassigned to non-natural moieties and respectively coupled to the non-natural moieties; optionally wherein:
in step (a), substantially all tRNAs for natural amino acids are depleted from the complement of tRNAs or one or more tRNAs for natural amino acids are selectively depleted from the complement of tRNAs; and/or
in step (b), the reconstituting tRNAs comprise synthetic tRNAs, native tRNAs, or mixtures thereof.

31. The method of claim 29 (i), (ii) or (iii), wherein the reassigned anticodon is one of a plurality of different anticodons that is normally for a natural amino acid.

32. The method of claim 29 (i), (ii) or (iii), wherein the aminoacyl-tRNA is synthesized in vitro; optionally wherein:
(a) the tRNA is produced using an RNA synthetase; or
(b) the tRNA is produced using an RNA synthetase and the tRNA is aminoacylated using an RNA synthetase.

33. The method of claim 29 (i), (ii) or (iii), wherein the aminoacyl-tRNA is synthesized in vitro and the tRNA is produced using an RNA synthetase and the tRNA is aminoacylated using an RNA synthetase and the RNA synthetase is selected from PylRS or variants thereof suitable for pyrrolysine tRNA synthase-mediated aminoacylation; Methanococcus jannaschii tyrosyl-transfer RNA synthetase (Mj TyrRS) or variants thereof; Flexizyme; and a cysteinyl tRNA synthetase; optionally wherein:
(a) the tRNA comprising the reassigned anticodon is a mutant cysteine tRNA;
(b) the tRNA comprising the reassigned anticodon is a mutant cysteine tRNA and the aminoacyl-tRNA is synthesized in vitro using a cysteinyl tRNA synthase; or
(c) the tRNA comprising the reassigned anticodon is a mutant cysteine tRNA and the aminoacyl-tRNA is synthesized in vitro using a cysteinyl tRNA synthase and the mutant cysteine tRNA cannot be recharged by an endogenous cysteinyl tRNA synthetase.

34. The method of claim 29 (i), (ii) or (iii), wherein the reassigned anticodon is an anticodon that is four-fold or six-fold degenerate; optionally wherein the reassigned anticodon is an anticodon for Ile, Ala, Gly, Pro, Thr, Val, Arg, Leu or Ser.

35. The method of claim 29 (i), (ii) or (iii), wherein:
(a) the complement of tRNAs comprises two or more different anticodons that normally encode respective natural amino acids, re-assigned to respective non-natural moieties;
(b) the complement of tRNAs comprises at least one tRNA that comprises an anticodon that is not for a natural amino acid, wherein said anticodon has been re-assigned to a non-natural moiety; optionally wherein said anticodon that has been reassigned to said non-natural moiety is an amber suppressor anticodon.

36. A composition of matter which is:
(i) a composition comprising a complement of tRNAs suitable for translation of a protein comprising at least one non-natural moiety, said complement comprising at least one tRNA that comprises an anticodon for a natural amino acid reassigned to a non-natural moiety, wherein the complement of tRNAs is operable to facilitate translation of an RNA which comprises a codon corresponding to the anticodon that has been reassigned to the non-natural moiety whereby the translated protein may comprise any or all of the twenty natural amino acids; or
(ii) a translation system suitable for translation of a protein comprising at least one non-natural moiety, said system comprising: a complement of tRNAs comprising at least one tRNA that comprises an anticodon for a natural amino acid that has been reassigned to a non-natural moiety; and a translatable mRNA which comprises a codon corresponding to the anticodon that has been reassigned to a non-natural moiety, wherein the mRNA may be transcribed to produce a translated protein that may comprise any or all of the twenty natural amino acids.

37. The composition of matter of claim 36 (i) or (ii), wherein the reassigned anticodon is one of a plurality of different anticodons that is normally for a natural amino acid.

38. The composition of matter of claim 36 (i) or (ii), wherein the aminoacyl-tRNA is synthesized in vitro; optionally wherein:
(a) the tRNA is produced using an RNA synthetase; or
(b) the tRNA is produced using an RNA synthetase and the tRNA is aminoacylated using an RNA synthetase.

39. The composition of matter of claim 36 (i) or (ii), wherein the aminoacyl-tRNA is synthesized in vitro and the tRNA is produced using an RNA synthetase and the tRNA is aminoacylated using an RNA synthetase and the RNA synthetase is selected from PylRS or variants thereof suitable for pyrrolysine tRNA synthase-mediated aminoacylation; Methanococcus jannaschii tyrosyl-transfer RNA synthetase (Mj TyrRS) or variants thereof; Flexizyme; and a cysteinyl tRNA synthetase; optionally wherein:
(a) the tRNA comprising the reassigned anticodon is a mutant cysteine tRNA;
(b) the tRNA comprising the reassigned anticodon is a mutant cysteine tRNA and the aminoacyl-tRNA is synthesized in vitro using a cysteinyl tRNA synthase; or
(c) the tRNA comprising the reassigned anticodon is a mutant cysteine tRNA and the aminoacyl-tRNA is synthesized in vitro using a cysteinyl tRNA synthase and the mutant cysteine tRNA cannot be recharged by an endogenous cysteinyl tRNA synthetase.

40. The composition of matter of claim 36 (i) or (ii), wherein the reassigned anticodon is an anticodon that is four-fold or six-fold degenerate; optionally wherein the reassigned anticodon is an anticodon for Ile, Ala, Gly, Pro, Thr, Val, Arg, Leu or Ser;

41. The composition of matter of claim 36 (i) or (ii), wherein:
(a) the complement of tRNAs comprises two or more different anticodons that normally encode respective natural amino acids, re-assigned to respective non-natural moieties; or
(b) the complement of tRNAs comprises at least one tRNA that comprises an anticodon that is not for a natural amino acid, wherein said anticodon has been re-assigned to a non-natural moiety; optionally wherein said anticodon that has been reassigned to said non-natural moiety is an amber suppressor anticodon.

42. A composition of matter comprising:
(i) a recombinant protein produced by the method of claim 29 (iii);
(ii) a protein library comprising a plurality of recombinant proteins according to (i);
(iii) an mRNA that encodes the recombinant protein of (i) or at least one of the plurality of recombinant proteins in the protein library according to (ii).

43. The composition of matter of claim 42 (i):
(a) which is a macrocyclic protein; or
(b) which comprises one or a plurality of same or different non-natural moieties that facilitate PEGylation, conjugation of small molecules, labelling, immobilisation, intermolecular and/or intramolecular cross-linking or other interactions, formation of higher order structures and/or one or more catalytic activities; optionally which is a macrocyclic protein; or
(c) which comprises two or more of the same or different non-natural moieties that are capable of intramolecular covalent bonding; optionally which is a macrocyclic protein.
